(19) United States
(12) Patent Application Publication
(10) Pub. No.: US 2023/0212533 A1
(43) Pub. Date: Jul. 6, 2023

(54) PRODUCTS AND METHODS FOR THE TREATMENT OF NICOTINE DEPENDENCE

(71) Applicants: THE REGENTS OF THE UNIVERSITY OF MICHIGAN, Ann Arbor, MI (US); The Board of Trustees of Western Michigan University, Kalamazoo, MI (US)

(72) Inventors: James C. Bardwell, Ann Arbor, MI (US); Mark Dulchavsky, Ann Arbor, MI (US); Frederick Stull, Ann Arbor, MI (US)

(21) Appl. No.: 17/928,211

(22) PCT Filed: May 28, 2021
(86) PCT No.: PCT/US21/34824

§ 371 Date: Nov. 28, 2022

Related U.S. Application Data
(60) Provisional application No. 63/032,403, filed on May 29, 2020.

(51) Int. Cl. C12N 9/02; C12N 15/52; C12N 15/78; A61K 38/41; C12N 15/10

(52) U.S. Cl. CPC C12N 9/0093; C12N 15/52; C12N 15/78; A61K 38/415; C12N 15/1065; C12Y 117/03003; C12N 2500/30

(57) ABSTRACT

The disclosure provides variants of nicotine oxidoreductase and methods to select such variants that are unexpectedly active in the catabolic destruction of nicotine by oxidation using oxygen as electron acceptor, and catabolically active fragments thereof. Also disclosed are compositions comprising the CycN cytochrome c protein and at least one of the variant nicotine oxidoreductase holoenzymes, the fragments thereof, or a naturally occurring nicotine oxidoreductase, as well as fusion proteins comprising catalytically active nicotine oxidoreductase fragments or holoenzymes and CycN cytochrome c fragments or holoenzymes. Additionally, variants of L-6-hydroxynicotine oxidase, or catalytically active fragments thereof, are provided. Further disclosed are polynucleotides encoding such proteins, vectors comprising such polynucleotides, and host cells comprising such polynucleotides or vectors. Also provided are methods of using any of the disclosed compositions or formulations to treat nicotine dependence or reduce the risk of relapse to nicotine dependence.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 55531A_Seqlisting.txt; Size: 551,717 Bytes; Created: May 28, 2020.

### FIELD

The disclosure relates generally to the fields of medicine and molecular physiology and more particularly to materials and methods related to addictive dependencies.

### BACKGROUND

Progress in the war on cancer is frustratingly slow. Tobacco consumption is the most important of the preventable causes of cancer. Moreover, smoking not only causes cancer but a host of other cardiovascular and pulmonary problems as well. This makes tobacco consumption the leading cause of disease and preventable death worldwide.³⁵ Smoking in the U.S. is currently responsible for one in five deaths (about 480,000) per year.³² Smokers appreciate this risk, and about 70% of them wish to quit.³ In reality, only a small percentage of smokers succeed in quitting.³³ The addictive power of nicotine, the primary substance driving tobacco consumption, is underappreciated, with 67% of people trying one cigarette becoming daily smokers.³⁴

Flavin-dependent enzymes utilize their flavin adenine dinucleotide (FAD) or flavin mononucleotide cofactors to conduct reduction-oxidation chemistry. These enzymes are able to pass electrons between their cofactor and substrate in either one- or two-electron transfer reactions¹. Some flavoenzymes, termed oxidases, rapidly deliver flavin-bound electrons to molecular oxygen (O₂), creating reactive oxygen species such as superoxide or hydrogen peroxide as a byproduct. Other flavoenzymes are more discerning with their clients, preferring to donate electrons to specific protein or small molecule substrates. These are denoted as dehydrogenases and generally do not rapidly react with molecular oxygen. However, some degree of oxygen reactivity is inevitable in most flavin-dependent enzymes in an aerobic environment because of the inherent reactivity of flavin cofactors with O₂ and oxygen's strongly oxidizing redox potential. Flavin-containing amine oxidases (pfam:01593)² are rapidly re-oxidized by O₂ after accepting electrons from their amine-containing substrates. This rapid re-oxidation is evident, at least in vitro, for nearly all previously characterized members of this enzyme family³⁻⁵. Nicotine oxidoreductase (NicA2), however, appears to defy this conventional wisdom.

Recently, an enzymatic approach to the treatment of nicotine dependence was tried, but did not exhibit the pharmacokinetic and pharmacodynamic properties of a treatment that would be useful in treating human nicotine addiction in the real world. Two recent, independent preclinical studies revealed that intraperitoneal injection of the NicA2 enzyme from Pseudomonas putida S16, an organism that utilizes nicotine as its primary carbon and nitrogen source³⁶, into nicotine-dependent rats largely eliminated nicotine from the blood, reversed symptoms of nicotine withdrawal and extinguished compulsive nicotine consumption^{11,13}. NicA2 treatment also reduced susceptibility to relapse after abstinence in this animal model. Since the avoidance of withdrawal symptoms is one of the key factors sustaining nicotine addiction^{14,35}, these findings are of significant clinical interest.

NicA2 is a FAD-dependent enzyme with the exceptional ability to catalyze the oxidation of nicotine into N-methylmyosmine⁶. It was isolated from Pseudomonas putida S16, a microorganism that has the very unusual ability to grow rapidly using nicotine as its sole carbon and nitrogen source⁷. NicA2 catalyzes the first step in this catabolic pathway, which eventually results in the production of fumarate for the organism's central metabolise. NicA2's FAD cofactor accepts a hydride from nicotine, converting it into N-methylmyosmine in the enzyme's biologically important half reaction⁹. N-methylmyosmine then undergoes spontaneous hydrolysis to pseudooxynicotine, which is both non-toxic and non-addictive in animal models¹⁰.

A major hurdle in the effective use of NicA2 in tobacco cessation therapy of humans, however, is the very slow enzymatic activity of the NicA2 enzyme under the conditions used. In vitro studies showed that the conversion of nicotine to pseudooxynicotine using oxygen as an electron acceptor occurred with an extremely slow k_{cat} of 0.007/sec.³⁷ This is slow, not just for this category of flavin-dependent oxidases, but for enzymes in general. Homologues of NicA2 that catalyze related reactions function with up to 10,000-fold higher turnover rates.³⁸ The poor catalytic rate for NicA2 necessitates the injection of 10 mg/kg of the enzyme into rats on a daily basis to reverse their nicotine dependence, a 330-fold excess over the enzyme's substrate.³⁵ The equivalent dose for a 70 kg human would be an impractical 0.7 gram of enzyme per day.

Thus, a need continues to exist in the art for therapies to treat nicotine dependence, such as enzymatic therapies, but nicotine degrading enzymes such as nicotine oxidoreductase exhibit impractically slow kinetics in nicotine degradation, emphasizing the need for enzymatic therapies to treat nicotine dependence that are sufficiently active in degrading nicotine to provide therapeutic benefit.

### SUMMARY

The disclosure provides products and methods that open up enzyme-based approaches to the treatment of nicotine dependence. The surprising discovery that nicotine oxidoreductase, a central flavin-containing enzyme that degrades nicotine, does not use O₂ as electron acceptor ran counter to the conventional wisdom that flavin-containing enzymes used O₂ as electron acceptor. The kinetics of the oxidative degradation of nicotine by pathways including nicotine oxidoreductase using O₂ as electron acceptor were so slow that the field had turned in other directions for approaches to treat nicotine dependence. The discovery disclosed herein is that the kinetics of redox reactions catalyzed by nicotine oxidoreductase are dramatically improved by coupling the enzyme to an unexpected electron donor in the form of a cytochrome c protein termed CycN cytochrome c. Even more surprising are the directed evolution efforts to modify nicotine oxidoreductase to use O₂, an inexpensive and ubiquitous electron acceptor, instead of a cytochrome c protein with kinetics compatible with a method to treat nicotine dependence. Consistent with this approach, disclosed herein are approximately 100 NicA2 nicotine oxidoreductase variants exhibiting markedly improved nicotine catalysis kinetics relative to the wild-type NicA2 enzyme of SEQ ID NO:131. The substantial number of NicA2 nicotine oxidoreductase variants able to use O₂ as electron acceptor at physiological levels span the full length of NicA2 nicotine oxidoreductase and, collectively, these variants fully characterize the group, or genus, of NicA2 nicotine oxidoreductase variants using O₂ as electron acceptor. These variants, and pharmacologically active fragments thereof as well as polynucleotides encoding such variants and fragments, are useful as therapeutics in the treatment of nicotine dependence, as disclosed in greater detail below. The NicA2 nicotine oxidoreductase variant fragments include fragments of NicA2 nicotine oxidoreductase lacking the approximately 37-residue signal sequence encoded by the full-length gene as well as fragments with truncated N-termini.

Expanding on this enzyme-based approach to treat nicotine dependence, the disclosure also reveals that flavin-containing enzymes analogous to nicotine oxidoreductase, such as L-6-hydroxynicotine oxidase (LHNO), could be modified not to shift the electron acceptor, but to shift the electron donor, i.e., the compound being oxidatively degraded. Native LHNO catalyzes the oxidative degradation of L-6-hydroxynicotine to L-6-hydroxypseudonicotine using O₂ as electron acceptor. Generating LHNO variants to shift the substrate requirement from L-6-hydroxynicotine to nicotine while retaining the capacity to use O₂ as electron acceptor can also be used with kinetics compatible with a method to treat nicotine dependence.

The disclosure also provides screening methods, including high-throughput screening methods, to identify nicotine oxidoreductase variants and L-6-hydroxynicotine oxidase variants useful in degrading nicotine with O₂ as electron acceptor, and methods of using these products to treat nicotine dependence using an enzyme-based approach, which provides a significant expansion of therapies and even therapeutic approaches to treat the major health scourge of nicotine dependence. In one aspect, the disclosure provides a NicA2 nicotine oxidoreductase variant or functional fragment thereof comprising (consisting essentially of or consisting of) fewer than 10 amino acid substitutions, additions, or deletions from the amino acid sequence set forth in SEQ ID NO:131, wherein the NicA2 nicotine oxidoreductase variant exhibits a higher K_{M} and/or a higher K_{cat} for oxidizing nicotine with oxygen as electron acceptor compared to the wild-type NicA2 nicotine oxidase of SEQ ID NO:131. In some embodiments, the NicA2 nicotine oxidoreductase variant or a functional fragment thereof comprises (consists essentially of or consists of) one amino acid substitution from the amino acid sequence set forth in SEQ ID NO:131. In some embodiments, the variant comprises (consists essentially of or consists of) an amino acid sequence that varies from the wild-type sequence of SEQ ID NO:131 at one or more of positions 12, 29, 37, 39, 42, 44, 45, 46, 48, 49, 50, 51, 52, 54, 59, 62, 63, 69, 72, 73, 75, 78, 85, 92, 93, 94, 96, 98, 99, 100, 103, 104, 107, 108, 112, 114, 115, 120, 127, 129, 130, 131, 132, 133, 135, 137, 138, 145, 146, 147, 151, 152, 156, 157, 159, 160, 161, 168, 171, 172, 173, 174, 177, 180, 183, 184, 187, 188, 191, 192, 196, 198, 199, 202, 209, 210, 213, 215, 217, 221, 222, 223, 224, 225, 229, 231, 235, 239, 242, 243, 244, 245, 246, 249, 253, 258, 260, 265, 267, 270, 276, 277, 278, 280, 281, 282, 287, 291, 292, 293, 295, 296, 298, 302, 303, 306, 307, 308, 311, 314, 317, 319, 324, 330, 331, 333, 335, 337, 338, 345, 349, 351, 355, 357, 359, 364, 366, 368, 371, 373, 374, 379, 380, 381, 382, 388, 389, 390, 393, 394, 395, 398, 403, 406, 408, 411, 418, 421, 424, 426, 427, 429, 431, 432, 435, 437, 441, 442, 443, 444, 449, 454, 455, 457, 460, 461, 462, 473, 474, 476, 480, 481, 482, 483, and/or 484.

In some embodiments, the NicA2 nicotine oxidoreductase variant comprises (consists essentially of or consists of) a mature NicA2 nicotine oxidoreductase comprising (consisting essentially of or consisting of) an amino acid sequence that varies from the wild-type sequence of SEQ ID NO:131 at one or more of positions 42, 44, 45, 46, 48, 49, 50, 51, 52, 54, 59, 62, 63, 69, 72, 73, 75, 78, 85, 92, 93, 94, 96, 98, 99, 100, 103, 104, 107, 108, 112, 114, 115, 120, 127, 129, 130, 131, 132, 133, 135, 137, 138, 145, 146, 147, 151, 152, 156, 157, 159, 160, 161, 168, 171, 172, 173, 174, 177, 180, 183, 184, 187, 188, 191, 192, 196, 198, 199, 202, 209, 210, 213, 215, 217, 221, 222, 223, 224, 225, 229, 231, 235, 239, 242, 243, 244, 245, 246, 249, 253, 258, 260, 265, 267, 270, 276, 277, 278, 280, 281, 282, 287, 291, 292, 293, 295, 296, 298, 302, 303, 306, 307, 308, 311, 314, 317, 319, 324, 330, 331, 333, 335, 337, 338, 345, 349, 351, 355, 357, 359, 364, 366, 368, 371, 373, 374, 379, 380, 381, 382, 388, 389, 390, 393, 394, 395, 398, 403, 406, 408, 411, 418, 421, 424, 426, 427, 429, 431, 432, 435, 437, 441, 442, 443, 444, 449, 454, 455, 457, 460, 461, 462, 473, 474, 476, 480, 481, 482, 483, and/or 484. In some embodiments, the variant comprises (consists essentially of or consists of) an amino acid sequence that varies from the wild-type sequence of SEQ ID NO:131 at one or more of positions 93, 104, 107, 108, 130, 132, 249, 317, 368, 379, 381, 427 or 462.

In some embodiments, the variant comprises (consists essentially of or consists of) an amino acid substitution of serine for isoleucine at position 12 (S12I), G29S, S37N, T39S, T42A, R44H, A45I, A45V, S46R, V48A, K49N, G50A, G50C, G50D, G50S, G51S, F52L, Y54F, V59I, G62S, F63L, F63V, A69V, C72S, G73S, Q75H, R78G, R78H, R85C, R85H, T92A, T92I, T92S, F93L, T94A, R96H, R96S, A98E, A98S, G99D, Q100H, E103D, F104I, F104L, A107P, A107T, W108R, L112M, L112Q, P114Q, H115Y, M120I, V127M, E129Q, E129V, D130A, D130E, D130G, D130N, D130S, D130V, D130Y, P131Q, P131S, L132R, T133I, L135M, K137R, T138I, G145A, S146G, S146I, V147F, V147L, S151I, P152S, G156C, G156D, G156S, K157M, K157R, I159V, R160H, I161F, I161V, H168Q, W171C, W171R, E172G, V173A, F174C, F174I, F174V, P177L, P180L, T183I, E184G, E184K, R187L, E188D, K191M, K191N, S192C, S192I, S192N, D196H, I198F, K199R, G202D, A209I, Q210H, S213T, M215L, L217P, E221D, T222S, T223N, D224E, K225N, K225Q, P229L, V231I, F235I, F235L, G239A, Y242N, D243N, A244V, F245Y, M246T, E249G, R253S, T258A, G260A, G260S, M265I, T267I, T267S, G270C, S276C, S276N, V277I, P278Q, P278S, T280K, A281T, V282I, G287D, I291V, K292N, K292Q, K292R, T293S, D295N, D295V, D296E, I298F, I298V, G302A, V303A, M306V, T307P, V308L, N311S, K314Q, G317D, G317S, T319I, K324E, I330T, K331N, G333S, L335V, K337M, G338S, V345L, L349M, R351H, F355L, D357E, Q359L, Q359E, Q359R, W364C, Q366K, Q366R, H368L, H368P, H368R, H368Y, S371R, E373K, L374M, S379N, I380V, T381I, T381S, I382V, I388F, D389E, V390I, R393C, D394N, A395V, R398L, M403I, G406D, E408D, G411 D, G411S, T418S, P421L, L424Q, A426T, W427L, A429V, G431 D, G431S, V432M, L435Q, R437H, L441M, Q442H, A443V, A444G, L449V, E454D, T455A, N457T, H460L, A461V, N462S, A473V, G474S, E476V, L480P, L481A, L481P, S482E, 483L, and/or 484I.

In some embodiments, the variant comprises (consists essentially of or consists of) the sequence set forth in any one of SEQ ID NOs:20-119. In some embodiments, the fragment of a NicA2 nicotine oxidoreductase variant disclosed herein comprises (consists essentially of or consists of) a variant amino acid at a position corresponding to position 93, 104, 107, 108, 130, 132, 249, 317, 368, 379, 381, 427 or 462 of SEQ ID NO:131. In some embodiments, the K_{M} of the variant is greater than 0.114, such as wherein the K_{M} of the variant is at least 1.5, e.g., the K_{M} of the variant is 1.5-29. In some embodiments, the K_{cat} of the variant is greater than 0.007, such as wherein the K_{cat} of the variant is at least 0.132, e.g., the Kcat of the variant is 0.132-0.314. In some embodiments, the NicA2 nicotine oxidoreductase variant disclosed herein comprises (consists essentially of or consists of) one amino acid deletion from the amino acid sequence set forth in SEQ ID NO:131. In some embodiments, the NicA2 nicotine oxidoreductase variant disclosed herein comprises (consists essentially of or consists of) one amino acid addition from the amino acid sequence set forth in SEQ ID NO:131. In some embodiments, the amino acid sequence of the NicA2 nicotine oxidoreductase variant is at least 90% identical to the amino acid sequence set forth in SEQ ID NO:131.

The disclosure contemplates NicA2 nicotine oxidoreductase variants comprising, consisting essentially of, or consisting of any change of fewer than 10 amino acids relative to the wild-type NicA2 nicotine oxidoreductase of SEQ ID NO:131, including insertions, deletions and/or substitutions. For example, a NicA2 nicotine oxidoreductase variant according to the disclosure may exhibit an N-terminal and/or C-terminal truncation relative to the wild-type NicA2 nicotine oxidoreductase of SEQ ID NO:131. The changes relative to the wild-type amino acid sequence may be any form of change, or may be a conservative change, such as the substitution of one polar amino acid for another polar amino acid or one non-polar amino acid for one non-polar amino acid. In addition, the disclosure comprehends the substitution of a non-natural nucleotide, such as dIMP, for a naturally occurring deoxyribonucleotide or a non-natural ribonucleotide, such as IMP, for a naturally occurring ribonucleotide.

Another aspect of the disclosure is drawn to a composition comprising (consisting essentially of or consisting of) a nicotine oxidoreductase and a cytochrome c protein. In some embodiments, the nicotine oxidoreductase is NicA2 nicotine oxidoreductase. In some embodiments, the cytochrome c protein is a CycN cytochrome c protein. In some embodiments, the NicA2 nicotine oxidoreductase comprises (consists essentially of or consists of) the sequence set forth in SEQ ID NO:131, or is a catalytically active fragment thereof, and in some embodiments, the CycN cytochrome c protein comprises (consists essentially of or consists of) the sequence set forth in SEQ ID NO:19, or is a catalytically active fragment thereof. In some embodiments, the nicotine oxidoreductase, or a catalytically active fragment thereof, and the cytochrome c protein, or catalytically active fragment thereof, are joined in a fusion protein. A catalytically active nicotine oxidoreductase fragment has a structure identical to a portion of the full-length nicotine oxidoreductase and functions to catalyze the oxidation of nicotine, using CycN cytochrome c protein as electron acceptor. A catalytically active CycN cytochrome c protein fragment has a structure identical to a portion of the full-length CycN cytochrome c protein and functions as an electron acceptor in the nicotine oxidoreductase-catalyzed oxidation of nicotine. In some embodiments, the composition is contained in an epidermal patch, a liposome, a micelle, an implant, or a nanoparticle.

A related aspect of the disclosure is drawn to a composition comprising (consisting essentially of or consisting of) a polynucleotide encoding the Pseudomonas putida S16 NicA2 nicotine oxidoreductase (i.e., the native sequence of SEQ ID NO:16, the wild-type coding region of SEQ ID NO:130, or a fragment thereof encoding a catalytically active NicA2 nicotine oxidoreductase fragment, or the codon-optimized sequence of SEQ ID NO:12, or a fragment thereof encoding a catalytically active NicA2 nicotine oxidoreductase fragment) and a polynucleotide encoding the CycN cytochrome c protein (i.e., the native sequence of SEQ ID NO:18, or a fragment thereof encoding a catalytically active CycN cytochrome c protein fragment, or the codon-optimized sequence of SEQ ID NO:15, or a fragment thereof encoding a catalytically active CycN cytochrome c protein fragment. In particular, the disclosure provides a polynucleotide encoding the NicA2 nicotine oxidoreductase variant disclosed herein. In some embodiments, the polynucleotide encodes a NicA2 nicotine oxidoreductase variant comprising (consisting essentially of or consisting of) at least one nucleotide variant relative to the wild-type nicA2 coding region of SEQ ID NO:130, wherein the at least one nucleotide variant corresponds to position 35, 85, 110, 115, 124, 131, 133, 134, 136, 143, 147, 148, 149, 151, 156, 161, 175, 184, 187, 206, 214, 217, 225, 232, 233, 235, 253, 254, 274, 275, 277, 280, 286, 287, 292, 293, 296, 300, 309, 310, 312, 319, 322, 334, 335, 339, 341, 343, 360, 379, 385, 386, 388, 389, 390, 391, 392, 395, 398, 403, 410, 413, 434, 436, 437, 439, 452, 454, 466, 467, 470, 475, 479, 481, 504, 511, 513, 515, 518, 520, 521, 530, 539, 548, 550, 551, 560, 564, 572, 573, 574, 575, 586, 592, 596, 605, 625, 630, 638, 643, 650, 663, 664, 668, 672, 673, 675, 686, 691, 703, 705, 716, 724, 727, 731, 734, 737, 746, 757, 772, 778, 779, 795, 800, 808, 826, 827, 829, 832, 833, 839, 840, 841, 844, 860, 871, 874, 875, 876, 877, 883, 884, 888, 892, 905, 908, 916, 919, 922, 932, 940, 949, 950, 956, 970, 989, 993, 997, 1003, 1010, 1012, 1033, 1045, 1052, 1065, 1071, 1075, 1076, 1092, 1096, 1097, 1102, 1103, 1113, 1117, 1120, 1136, 1138, 1141, 1142, 1144, 1162, 1167, 1168, 1177, 1180, 1184, 1193, 1209, 1217, 1224, 1231, 1232, 1252, 1254, 1262, 1271, 1276, 1280, 1286, 1291, 1292, 1294, 1304, 1310, 1321, 1326, 1328, 1331, 1345, 1362, 1363, 1370, 1379, 1382, 1385, 1386, 1418, 1420, 1427, 1439, 1440, 1441, 1442, 1443, 1444, 1445, 1446, 1447, 1448, 1449, 1450, 1451, and/or 1452 of SEQ ID NO:130.

In some embodiments, the variant nucleotide corresponds to position 310, 312, 319, 388, 389, 950, 1052, 1103, 1136, 1280, 1345, 1385, and/or 1386 of SEQ ID NO:130. In some embodiments, the variant nucleotide corresponds to position 310, 312, 319, 388, 389, 950, 1103, 1345, and/or 1385 of SEQ ID NO:130. In some embodiments, the polynucleotide comprises (consists essentially of or consists of) a plurality of nucleotide variations at positions corresponding to positions 1439, 1440, 1441, 1442, 1444, 1445, 1446, 1447, 1449, 1450, 1451 and/or 1452 of SEQ ID NO:130. In some embodiments, the polynucleotide encodes one of the NicA2 nicotine oxidoreductase variants specifically disclosed above. In some embodiments, the polynucleotide comprises (consists essentially of or consists of) the NicA2 variant coding region sequence of nica2mut1, nicA2mut5, nicA2mut6, nicA2mut7, nicA2mut8, nicA2mut9, nicA2mut10, nicA2mut11, nicA2mut12, nicA2mut17, nicA2mut19, nicA2mut20, nicA2mut21, nicA2mut22, nicA2mut23, nicA2mut25, nicA2mut31, nicA2mut35, nicA2mut36, nicA2mut40, nicA2mut43, nicA2mut45, nicA2mut61, nicA2mut64, nicA2mut65, nicA2mut66, nicA2mut75, nicA2mut95, nicA2mut96, nicA2mutD1, nicA2mutH3, nicA2mutH4, nicA2mut101, nicA2mut105, nicA2mut106, nicA2mut112, nicA2mut113, nicA2mut117, nicA2mut118, nicA2mut119, nicA2mut123, nicA2mut130, nicA2mut136, nicA2mut137, nicA2mut138, nicA2mut144, nicA2mut145, nicA2mut146, nicA2mut147, nicA2mut149, nicA2mut152, nicA2mut155, nicA2mut158, nicA2mut160, nicA2mut163, nicA2mut167, nicA2mut169, nicA2mut173, nicA2mut174, nicA2mut175, nicA2mut177, nicA2mut179, nicA2mut180, nicA2mut183, nicA2mut189, nicA2mut191, nicA2mut192, nicA2mut194, nicA2mut198, nicA2mut201, nicA2mut202, nicA2mut204, nicA2mut208, nicA2mut210, nicA2mut214, nicA2mut216, nicA2mut217, nicA2mut219, nicA2mut220, nicA2mut223, nicA2mut228, nicA2mut229, nicA2mut232, nicA2mut233, nicA2mut234, nicA2mut237, nicA2mut239, nicA2mut240, nicA2mut2B5, nicA2mut2D5, nicA2mut2D9, nicA2mut2E3, nicA2mut2E4, nicA2mut2F2, nicA2mut2H3, nicA2mut244, nicA2mut245, nicA2mut249, nicA2mut253, nicA2mut254, nicA2mut255, nicA2mut260, nicA2mut302, nicA2mut303, nicA2mut304, nicA2mut305, nicA2mut306, nicA2mut307, nicA2mut313, nicA2mut314, nicA2mut315, nicA2mut320, nicA2mut321, nicA2mut323, nicA2mut324, nicA2mut325, nicA2mut326, or nicA2mut329.

Polynucleotides comprising, consisting essentially of or consisting of a coding region for a NicA2 nicotine oxidoreductase variant may contain any change of fewer than 10 nucleotides relative to the wild-type nicA2 nicotine oxidoreductase coding region of SEQ ID NO:130, including insertions, deletions and/or substitutions. For example, a polynucleotide according to the disclosure may exhibit a 5′-terminal deletion and/or a 3′-terminal deletion relative to the wild-type coding region sequence set forth in SEQ ID NO:130. The changes relative to the wild-type polynucleotide sequence may include silent mutations due to the degeneracy of the genetic code that do not alter the encoded amino acid sequence, along with at least one change resulting in a NicA2 nicotine oxidoreductase variant being encoded. Polynucleotides according to the disclosure may also contain variant nucleotides relative to SEQ ID NO:130 resulting in codon optimization for improved expression in a particular target organism, such as a human subject receiving treatment for nicotine dependence or at risk of becoming dependent on nicotine.

Additional aspects of the disclosure include, a vector comprising a polynucleotide disclosed herein, and a host cell comprising a polynucleotide or vector disclosed herein. Any vector known in the art may be used, such as any plasmid or any virus suitably engineered for use in delivering a polynucleotide, such as a lentivirus vector system or an adeno-associated virus. Any eukaryotic or prokaryotic host cell useful in amplifying and maintaining polynucleotides and/or nucleic acid vectors is contemplated by the disclosure in addition to the target cells of subjects receiving treatment functioning as host cells.

Yet another related aspect of the disclosure provides embodiments of the above-described composition formulated as a pharmaceutical composition further comprising (consisting essentially of or consisting of) an excipient. In some embodiments, the nicotine oxidoreductase, or catalytically active fragment thereof, is NicA2 nicotine oxidoreductase, or catalytically active fragment thereof. In some embodiments, the cytochrome c protein, or catalytically active fragment thereof, is a CycN cytochrome c protein, or catalytically active fragment thereof. In some embodiments, the composition is contained in an epidermal patch, a liposome, a micelle, an implant, or a nanoparticle. In particular, the disclosure comprehends a pharmaceutical formulation comprising (consisting essentially of or consisting of) the NicA2 nicotine oxidoreductase variant disclosed herein and a pharmaceutically acceptable excipient. In some embodiments, the pharmaceutical formulation comprising (consisting essentially of or consisting of) a NicA2 nicotine oxidoreductase variant further comprises (consists essentially of or consists of) a CycN cytochrome c protein. The disclosure also comprehends a pharmaceutical formulation comprising (consisting essentially of or consisting of) polynucleotide disclosed herein and a pharmaceutically acceptable excipient. In some embodiments of each of the pharmaceutical formulations comprising (consisting essentially of or consisting of) one or more proteins or pharmaceutical formulations comprising (consisting essentially of or consisting of) one or more polynucleotides, the pharmaceutical formulation is contained in an epidermal patch, a liposome, a micelle, an implant, or a nanoparticle.

Another aspect of the disclosure is a L-6-hydroxynicotine oxidase variant comprising (consisting essentially of or consisting of) fewer than 10 amino acid substitutions, additions, or deletions from the amino acid sequence set forth in SEQ ID NO:13. In some embodiments, the L-6-hydroxynicotine oxidase variant comprises (consists essentially of or consists of) one amino acid substitution from the amino acid sequence set forth in SEQ ID NO:13. In some embodiments, the L-6-hydroxynicotine oxidase variant comprises (consists essentially of or consists of) one amino acid deletion from the amino acid sequence set forth in SEQ ID NO:13. In some embodiments, the L-6-hydroxynicotine oxidase variant comprises (consists essentially of or consists of) one amino acid addition from the amino acid sequence set forth in SEQ ID NO:13. In some embodiments, the L-6-hydroxynicotine oxidase variant comprises (consists essentially of or consists of) at least one addition, deletion or substitution for the Asn166 of SEQ ID NO:13, the Tyr311 of SEQ ID NO:13, and the Phe 326 of SEQ ID NO:13. In some embodiments, the addition, substitution or deletion is at least one substitution of a non-polar amino acid for the Asn166 of SEQ ID NO:13, the Tyr311 of SEQ ID NO:13, and the Phe 326 of SEQ ID NO:13. In some embodiments, the amino acid sequence of the L-6-hydroxynicotine oxidase variant is at least 90%, 95%, 97%, 98%, 99%, or 99.5% identical to the amino acid sequence set forth in SEQ ID NO:13. In some embodiments, the L-6-hydroxynicotine oxidase variant further comprises (consists essentially of or consists of) an excipient wherein the nicotine oxidoreductase variant and the excipient are formulated in a pharmaceutical composition. In some embodiments, the pharmaceutical composition is contained in an epidermal patch, a liposome, a micelle, an implant, or a nanoparticle.

Further aspects of the disclosure include a polynucleotide encoding a L-6-hydroxynicotine oxidase disclosed herein, a vector comprising such a polynucleotide, and a host cell comprising such a polynucleotide or vector. In some embodiments, the polynucleotide comprises (consists essentially of or consists of) the sequence set forth in SEQ ID NO:17, which encodes Pseudomonas putida S16 L-6-hydroxynicotine oxidase, or a fragment thereof encoding a catalytically active L-6-hydroxynicotine oxidase fragment. In some embodiments, the polynucleotide comprises (consists essentially of or consists of) the sequence set forth in SEQ ID NO:14, which comprises (consists essentially of or consists of) a codon-optimized coding region for NicA2 nicotine oxidoreductase, or a fragment encoding a catalytically active fragment of NicA2 nicotine oxidoreductase. Embodiments of the vector and host cells provided by the disclosure comprise any of these polynucleotides.

Another aspect of the disclosure is a method of identifying a nicotine oxidoreductase (NicA2) variant using O₂ as an electron acceptor comprising (consisting essentially of or consisting of): (a) culturing a host cell comprising a mutagenized coding region for NicA2 on medium comprising at least 1 mg/mL nicotine; and (b) identifying the mutagenized coding region for NicA2 in a host cell able to grow on the medium as encoding a nicotine oxidoreductase variant using O₂ as an electron acceptor. In some embodiments, the coding region for NicA2 is mutagenized prior to introduction into the host cell. In some embodiments, the coding region for NicA2 is nicA2. In some embodiments, the mutagenized coding region for NicA2 is subjected to at least one more iteration of the above-described method of identifying a nicotine oxidoreductase (NicA2) variant using O₂ as an electron acceptor. In some embodiments, the host cell is Escherichia coli or Pseudomonas putida S16, such as wherein the Pseudomonas putida S16 host cell comprises the partial genotype of ΔnicA2 ΔcycN or ΔcycN alone. In some embodiments, the host cell comprises a coding region for either an iNicSnFR3a nicotine biosensor or an iNicSnFR3b nicotine biosensor, further wherein the host cell culture is subjected to fluorescence-activated cell sorting, further wherein the host cell comprising the coding region for the NicA2 variant using O₂ as electron acceptor is identified if the fluorescence level is lower than a control. See reference 58, incorporated herein by reference in relevant part. In some embodiments, the control is a host cell comprising a coding region for wild-type NicA2 and a coding region for either an iNicSnFR3a nicotine biosensor or an iNicSnFR3b nicotine biosensor.

Yet another aspect of the disclosure is a method of identifying a L-6-hydroxynicotine oxidase (LHNO) variant using nicotine as an electron donor comprising (consisting essentially of or consisting of): (a) culturing a host cell comprising a mutagenized coding region for LHNO on medium comprising nicotine; and (b) identifying the mutagenized coding region for LHNO in a host cell able to grow on the medium as encoding a L-6-hydroxynicotine oxidase variant using nicotine as an electron donor. In some embodiments, the coding region for LHNO is mutagenized prior to introduction into the host cell. In some embodiments, the host cell is Escherichia coli. In some embodiments, the mutagenized coding region for LHNO is subjected to at least one more iteration of the above method drawn to identifying a L-6-hydroxynicotine oxidase (LHNO) variant using nicotine as an electron donor.

Still another aspect of the disclosure provides a method of reducing nicotine dependence in a subject comprising (consisting essentially of or consisting of) administering a therapeutically effective amount of (a) a composition comprising (consisting essentially of or consisting of) a NicA2 nicotine oxidoreductase variant comprising (consisting essentially of or consisting of) fewer than 10 amino acid substitutions, additions, or deletions from the amino acid sequence set forth in SEQ ID NO:131 and an excipient; (b) a composition comprising (consisting essentially of or consisting of) a nicotine oxidoreductase, a cytochrome c protein, and an excipient; or (c) a composition comprising (consisting essentially of or consisting of) a L-6-hydroxynicotine oxidase variant of the sequence set forth in SEQ ID NO:13, wherein the variant comprises (consists essentially of or consists of) at least one addition, deletion or substitution for the Asn166 of SEQ ID NO:13, the Tyr311 of SEQ ID NO:13, and the Phe 326 of SEQ ID NO:13. In some embodiments, the nicotine oxidoreductase is wild-type NicA2 nicotine oxidoreductase. In some embodiments, the cytochrome c protein is the CycN cytochrome c protein. In some embodiments, the L-6-hydroxynicotine oxidase variant comprises (consists essentially of or consists of) a substitution of at least one non-polar amino acid for the Asn166, Tyr311, and Phe 326 of SEQ ID NO:13. In some embodiments, the subject is a current smoker of a tobacco product, i.e., the subject is a former smoker of a tobacco product at risk of relapse. In some embodiments, the subject has ceased use of a tobacco product but is at risk of relapse. In some embodiments, the composition is contained in an epidermal patch, a liposome, a micelle, an implant, or a nanoparticle.

Another aspect of the disclosure is a high throughput screen for NicA2 nicotine oxidoreductase variants using O₂ as electron acceptor comprising (consisting essentially of or consisting of): (a) contacting a plurality of NicA2 nicotine oxidoreductase mutants with nicotine, 10-acetyl-3,7-dihydroxyphenoxazine, and horseradish peroxidase; (b) measuring the production of H₂O₂ by each variant; and (c) identifying a NicA2 nicotine oxidoreductase variant as using O₂ as electron acceptor if the level of H₂O₂ produced is greater than the H₂O₂ produced by a control. In some embodiments, the control is the wild-type NicA2 nicotine oxidoreductase of SEQ ID NO:131. In some embodiments, the H₂O₂ is measured spectrophotometrically or fluorometrically. In some embodiments, the NicA2 nicotine oxidoreductase variant exhibits a rate constant for oxidizing nicotine using O₂ as electron acceptor that is at least 10-fold greater than the rate constant for this reaction using wild-type NicA2 nicotine oxidoreductase.

Other features and advantages of the present disclosure will become apparent from the following detailed description, including the drawing. It should be understood, however, that the detailed description and the specific examples, while indicating preferred embodiments, are provided for illustration only, because various changes and modifications within the spirit and scope of the invention will become apparent to those skilled in the art from the detailed description.

### BRIEF DESCRIPTION OF THE DRAWING

FIG. 1. NicA2 is slow to reoxidize in ambient conditions. NicA2 under ambient conditions was monitored by UV-VIS spectrophotometry in the region of FAD absorbance. Upon addition of nicotine, rapid and complete reduction of FAD was observed and sustained for several minutes. A—absorbance.

FIG. 2. NicA2 is rapidly reduced by nicotine. Oxidized NicA2 was combined with varying concentrations of nicotine and the change in absorbance at 450 nm was monitored by anaerobic stopped-flow spectrophotometry. Note the logarithmic timescale. The resulting traces required three exponentials to fit properly. Inset: plotting the k_{obs} values of the three phases against the concentration of nicotine shows concentration independence. A₄₅₀—absorbance at 450 nm; t(s)—time in seconds.

FIG. 3. Reoxidation of NicA2 by O₂ is slow. (a) NicA2 was reduced by sodium dithionite titration under anaerobic conditions, then mixed with various concentrations of O₂ and monitored for the change in FAD absorbance by stopped-flow spectrophotometry. Inset: a representative trace showing re-oxidation of NicA2's FAD by 540 μM O₂ at 450 nm. These traces were well fit with a single exponential; (b) the k_{obs} values derived from fitting re-oxidation traces were plotted against the O₂ concentration, demonstrating linear dependence.

FIG. 4. A previously unannotated cytochrome c forms an operon with nicA2. (a) the gene annotations when the genome of P. putida S16 was originally sequenced¹⁶; (b) the updated gene annotations currently available from NCBI show an uncharacterized cytochrome c gene WP_080563818.1 between nicA2 and pnao, which we termed cycN.

FIG. 5. cycN knockout is unable to grow on nicotine. Single colonies of P. putida S16 were streaked onto M9 salts agar supplemented with nicotine and imaged after two days of growth at 30° C. The WT strain showed robust growth, whereas the ΔcycN strain grew poorly. Plasmid-based expression of cycN complemented the knockout.

FIG. 6. NicA2 rapidly degrades nicotine in vivo. (a) Quantitative western blots against NicA2 (green) in P. putida S16 lysates show expression of NicA2 as induced by nicotine in both the WT and ΔcycN strains over time. Purified NicA2 of known concentration was included as a loading control; (b) HPLC traces of nicotine concentration from WT cell culture media monitored by absorbance at 254 nm show decreasing concentration of nicotine over time. Inset: nicotine HPLC signals were integrated for both the WT and ΔcycN strains. The WT strain completely degraded nicotine in the culture by 24 hours, whereas a ΔcycN strain was unable to appreciably degrade nicotine over the same time period.

FIG. 7. CycN is reduced by NicA2. Oxidized CycN under ambient conditions was monitored by UV-VIS spectrophotometry. Upon addition of 30 nM NicA2 and 100 μM nicotine, 3.75 μM CycN showed an increase in absorbance typical for reduced cytochrome c at 410 and 550 nm, indicating that CycN had become reduced. Both NicA2 and nicotine are required to produce this change, as adding either one individually failed to reduce CycN.

FIG. 8. NicA2 is rapidly oxidized by CycN. (a) Oxidized CycN and reduced NicA2 were mixed in a series of anaerobic stopped-flow spectrophotometer experiments. Upon mixing, the absorbance changes were dominated by CycN reduction. Inset: The signal was monitored at 542 nm (an isosbestic point for CycN reduction/oxidation) in order to observe spectral changes only associated with NicA2 FAD absorbance. Note the logarithmic timescale. Traces at this wavelength fit well to two exponentials; (b) k_{obs} values determined for the first phase were plotted against the concentration of CycN in the experiment, demonstrating linear dependence. The second phase k_{obs} values were independent of concentration.

FIG. 9. Reduction of NicA2 by dithionite reveals stable semiquinone. Partial reduction of oxidized NicA2 with sodium dithionite produced a species with an increased absorbance from 525-650 nm. The spectrum of the titration point with the highest absorbance in this region is most consistent with a mixed population of oxidized flavin, flavin hydroquinone, and neutral flavin semiquinone. Further titration with sodium dithionite resulted in complete reduction to the hydroquinone (FADH₂) state.

FIG. 10. Reduction of CycN by dithionite. UV-VIS spectra were recorded as sodium dithionite was serially titrated into a solution of oxidized CycN until it was fully reduced. Arrows represent the directionality of change during the titration. Inset: zooming in on just a small section of this titration, an isosbestic point is visible at 542 nm, marked with an arrow. This wavelength was used to monitor the changes in absorbance for NicA2's FAD in the experiments in FIG. 8.

FIG. 11. CycN stopped flow captures the semiquinone state, and the second phase is concentration invariant. (a) Oxidized CycN and reduced NicA2 were combined in an anaerobic stopped-flow spectrophotometer and observed for change in absorbance at 542 nm. When mixed in equimolar amounts, absorbance rose and was maintained at an increased value indicating transition to the flavin semiquinone state. When mixed with excess CycN, NicA2 first reaches the semiquinone state (observable as an increase in absorbance) before becoming fully oxidized (observable as a subsequent decrease in absorbance); (b) k_{obs} values for the second phase of NicA2 re-oxidation by CycN were plotted against the concentration of CycN. k_{obs} is invariant with concentration; (c) signal change for the stopped-flow reaction was also monitored at 552 nm, a wavelength suitable for observing reduction of CycN. The trace required two exponentials with similar amplitudes to fit properly, as one exponential was insufficient. Signal change occurred at the same time as NicA2 oxidation monitored at 542 nm, indicating that the processes occurred simultaneously. Note the logarithmic timescale.

FIG. 12. NicA2 and 6LNO contain a high degree of similarity in the flavin-binding domain. (A) and (B), cartoon renderings of NicA2 (PDB: 6C71) and L hydroxynicotine oxidase (LHNO; PDB: 3K7Q), respectively. The FAD is shown in yellow. (C) and (D) zoomed in view of the residues in close proximity to the isoalloxazine of the FAD (yellow) for NicA2 and LHNO, respectively. Note that the strictly conserved lysine residue (K340 in NicA2 and K287 in 6HLNO) among enzymes of the monoamine oxidase family, which has been shown to be important in accelerating the reaction of FAD with O₂ in some members of the monoamine oxidase family, is present in both enzymes. Nicotine is shown in green and 6-hydroxynicotine is shown in orange.

FIG. 13. NicA2 nicotine oxidoreductase active site. Illustration of NicA2 active site channel blocked by a Lysine residue. Surface rendering of 3NG7 (cyan) aligned with NicA2 (green, PDB 6C71). Residue S329 of 3NG7 corresponds to K385 of NicA2, which when superimposed demonstrates obstruction of the solvent channel.

FIG. 14. P. putida S16 contains terminal oxidases similar to those in other Pseudomonas subspecies. The protein sequence for ccoP2, a cbb₃ cytochrome c oxidase subunit from P. aeruginosa PA01, was used for a NCBI BLAST homology search against the genome of P. putida S16, resulting in two highly significant hits (e values of 7e-168, 4e-163) also annotated as cytochrome c oxidase subunits. These proteins had 67% and 64% sequence identity, respectively. The image alignment was generated using MultAlin software⁵⁷.

FIG. 15. Bovine cytochrome c is not reduced by NicA2. Bovine cytochrome c combined with nicotine and NicA2 did not result in any reduction of cytochrome c over 15 minutes of incubation, unlike the assay performed with CycN (FIG. 7). A—absorbance.

FIG. 16. Nicotine degradation pathway. Schematic illustration of the redox reaction by which nicotine is converted to pseudooxynicotine.

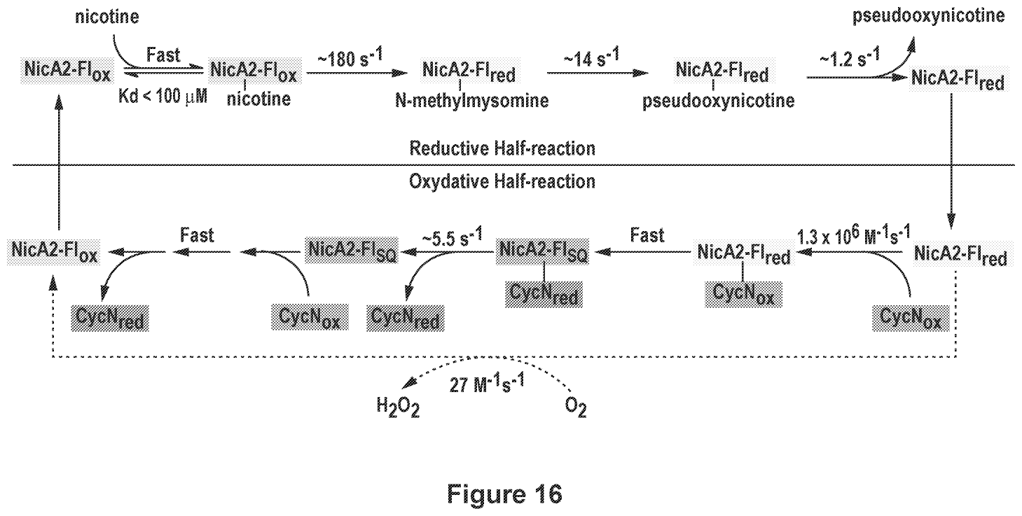

FIG. 17. Substrate binding sites of LHNO and NicA2. (A) and (B), active site views of the residues that interact with L-6-hydroxynicotine in LHNO (PDB 3K7Q) and L-nicotine in NicA2 (PDB 6C71), respectively. FAD is shown in yellow; L-6-hydroxynicotine is shown in green; nicotine is shown in orange; the residues that form hydrogen bonds with the substrates are shown in magenta. (C) and (D), substrate-protein interaction maps for LHNO and NicA2, respectively, made using LigPlot+⁵⁹. Hydrogen bonds are shown in green dashes and hydrophobic interactions are shown as red fans. L-6-hydroxynicotine is likely bound as the lactam tautomer in LHNO based on the hydrogen bonding pattern with Tyr311 and Asn166.

FIG. 18. NicA2 library results in variable growth in a cycN background. The nucleotide sequence of nicA2 codon optimized for E. coli was used as the substrate for an error-prone PCR reaction using the commercially available Genemorph II kit. In this process, nucleotide mutations are dispersed randomly along the coding sequence of nicA2 over the course of a polymerase chain reaction (PCR). The product of this PCR is a mixture of nicA2 derived genes with a variety of different mutations. These mutant libraries were cloned into the vector pJN105 and transformed into P. putida S16 ΔcycN, and then plated onto nicotine containing agar plates to assess for growth. There was a variation in colony size, and large colonies (marked with red arrows) were chosen for sequencing and analysis of activity. All large colonies isolated demonstrated improved oxygen-dependent nicotine-degradation activity of the NicA2 encoded by the mutants.

FIG. 19. Activity of NicA2 mutants. Variant sequences isolated from the selection described herein (e.g., the brief description of FIG. 18) were expressed in E. coli, purified, and then assessed for activity using Michaelis-Menten kinetics. The kinetic parameters determined by the assay are listed here in comparison to the wild-type enzyme of SEQ ID NO:131.

### DETAILED DESCRIPTION

Disclosed herein are products and methods useful in providing enzyme-based therapies to treat nicotine dependence. The disclosure provides methods to obtain nicotine oxidoreductase variants that oxidize nicotine by transferring electrons to O₂ rather than the NicA2 cytochrome c protein and methods to obtain such variants, as well as L-6-hydroxynicotine oxidase variants that transfer electrons to O₂ from nicotine rather than the natural substrate of L-6-hydroxynicotine, and methods to obtain such L-6-hydroxynicotine oxidase variants. Also disclosed are screening methods for such variants, the substantial number of such variants obtained from such screening, and methods of administering the variants to treat nicotine dependence. In addition, the disclosure provides methods of treating nicotine dependence comprising administering a nicotine oxidoreductase variant, such as a NicA2 nicotine oxidoreductase variant, e.g., one of the approximately 110 variants disclosed herein, or a catalytically active fragment thereof, to a patient in need, such as a current smoker or an individual at risk of becoming a smoker of a nicotine-containing product such as tobacco. Analogous treatment methods provide for the administration of a therapeutically effective amount of an L-6-hydroxynicotine oxidase variant, or catalytically active fragment thereof, to such a patient in need. Another treatment method provided by the disclosure is a method of treating nicotine dependence by administering a therapeutically effective amount of a pharmaceutical composition comprising a nicotine oxidoreductase, such as NicA2 nicotine oxidoreductase, and a CycN cytochrome c protein to such a patient in need. In some embodiments, the nicotine oxidoreductase and CycN cytochrome c protein are present in the pharmaceutical composition as a fusion protein.

The disclosure discloses and contemplates nicotine oxidoreductase variants such as NicA2 nicotine oxidoreductase variants, or catalytically active fragments thereof, that are at least 90%, 95%, 97%, 98%, 99%, or 99.5% identical to the corresponding region of the wild-type NicA2 nicotine oxidoreductase (SEQ ID NO:131). In like manner, L-6-hydroxynicotine oxidase variants according to the disclosure are at least 90%, 95%, 97%, 98%, 99%, or 99.5% identical to the corresponding region of the wild-type L-6-hydroxynicotine oxidase (SEQ ID NO:13). Similarly, polynucleotides encoding nicotine oxidoreductase variants, such as NicA2 nicotine oxidoreductase variants, of the disclosure are at least 90%, 95%, 97%, 98%, 99%, or 99.5% identical to the corresponding region of the polynucleotide encoding the wild-type nicotine oxidoreductase, such as polynucleotides of SEQ ID NO:12 or SEQ ID NO:16, and polynucleotides encoding L-6-hydroxynicotine oxidase variants of the disclosure are at least 90%, 95%, 97%, 98%, 99%, or 99.5% identical to the corresponding region of the polynucleotide encoding the wild-type L-6-hydroxynicotine oxidase, such as polynucleotides of SEQ ID NO:14 or SEQ ID NO:17.

The percent identity (i.e., percent homology) between a variant amino acid sequence and the wild-type amino acid sequence is a function of the number of identical positions shared by the sequences, taking into account the number of gaps, and the length of each gap, which need to be introduced for optimal alignment of the two sequences. Analogously, the percent identity of two polynucleotide sequences is a function of the number of identical positions share by the two polynucleotide sequences. For catalytically active fragments of nicotine oxidoreductase variants or L-6-hydroxynicotine oxidase variants, or their encoding polynucleotides, the percent identities reflect a comparison of the sequence of the fragment to the portion of the wild-type molecule that corresponds to the fragment. The comparison of sequences and determination of percent identity between two sequences can be accomplished using a mathematical algorithm, as described below.

The percent identity between two nucleotide sequences can be determined using any algorithm known in the art, such as the GAP program in the GCG software package, using a NWSgapdna. CMP matrix and a gap weight of 40, 50, 60, 70, or 80 and a length weight of 1, 2, 3, 4, 5, or 6. The percent identity between two polynucleotide or amino acid sequences can also be determined using the algorithm of Meyers et al. (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm, which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6.

In developing the aforementioned aspects of the disclosure, we understood that enzymes almost always function in catalytic quantities and we reasoned that the NicA2 nicotine oxidoreductase needed to perform much better than seen in in vitro assays of the enzyme to enable P. putida S16 to grow on nicotine. In fact, we calculated that the actual turnover number for nicotine oxidase in vivo are estimated to be at least 500-fold faster (k_{cat}: 1/sec; see Example 1 for details of the calculation) to enable P. putida S16 to grow on nicotine with a reported doubling time of one hour.³⁹ This realization led to further investigation, which revealed that nicotine oxidoreductase (NicA2) defied the conventional wisdom that amine oxidases are oxidized by molecular oxygen. To explore the possibility that NicA2 could be a significant component of an effective treatment for nicotine dependency and for the reduction of relapse to nicotine dependency, we biochemically characterized NicA2, including discovery of its in vivo electron acceptor. In addition, we are using protein design and directed evolution to enhance the inherent capacity of NicA2 to utilize O₂ as an electron acceptor or using directed evolution to enhance the inherent capacity of 6 hydroxynicotine oxidase to degrade nicotine, followed by testing the improved variants.

NicA2 nicotine oxidoreductase belongs to the class of flavoenzymes that typically cycle their bound flavin between an oxidized and a reduced state, and require an electron acceptor to reoxidize the flavin.⁴⁰ Although some flavoenzymes have been shown to efficiently use oxygen as their electron acceptor, all flavin-dependent enzymes are at least slowly oxidized by molecular oxygen simply because of oxygen's strongly oxidizing redox potential. We explored the possibility that NicA2 in Pseudomonas donates its electrons not to oxygen but to a different electron acceptor. We searched for the in vivo electron acceptor of NicA2 using a biochemical approach, fractionating extracts from P. putida S16 to find proteins or small molecules that are capable of accelerating NicA2's reoxidation. We will also consider the possibility that multiple electron acceptors are involved in NicA2 reoxidation that act either sequentially or alternatively.

Flavins have the advantage of providing easily quantifiable spectroscopic signals indicating their different oxidation states. We can therefore easily monitor the oxidized form of the enzyme as it reacts with nicotine, determine the rate of that reaction, and determine if there is any product inhibition. Alternatively, we can watch the reduced enzyme react with oxygen or other electron acceptors, and determine the rates of these reactions. The simple spectroscopic assays that we developed and disclose herein allow us to monitor not just multiple turnover reoxidation reactions involving NicA2, but single turnover experiments as well. These methods are particularly useful in identifying in vivo electron acceptor(s) because the acceptor(s) should be able to rapidly react with NicA2 and thus be detectable without having to reconstruct the entire pathway.

More particularly, NicA2 receives two electrons from nicotine. In order for subsequent rounds of catalysis to continue, the two electrons retained on NicA2's FAD cofactor from nicotine oxidation must be efficiently transferred to an electron acceptor. Given NicA2's homology to flavin-dependent amine oxidases that transfer their electrons directly to O₂, studies to date have assumed that O₂ directly accepts the electrons from NicA2's reduced FAD^{9,12}. However, the in vitro characterization of NicA2 using O₂ as a terminal electron acceptor for the reaction revealed that NicA2 has a turnover number of 0.007 s^{-1 15}. This turnover number is abysmally low, especially when compared with most flavin-containing amine oxidases (e.g., monoamine oxidase) that have turnover numbers of about 10-100 s^{-1 38}. NicA2's very poor activity as an oxidase in vitro presents a major issue in developing the enzyme into a nicotine-cessation therapy: treatment has required prohibitively high doses of NicA2 to achieve symptomatic relief of nicotine-dependent behavior in rat models—up to 70 mg/kg¹¹—which is likely a consequence of the low catalytic activity of the enzyme.

To help understand the basis for the discrepancy between NicA2's low catalytic activity in vitro and its ability to sustain rapid growth on nicotine, we conducted a search for alternative, more efficient, electron acceptors for NicA2. In this study, we demonstrate both in vivo and in vitro that a novel cytochrome c protein, named CycN, is responsible for accepting electrons from reduced NicA2 in vivo, not O₂. Our demonstration that a member of the flavin-dependent amine oxidase superfamily uses a redox cofactor other than O₂ as an in vivo electron acceptor opens up new avenues for the use of NicA2 in treating nicotine dependence and leads to the expectation that other members of this enzyme superfamily use alternative physiologic electron acceptors as well.

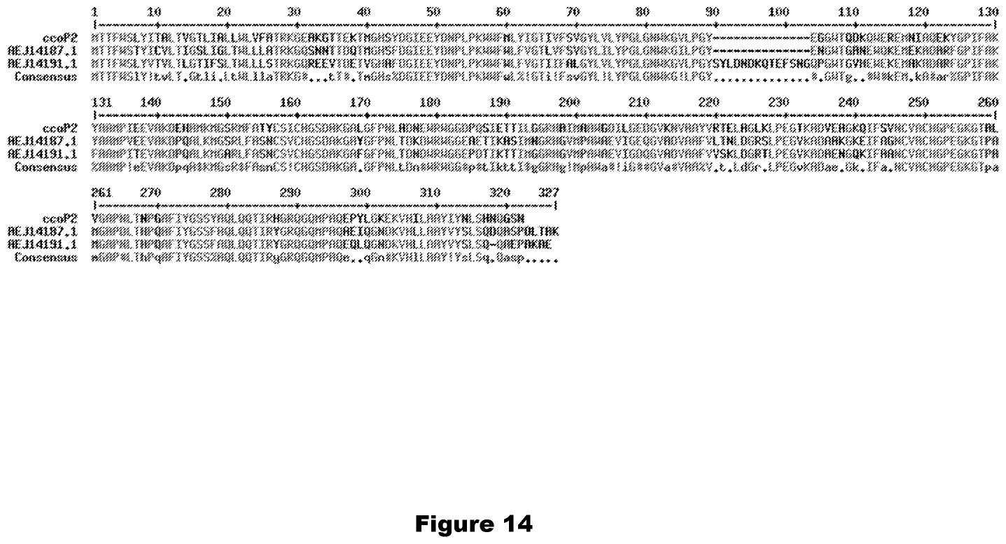

We have demonstrated that a newly identified cytochrome c, CycN, is an effective redox partner for NicA2, whereas O₂ is not. That NicA2 cannot donate its electrons efficiently to oxygen indicates that NicA2 is not an oxidase as was previously thought, but should be categorized as a dehydrogenase. This makes NicA2 a notable outlier within the flavin-containing amine oxidase superfamily. We found that hydride transfer between nicotine and NicA2's FAD in the reductive half reaction occurs rapidly. This is followed by two subsequent kinetic events that may correspond to hydrolysis of N-methylmyosmine and release of the final product, pseudooxynicotine. Re-oxidation of NicA2's FADH₂ by CycN in the oxidative half reaction must occur in a sequence of two electron transfer reactions, given that each molecule of CycN receives only one electron. Based on our data, electron transfer to the first CycN appears to be rate-limited by the interaction between NicA2-Fl_{red} and CycNₒₓ, whereas transfer of the electron to the second CycNₒₓ seems to be limited by dissociation of the first CycN. These points are summarized in the kinetic mechanism shown in FIG. 14. Oxidation of FADH₂ by O₂ is unlikely to contribute significantly to nicotine turnover by NicA2 in vivo because O₂ oxidizes NicA2-Fl_{red} with a bimolecular rate constant about 45,000 times lower than its oxidation by CycN.

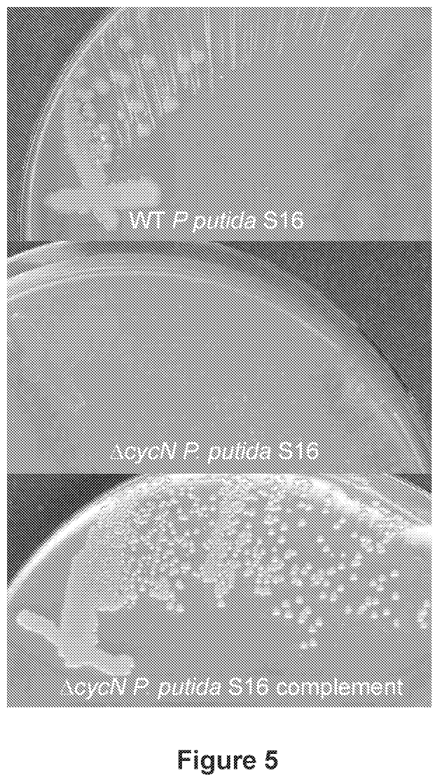

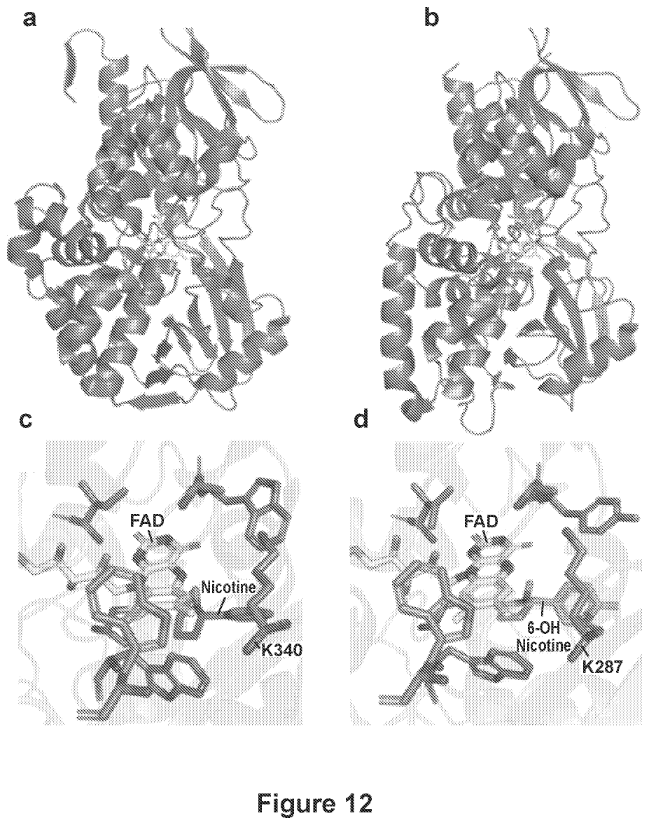

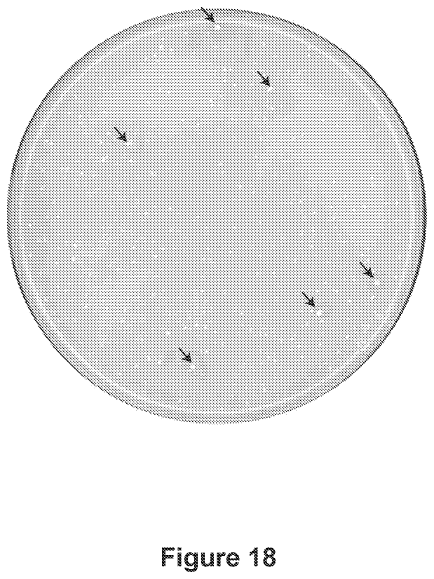

The discovery that NicA2 is a dehydrogenase that uses CycN as an electron acceptor raised a number of interesting questions about the mechanism by which NicA2 discriminates between CycN and O₂. NicA2 appears to be specific for CycN because bovine cytochrome c is a poor recipient of electrons from NicA2 containing reduced flavin, indicating that CycN contains structural features that either optimize the reactivity of its heme cofactor and/or are important for binding to NicA2. The published structures for NicA2, however, show that the isoalloxazine (the redox active portion) of NicA2's FAD cofactor is buried within the core of the protein, at least 10 Å away from the surface of the protein. Can one-electron transfers between the isoalloxazine of NicA2 and the heme of CycN span this distance, or are major conformational changes and/or radical relay networks required for electron transfer between these two redox centers to occur? NicA2's poor reactivity with O₂ is even more difficult to rationalize based on the published structures, as NicA2's FAD binding site is very similar to that in a closely homologous protein that reacts rapidly with O₂, 6-hydroxy-L-nicotine oxidase (6HLNO) (FIG. 12). Curiously, NicA2 even has the strictly conserved lysine near N5 of the isoalloxazine that has been shown to be important for reactivity with O₂ in flavin-containing amine oxidases²². A recent study attempted to identify NicA2 variants with improved O₂-dependent nicotine-degrading activity by screening a library of NicA2 variants at residues near the isoalloxazine of the FAD¹². However, NicA2 variants with only very modest improvements in performance were identified, indicating that O₂ reactivity in NicA2 is not controlled by structural features within the immediate vicinity of the flavin's isoalloxazine. Selections for mutants in NicA2 that are able to bypass the need for CycN are expected to yield, and have yielded, enzymes better suited to use O₂ as an electron acceptor than wild-type NicA2. The strong growth defect of cycN deletion mutations on plates that supply nicotine as the sole carbon and nitrogen source (FIG. 5) provides a very convenient and powerful way to select for such cycN bypass mutants, as shown in FIG. 18.

Microorganisms are astonishingly versatile in their ability to thrive in conditions with only minimal nutrients. In such limiting conditions, they find ways to obtain energy and nutrients from surprising sources. For P. putida S16, CycN provides an illustration of one such adaptation. Rather than transferring electrons derived from nicotine from NicA2 directly to O₂, which would simultaneously waste valuable reducing equivalents and create reactive oxygen species like H₂O₂, electrons are shuttled from NicA2 to CycN instead. However, cytochrome c proteins are not known as terminal electron acceptors. Thus, the electrons obtained by CycN from nicotine oxidation must then be passed to another electron acceptor to enable continued turnover by NicA2. Where these electrons are transferred, and their eventual fate, is unknown. Other Pseudomonas subspecies conduct aerobic respiration through an electron transport chain^{23,24}, and P. putida S16 seems to contain the same machinery (FIG. 14) providing one possible avenue for CycN re-oxidation. Regardless of the exact pathway the electrons take from CycN, it is clear that CycN is required for robust growth of the organism on nicotine.

NicA2 has been investigated as a potential intravenous medication for treating nicotine dependence in rats^{10,11}, but a prohibitively large amount of protein has been required to achieve effective treatment—at least 10 mg/kg daily¹¹, considerably more than is feasible for injection into humans. The average adult weighs 62 kg. This would necessitate daily injections of more than a half a gram of protein, which is an exorbitant amount. For this reason, there is interest in generating a NicA2 variant with increased in vitro turnover rate with nicotine. One avenue for enhancing therapeutic nicotine turnover resulting from our work may be to co-administer CycN alongside NicA2. Another option would be to engineer the enzyme to enhance its ability to use O₂ as an electron acceptor. This may be possible given that NicA2 belongs to an enzyme family where most members react rapidly with O₂, and the data disclosed herein relating to the large number of such variants obtained to date, realizes that possibility. The aforementioned medium-throughput screen of NicA2 variants attempted to do just that¹². Performing site-saturation mutagenesis of the active site of NicA2, Thisted et al. discovered a number of mutations that allowed for an increased turnover rate of nicotine with oxygen as the electron acceptor.¹² Their best variant provided 19 times the activity of wild-type. This is a modest increase given that NicA2 can re-oxidize at more than 45,000 times the rate of re-oxidation with O₂ when provided its physiologic electron acceptor, and indicates that activity with O₂ could be further improved.

The disclosed subject matter will be better understood by referring to the following Examples.

### EXAMPLES

### Example 1

Materials and Methods

Strains and Culture Conditions

Pseudomonas putida S16 was obtained from ATCC. Culture was performed in lysogeny (i.e., Luria Bertani) broth (LB) media unless otherwise specified. M9-nicotine media was made with the following: 6 g/L Na₂HPO₄, 3 g/L KH₂PO₄, 1 mM Mg SO₄, 0.1 mM CaCl₂), 1 μg/mL thiamine, and 1 g/L nicotine. M9-nicotine agar was made with the same recipe, with an additional 15 g/L bacto-agar (Thermo Fisher Scientific). All liquid cultures were inoculated from single colonies struck out onto selective media. Escherichia coli BL21 (DE3) was used for protein expression. Protein expression media (PEM) contains 12 g L⁻¹ tryptone, 24 g L⁻¹ yeast extract, 50.4 g L⁻¹ glycerol, 2.13 g L⁻¹ K₂HPO₄, and 12.54 g L⁻¹ KH₂PO₄.

Construction of Vectors

Standard cloning techniques were used. pEC86 helper vector was obtained from the Culture Collection of Switzerland. pJN105 vector was obtained as a gift from Ute Römling (Karolinska Institute). Genes codon optimized for expression in E. coli were purchased from Genscript for cycN and nicA2 and cloned via restriction digest into pET28a, pJN105, or pET22b vectors. The nicA2 gene, including its N-terminal signal sequence, was cloned into pET28a containing an N-terminal His-SUMO tag. For the complementation experiments, full-length cycN, including its native signal sequence, was cloned into pJN105. For protein expression and purification, the sequence for mature cycN lacking its signal sequence was cloned downstream of the pelB leader sequence in pET22b.

Estimating NicA2 Turnover Rate In Vivo

When grown on nicotine as its sole carbon and nitrogen source, P. putida S16 obtains nearly all of its biomass from this metabolite. Using the known values for doubling time, bacterial mass, and the amount of nicotine that must be turned over to sustain growth, we can estimate the minimal nicotine turnover rate for NicA2. A single bacterium's carbon and nitrogen content has a mass of about 0.3 pg, P. putida S16 doubles in about 90 minutes when grown on nicotine⁷, and nicotine has a molecular weight of 162 g mol⁻¹. Therefore, each bacterium must turn over 2×10⁻¹⁵ mol of nicotine to double its mass. Assuming that NicA2 accounts for no more than 5% of total cell mass, there can be no more than 2.8×10⁻¹⁹ mol of NicA2 expressed for a single cell. We thus estimate the NicA2 in vivo turnover rate as 2×10⁻¹⁵ mol nicotine degraded by 2.8×10⁻¹⁹ mol NicA2 in 90 minutes, resulting in a turnover rate of about 1.3 s⁻¹.

NicA2 Expression and Purification

The pET28a-based expression vector for NicA2 was transformed into E. coli BL21 (DE3) cells and grown in 4 L PEM at 37° C. with shaking to an OD₆₀₀ of 1.0. The temperature was then lowered to 20° C. and expression was induced with 100 μM IPTG. The culture was grown overnight at 20° C. After harvesting, the cells were lysed at 4° C. by sonication in 50 mM Tris HCl, 400 mM NaCl, 15 mM imidazole, 10% glycerol, pH 8.0 (lysis buffer) with DNase I and cOmplete™ protease inhibitor cocktail. The lysate was cleared by centrifugation and the supernatant was loaded on three 5 mL HisTrap columns pre-equilibrated in lysis buffer. The columns were washed with 20 mL lysis buffer, then 20 mL lysis buffer+20 mM imidazole, and NicA2 was then eluted in lysis buffer+0.5 M imidazole. NicA2 was then exchanged into 40 mM Tris-HCl, pH 8+0.25 M NaCl in the presence of Ubiquitin-like-specific protease 1 (ULP1) to cleave off the His-SUMO tag and subsequently passed over the HisTrap column again in 25 mM Tris HCl, pH 8+0.2 M NaCl to remove the His-SUMO tag. Protein was then exchanged into 25 mM Tris pH 8.5 and loaded onto three 5 mL HiTrap Q columns equilibrated with the same buffer. NicA2 was eluted by salt gradient using 25 mM Tris pH 8.5+1 M NaCl. Fractions containing NicA2 were concentrated, then run over a HiLoad Superdex 200 column in 40 mM HEPES 100 mM NaCl pH 7.4. Purified protein was concentrated and flash frozen, then stored at −80° C. until use.

CycN Expression and Purification

The methods in enzymology subsection of the cited Londer reference, which describes the expression and purification of cytochromes c, was followed⁵⁶. Antibiotic selection was maintained with chloramphenicol 17 μg mL⁻¹ and ampicillin 50 μg mL⁻¹ throughout. All incubations were performed at 30° C. E. coli BL21 (DE3) cells were transformed with both pEC86 helper vector and pET22b-cycN for periplasmic expression. A single colony of the resulting transformation was inoculated into overnight culture, then subcultured into 3L PEM and incubated at 200 rpm. Cultures were grown to a density of OD 0.6-0.8, then induced with 10 μM IPTG. Cells were left to express overnight for 18-22 hours. Red pellets were visible after spinning down at 4,000 g for 20 minutes.

Pellets were immediately resuspended for periplasmic extraction by osmotic shock in ice-cold osmotic shock buffer (0.5 M sucrose, 0.2 M Tris HCl, pH 8.0, and 0.5 mM EDTA), 50 mL buffer per L of culture. 33 mL ice-cold water was added after resuspension, and the resulting mixture was incubated on ice with gentle shaking for 2 hours. Suspensions were spun down at 12,000 g for 20 minutes, and the red supernatant saved. These supernatants were dialyzed against 4 L of 30 mM sodium citrate+38 mM Na₂HPO₄ pH 4.0 overnight, then dialyzed into an additional 4 L of 25 mM NaH₂PO₄ at pH 4.0. The next day, samples were loaded onto a HiTrap SP HP cation exchange column (GE Life Sciences) equilibrated in the same buffer, and eluted against 25 mM NaH₂PO₄, pH 4.0+1 M NaCl in a salt gradient. Final cleanup was performed by running over a HiLoad Superdex 75 size exclusion column equilibrated in 40 mM HEPES 100 mM NaCl pH 7.4. Purified protein was flash frozen and stored at −80° C. until use.

Construction of Mutant Libraries

The nucleotide sequence of nicA2 codon optimized for E. coli was used as the substrate for an error-prone PCR reaction using the commercially available Genemorph II kit. In this process, nucleotide mutations are dispersed randomly along the coding sequence of nicA2 over the course of a polymerase chain reaction (PCR). The product of this PCR is a mixture of nicA2 derived genes with a variety of different mutations. These mutant libraries were cloned into the plasmid backbone pJN105 using standard molecular biology techniques for expression in Pseudomonas putida S16. Additionally, mutant libraries were purchased already cloned into pJN105 from GENEWIZ (South Plainfield, N.J.).

Selection of Mutant Libraries

The pJN105-nicA2 mutant libraries constructed above were transformed into Pseudomonas putida S16 ΔcycN via electroporation. The resulting transformants were plated onto M9 agarose plates with nicotine as the sole carbon source, or inoculated into M9 liquid media with nicotine as the sole carbon source, to enrich for nicA2 mutants with increased oxygen-dependent nicotine degrading activity. Clones isolated from this selection were inoculated into LB medium supplemented with gentamycin, and their respective pJN105-nicA2 plasmids were isolated. These plasmids were subjected to Sanger sequencing to determine the nucleotide sequences of nicA2 variants with increased oxygen-dependent activity. The amino acid sequences of the encoded NicA2 protein variants were inferred from the encoding polynucleotide sequences.

Generation of cycN Knockout

A cycN knockout was generated by two-step allelic exchange according to the protocol established by Hmelo et al.¹⁸ Briefly, PCR of P. putida S16 genomic DNA was used to amplify regions upstream and downstream of cycN using the listed primers (Table 1) for “up” and “down” fragments, partially including the start and end of the gene. An additional PCR reaction assembled the fragments together, creating a substrate for homologous recombination against the P. putida S16 genome. This was cloned into pEX18-Gm vector by restriction digest. Upon recombining, the P. putida S16 homologous fragment inserts next to the cycN gene along with the pEX18-Gm sequence containing a gentamycin marker and sacB marker for sucrose counterselection. pEX18-Gm cannot replicate in P. putida S16, and gentamycin resistance can only be passed on in this strain by genomic integration. Therefore, by first selecting for gentamycin resistance, we generate clones with the integrated genomic marker. Then, counterselecting by plating onto sucrose afterward, we select for clones that undergo a secondary recombination event, removing remaining pEX18-Gm sequence with sacB and the majority of cycN coding sequence, resulting in a scarless knockout. pEX18-Gm was electroporated into the mating strain E. coli S17. Both E. coli S17 and P. putida S16 strains were grown at 30° C. until an OD₆₀₀ of 1.0 was reached, at which point 5 mL of culture was spun down and resuspended in 1 mL LB. An equal mixture of each strain, 200 μL each, was then spotted onto an LB agar plate for mating overnight at 30° C. The next day, the spot was scraped and washed three times with 1 mL 150 mM NaCl. The resulting washed cells were resuspended in 500 μL 150 mM NaCl, and serial dilutions were plated on M9 salts+0.4% glucose+25 μg mL⁻¹ gentamycin plates for selection of Pseudomonas with genomic integration of antibiotic marker. This resulted in more than 100 colonies, 8 of which were re-streaked onto 20% sucrose no-salt LB agar for secondary selection. This resulted in many single colonies, 16 of which were chosen for colony PCR screening using the listed verification primers (see Table 1—Primers), identifying which colonies successfully lost cycN by elimination through recombination. Three colonies appeared positive by size of PCR band; these were then gel extracted and submitted for Sanger sequencing, confirming the location and fidelity of knockouts.

TABLE 1
Primers

| | | SEQ |
|---|---|---|
| | | ID |
| Name | Sequence | NO |
| CycN_del_ | TCCTCTAACTGAAACTT | 1 |
| down_F | TTGAAAG | |
| CycN_del_ | ATCCGGGGATCCGCTCT | 2 |
| down_R | TTTGACATTATCCTCTG | |
| CycN_del_ | ATCCGGGAATTCGTTCATG | 3 |
| up_F | TTAAGCAGAATCTCG | |
| CycN_del_ | CTTTCAAAAGTTTCAGTTA | 4 |
| up_R | GAGGACGCTTGTTTCATTT | |
| | TTTATCCTC | |
| CycN_ | GATTCATCAAAGAGGGGCAG | 5 |
| verify_F | | |
| CycN_ | GTATTCGTTACAGGCAGAGC | 6 |
| verify_R | | |

Phenotyping Wild-Type, Knockout, and Complemented P. putida S16

Single colonies of each strain grown from LB agar plates were streaked onto M9 salts+1 mg mL⁻¹ nicotine plates to examine for growth. Only the wild type (WT) grew well on these plates. WT and ΔcycN strains were grown overnight in LB media and electroporated with pJN105 empty vector and pJN105-cycN for the complementation assay. These were plated onto LB+25 μg mL⁻¹ gentamycin selection plates. Single colonies from these plates were then streaked onto fresh plates containing M9 salts+1 mg mL⁻¹ nicotine, additionally supplemented with 25 μg mL⁻¹ gentamycin and 0.01% arabinose to observe for growth at 30° C. for 2-5 days. To increase the stringency of the selection lower levels of arabinose were used. We also were able to perform the selection in liquid culture contained in a bioreactor.

In Vitro Assays

The buffer used in all in vitro experiments was 40 mM HEPES-KOH, pH 7.5, 100 mM NaCl, 10% glycerol. NicA2 is in its fully oxidized form under ambient conditions in the absence of nicotine. The absorbance spectrum of oxidized FAD was used to determine the concentration of NicA2, using an extinction coefficient of 11,300 M⁻¹ cm⁻¹ at 450 nm. 20 μM NicA2 was combined with 40 μM nicotine and rapidly transferred to an absorbance cuvette to measure reduction of NicA2 under ambient conditions.

CycN was fully oxidized by addition of 5 mM ferricyanide. Excess ferricyanide was then exchanged out of the sample by running over a PD-10 desalting column before use. Concentrations of CycN were determined using the extinction coefficient of oxidized cytochrome c at 410 nm (101,600 M⁻¹ cm⁻¹). NicA2 and CycN were observed for characteristic spectrophotometric changes in a Shimadzu UV-1900 UV-VIS spectrophotometer. 3.75 μM CycN was combined with either 100 μM nicotine alone, 30 nM NicA2 alone, or both together and monitored for change between 250-600 nm. The same assay was performed with bovine cytochrome c (Sigma-Aldrich), except that the concentration of bovine cytochrome c was 6.84 μM. The bovine cytochrome c assay was monitored for change in absorbance in the same region (250-600 nm) for 15 minutes. Oxidized CycN was additionally titrated with increasing amounts of sodium dithionite to achieve a fully reduced state, with absorbance scans taken at each titration step.

Nicotine Degradation Assay

This assay was performed to determine both the amount of nicotine degraded and the concentration of NicA2 enzyme in cultures of P. putida S16 grown in the presence of nicotine. Both wild-type and ΔcycN strains were grown overnight in LB. The next day, these cultures were diluted to OD 0.1 in 5 mL M9 salts+0.4% glycerol and allowed to grow for 2 hours. At this time point, nicotine was added to a final concentration of 1 mg mL⁻¹ in each sample. The point of nicotine addiction was considered time=0. From then on, the cultures were sampled at 2, 4, 8, 24, and 49 hours. 200 μL of culture was extracted at each time point and spun down at 16,000 g for 10 minutes. The supernatant was isolated, and 100 μL supernatant was mixed with 300 μL methanol to prepare HPLC samples. The cell pellet was resuspended in 100 μL Bacterial Protein Extraction Reagent (B-PER; Thermo Fisher) and allowed to incubate at room temperature for 15 minutes to complete lysis. After this time had elapsed, 25 μL of 5× reducing gel loading buffer was added to each sample.

Samples for HPLC were further clarified by spinning at 16,000 g for 30 minutes. 100 μL of each clarified sample was placed into autosampler vials. These were injected, then separated, for analysis using a Vydac C18 4.6×250 mm column (Catalog: 218TP54) and an isocratic water+0.1% TFA mobile phase. A nicotine standard concentration gradient from 10 mM down to 1 μM was run. 10 μL of standards and experimental samples were injected for analysis. Samples and standards were within the linear range of detection, and the absorbance peaks were integrated for quantification.

Samples for western blot analysis were boiled for 5 minutes. Protein standards of purified NicA2 were prepared at known concentrations from 1 μM to 1 nM. 10 μL of each sample and standard were loaded onto a Bio-Rad 12% SDS-reducing gel and run at 150 V until completion. The gel was transferred to nitrocellulose blot via the Trans-Blot Turbo system (Bio-Rad). Blots were blocked in 5% milk TBST and stained with 1:10,000 rabbit-derived NicA2 antisera (Pacific Immunology) overnight at 4° C. Blots were washed with 5% milk TBST three times, and then incubated with 1:20,000 goat anti-rabbit IR800 dye (LI-COR Biosciences) for 2 hours at room temperature. Membranes were washed with TBST and then imaged using a LI-COR Odyssey Clx. Protein bands were quantified using LI-COR Odyssey software, and the NicA2 standard curve was used to determine the linear range of detection and concentration of NicA2 at each time point from the experiment.

Transient Kinetics

All stopped-flow experiments were performed in 40 mM HEPES-KOH, pH 7.5, 100 mM NaCl, 10% glycerol at 4° C. NicA2 and CycN solutions were made anaerobic in glass tonometers by repeated cycles with vacuum and anaerobic argon⁴². When needed, NicA2's FAD was reduced in the anaerobic tonometer by titrating with a dithionite solution housed in a gas-tight Hamilton syringe. The dithionite solution was slowly added up to the point where NicA2's flavin reached the fully-reduced hydroquinone state, and the redox status of the flavin was spectrophotometrically monitored during the titration using a Shimadzu UV-1900 UV-VIS spectrophotometer. Nicotine-containing buffer solutions were made anaerobic by sparging for at least 10 minutes with anaerobic argon. Buffer containing O₂ at specific concentrations was prepared by sparging different O₂/N2 gas ratios through buffer in a gas-tight syringe for at least 15 minutes at room temperature. The various O₂/N2 gas ratios were prepared from O₂ and N2 gas cylinders using a Maxtec MaxBlend 2 gas mixer, and the dissolved O₂ concentration in the buffer solution was calculated using a Henry's law constant for O₂ of 770 atm M⁻¹.

Stopped-flow experiments were conducted using a TgK Scientific SF-61 DX2 KinetAsyst stopped-flow instrument that had been previously equilibrated with a glucose/glucose oxidase solution to make the internal components of the system anaerobic. About 30 μM NicA2 (concentration before mixing) was loaded onto the instrument and mixed with substrate (nicotine, O₂, or CycN) at a range of concentrations. The reaction of NicA2-Fl_{red} with O₂ was monitored using the instrument's multi-wavelength CCD detector (1.6 ms data interval time). The single wavelength detector was used for the reaction of NicA2-Flₒₓ with nicotine because the first phase in the reaction was too fast to get sufficient data coverage when using the CCD detector. The reaction of NicA2-Fl_{red} with CycN was monitored using both the CCD detector and the single wavelength detector, but only the single wavelength data were used for determining rate constants due to the superior data density of the single wavelength measurements. Kinetic traces were fit to sums of exponentials using KaleidaGraph (Synergy Software) to determine observed rate constants.

### Example 2

NicA2 Reacts Poorly with O₂

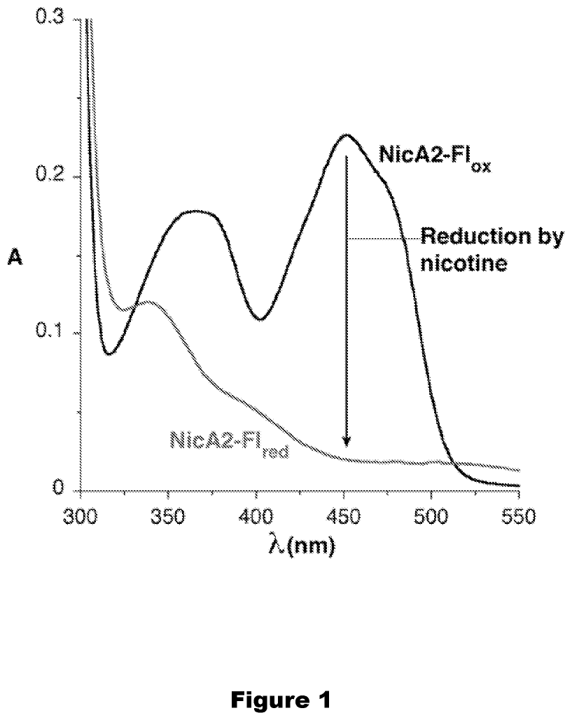

The flavin cofactor contained in the nicotine-degrading enzyme NicA2 provided a convenient spectrophotometric readout for NicA2's oxidation status simply by monitoring the UV-visible (UV-VIS) absorbance spectrum of this enzyme in the region of 300-500 nm′. In the absence of nicotine, NicA2 has an absorbance spectrum typical for oxidized FAD (FIG. 1). Upon the addition of 40 μM nicotine to 20 μM NicA2, however, a clear and sustained reduction of FAD to the two-electron reduced hydroquinone form (FADH₂) was seen, as indicated by a rapid decrease in absorbance in the 450 nm region. This rapid decrease occurred despite the presence of oxygen, which is present at about 250 μM in solutions under ambient conditions. NicA2's flavin remained in the FADH₂ state for several minutes before slowly re-oxidizing directly to oxidized FAD. This slow re-oxidation of NicA2 in the presence of oxygen indicated that flavin re-oxidation may be rate limiting during O₂-dependent nicotine turnover by NicA2.

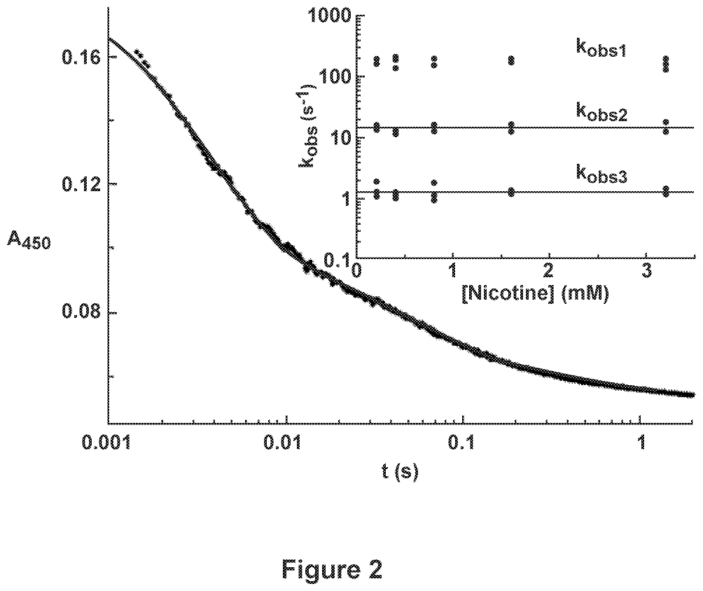

Like most flavin-dependent enzymes, the catalytic cycle of NicA2 can be divided up into half reactions. In the reductive half reaction, NicA2 containing oxidized FAD (NicA2-Flₒₓ) is reduced by reacting with nicotine to form N-methylmyosmine. In the oxidative half reaction, NicA2 containing FADH₂ (NicA2-Fl_{red}) is oxidized by reacting with O₂. To quantitatively define the extent that NicA2-Fl_{red} oxidation by O₂ limits the consumption of nicotine by NicA2, we decided to examine each of these individual half reactions by performing stopped-flow experiments. We first measured the kinetics of NicA2-Flₒₓ reduction by nicotine in the reductive half reaction under anaerobic conditions. Reduction of NicA2-Flₒₓ's FAD by nicotine was extremely fast (FIG. 2). Kinetic traces for this reaction required three exponentials in order to fit properly, indicating that at least three events occur when NicA2-Flₒₓ reacts with nicotine. The observed rate constant (k_{obs}) for each of the three phases did not change with increasing nicotine concentrations (FIG. 2 inset), indicating that all three events occur after the bimolecular step where nicotine first binds to NicA2-Flₒₓ. That the k_{obs} for all three phases did not increase as the nicotine concentration was raised also indicated that the K_{d} for nicotine binding is much lower than 100 μM, the lowest nicotine concentration used in the experiment. The fastest phase contributes the majority (65%) of the signal change at 450 nm where spectral changes correspond to FAD reduction, indicating that this phase reports on the reduction of NicA2's FAD by nicotine (and the corresponding formation of N-methylmyosmine). This step has a k_{obs} of about 180 s⁻¹ and is completed in about 20 ms, indicating that NicA2 rapidly oxidizes nicotine. The nature of the two subsequent events is unclear, but may involve conversion of N-methylmyosmine to pseudooxynicotine and/or product release. Notably, the entirety of the reductive half reaction is completed within 2 seconds.

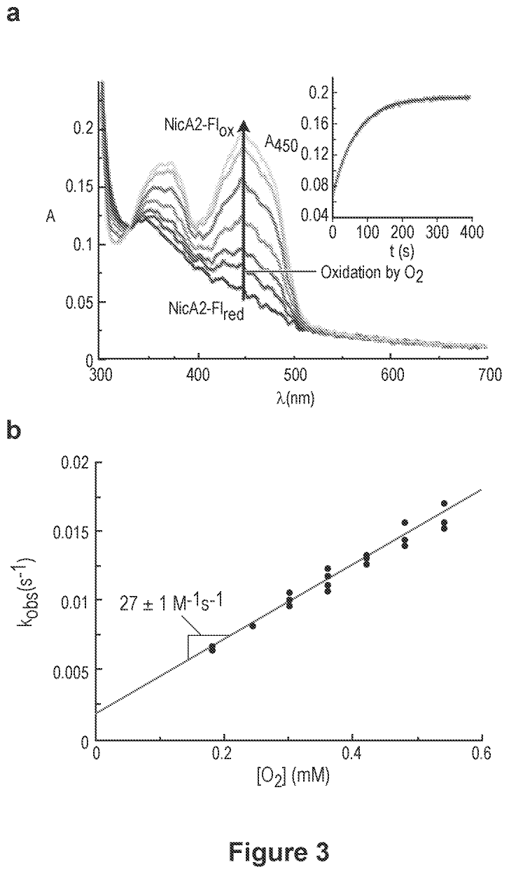

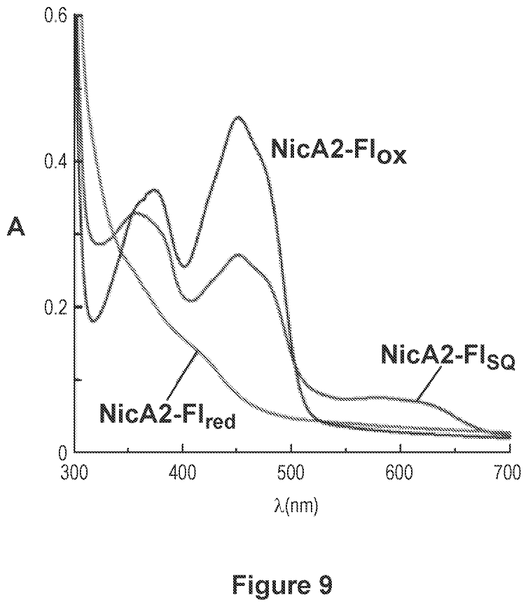

We next monitored the reaction of NicA2-Fl_{red} with O₂ in the oxidative half reaction via stopped-flow experiments. NicA2-Fl_{red} was made in an anaerobic glass tonometer by titrating NicA2-Flₒₓ with a dithionite solution until NicA2's FAD was fully reduced to FADH₂ (monitored using the absorbance spectrum of the flavin). Dithionite was used for reduction rather than nicotine to study the oxidative half reaction in the absence of any reaction products. During the dithionite titration, NicA2's FAD first populated a form with a UV-VIS spectrum resembling a neutral flavin semiquinone before reaching a spectrum consistent with the fully reduced FADH₂ state (FIG. 9). This observation indicates that NicA2's flavin is capable of performing one-electron transfers, given that populating a reduced semiquinone state is indicative of a one-electron transfer. Stable flavin semiquinones have been observed in several members of the flavin-dependent amine oxidase family of enzymes. Once fully reduced, NicA2-Fl_{red} was loaded on the anaerobic stopped-flow instrument and then mixed with buffer bubbled with various O₂/N₂ ratios to re-oxidize NicA2's flavin. Flavin re-oxidation by O₂ was dramatically slower than the reductive half-reaction with nicotine, taking about 400 seconds for complete re-oxidation of NicA2-Fl_{red} to NicA2-Flₒₓ at even the highest O₂ concentration used (540 μM). NicA2-Fl_{red} oxidized directly into NicA2-Flₒₓ without the formation of any intermediates (FIG. 3A). Kinetic traces at 450 nm could be fit with a single exponential and the observed rate constant for this phase increased linearly with O₂ concentration (FIG. 3A inset and 3B). This provides evidence for a bimolecular reaction in which O₂ directly reacts with NicA2-Fl_{red} to form NicA2-Flₒₓ. Fitting the plot of k_{obs} against O₂ to a line yielded a bimolecular rate constant for flavin oxidation (kₒₓ^{O2}) of 27±1 M⁻¹s⁻¹. At room temperature, the O₂ concentration in solution is ˜250 μM. At this concentration, NicA2-Fl_{red} oxidation by O₂ would occur with a k_{obs} of 0.007 s⁻¹. Strikingly, this k_{obs} exactly matches the 0.007 s⁻¹ turnover number reported in several studies for the in vitro O₂-dependent nicotine turnover by NicA2^{9,10} providing strong evidence that NicA2-Fl_{red} oxidation by O₂ is indeed rate-limiting for this reaction. The data thus indicate that O₂ is a rather poor electron acceptor for NicA2-Fl_{red}, especially when compared with most flavin-containing amine oxidases, which have a kₒₓ^{O2} between 10⁵-10⁶ M⁻¹s^{-1 49}.

### Example 3

Identification of the NicA2 Physiologic Electron Acceptor CycN

There is an obvious discrepancy between NicA2's slow turnover in vitro when using O₂ and P. putida S16's robust growth on nicotine. P. putida S16 is able to grow with a doubling time of about 90 minutes using nicotine as its sole carbon and nitrogen source⁷. At the established in vitro catalytic rate of NicA2 using O₂¹⁰, it is impossible to accumulate enough biomass from nicotine to sustain growth at this observed doubling time. Even if 5% of P. putida S16's biomass were to be made up of the NicA2 enzyme, we calculate that this enzyme must be functioning at a catalytic rate of at least 1 s⁻¹ in vivo to support a doubling time of 90 minutes (see Example 1, Estimating NicA2 turnover rate in vivo). This implies that NicA2 is catalyzing nicotine turnover faster in vivo than it does in vitro using O₂ as an electron acceptor, which indicates that NicA2 is likely using a different electron acceptor in vivo.

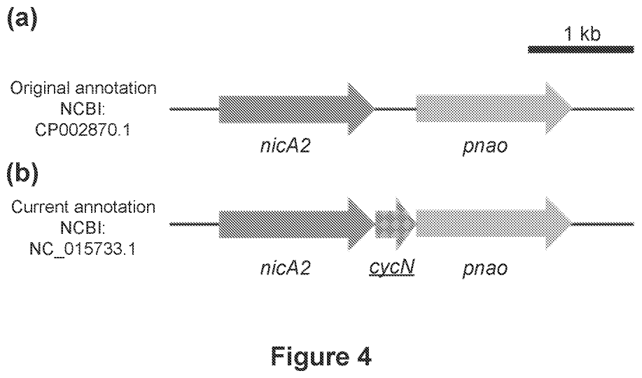

While searching the published genome of P. putida S16, we noticed that just downstream of nicA2, embedded within the same regulatory operon, is a poorly annotated open reading frame (PPS_RS28240) not previously noted in papers characterizing the nicotine degradation pathway (FIG. 4)^{6,7,16}. Importantly, the program SignalP 5.0¹⁷ predicted that both nicA2 and this open reading frame possess an N-terminal signal sequence for periplasmic localization, placing both proteins in the same cellular compartment. A homology search of the protein sequence encoded by this gene using NCBI BLAST revealed hits with highly significant e values (as low as 8e-68) to proteins annotated as cytochrome c proteins. We concluded that this gene encodes a cytochrome c protein, which we call CycN. The February 2020 update of the genome annotation in NCBI (NC_015733.1) concurs with our assignment of this open reading frame as a cytochrome c. Cytochromes c are small, soluble electron carrier proteins that are known to mediate electron transfer reactions, including the well-characterized transfer of electrons between complex III and complex IV of the electron transport chain during aerobic respiration²⁰. This observation, and the fact that cycN forms an operon with nicA2, led us to expect that CycN may play a similar role for NicA2, shuttling electrons between NicA2 and the electron transport chain of P. putida S16.

### Example 4

P. putida S16 ΔcycN Strain is Unable to Grow on Nicotine

If CycN is indeed the physiological electron acceptor for NicA2, there should be a significant growth phenotype on nicotine for a ΔcycN strain. To test this view, we generated an in-frame deletion of cycN in P. putida S16 and assessed this deletion for its ability to grow on nicotine¹⁸. Both the ΔcycN and wild-type (WT) strains grew similarly well on rich media (LB agar). When plated onto M9 salts agar supplemented with nicotine as the sole carbon and nitrogen source, however, there was a very clear phenotype for the ΔcycN strain (FIG. 5). While WT P. putida S16 formed large, easily visible colonies within two days, the ΔcycN strain grew extremely poorly. Presumably, this is because in the absence of CycN, the organism is forced to use other electron acceptors for NicA2, such as O₂. The low O₂-dependent activity of the enzyme is clearly not sufficient to support robust growth of P. putida S16 on nicotine. Notably, the nicotine growth phenotype of the ΔcycN strain could be complemented by plasmid-based expression of cycN. The ΔcycN strain containing pJN105-cycN grew on nicotine at a rate similar to WT (FIG. 5). These data indicate that the growth deficiency of the cycN deletion on nicotine is not due to a polar effect on genes downstream of cycN.

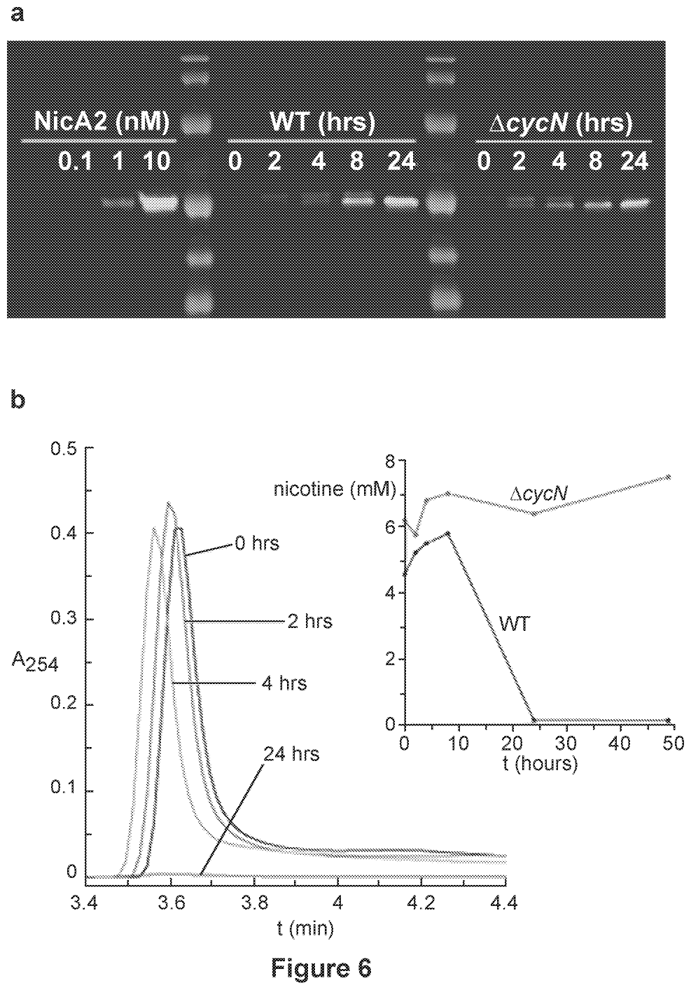

The growth phenotype of P. putida S16 ΔcycN on nicotine indicates that CycN is important for nicotine metabolism. To support the idea that this phenotype is due to a loss of NicA2's capacity to metabolize nicotine rather than, for instance, the absence of NicA2 itself in this strain, we sought to estimate the catalytic activity of NicA2 in vivo for both WT and P. putida S16 ΔcycN. To accomplish this, we used an approach that couples high-performance liquid chromatography (HPLC) with quantitative western blotting. WT and P. putida S16 ΔcycN cultures were first grown in minimal media with glycerol as the sole carbon source, and then supplemented with nicotine. At multiple time points after adding nicotine, culture samples were subjected to western blot analysis with an anti-NicA2 antibody and analyzed by HPLC to determine the concentration of nicotine remaining. The expression of NicA2 induced by nicotine was comparable in both strains (FIG. 6A). However, while the WT strain completely degraded all of the nicotine in the culture, the ΔcycN strain showed essentially no nicotine consumption over 24 hours (FIG. 6B). Because the NicA2 protein levels were similar in both strains, this likely indicated that NicA2's activity was dramatically reduced in the absence of CycN, in agreement with the expectation that CycN is the physiological electron acceptor for NicA2. By correlating the rate of nicotine turnover with the NicA2 protein level, we estimated the in vivo catalytic activity of NicA2 in WT P. putida S16: about 5 mM nicotine was entirely degraded by about 3.5 nM NicA2 in 16 hours, resulting in a turnover rate of about 24 s⁻¹. This in vivo rate is more than 3,000 times faster than the previously measured in vitro rate of 0.007 s⁻¹ when using O₂ as the electron acceptor. In reality, the turnover rate of the enzyme may be even more rapid when donating electrons to CycN, given that this approach looks at the entire pathway of nicotine degradation and is thus limited by the slowest step for nicotine degradation in the organism.

### Example 5

CycN is Reduced by NicA2

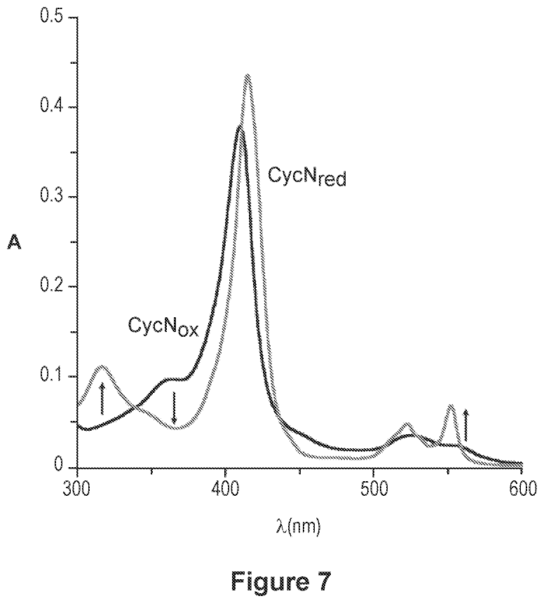

Flavin-dependent dehydrogenases are known to transfer their electrons to a variety of small molecules and protein clients, including cytochromes c^{19,20}. To further probe the relationship between NicA2 and CycN, we recombinantly expressed and purified both enzymes from E. coli and characterized their in vitro electron-transfer activities. Similar to how absorbance spectra provide a readout of flavin oxidation states, there are spectral signatures typically associated with the redox status for heme in cytochrome c as well²¹. We used these spectral differences as a readout to determine if NicA2 can transfer electrons from nicotine to CycN. Upon incubation of oxidized CycN with excess nicotine and a catalytic amount of NicA2, we observed a characteristic increase and shift of Soret banding pattern was observed, which indicated that CycN had become reduced (FIG. 7). This reduction in CycN required both nicotine and NicA2; omission of either of these components failed to reduce CycN.

### Example 6

NicA2-Fl_{red} is Rapidly Reoxidized by CycN

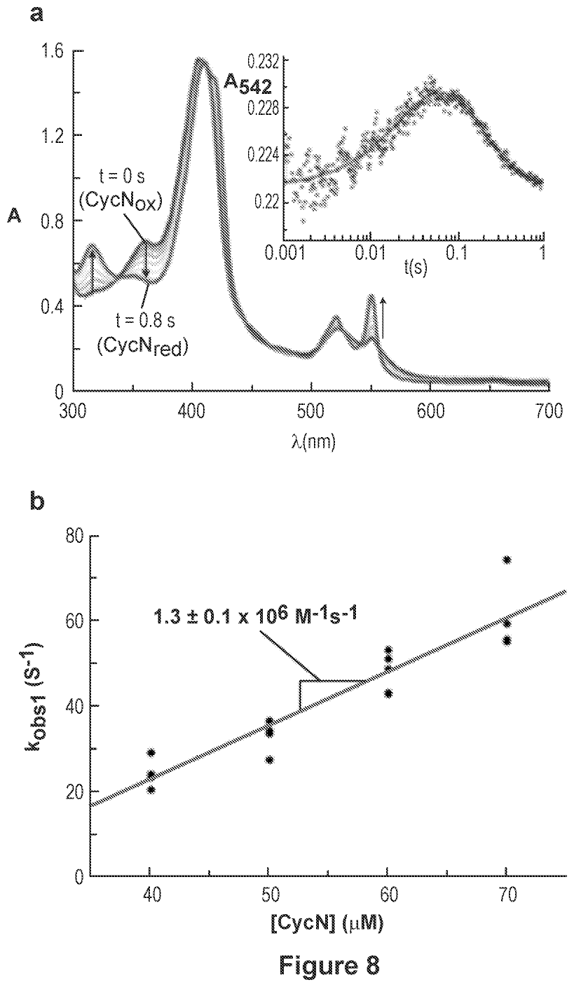

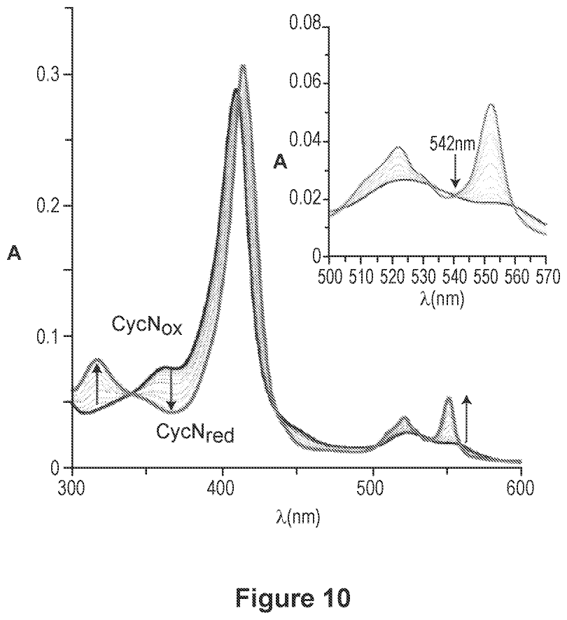

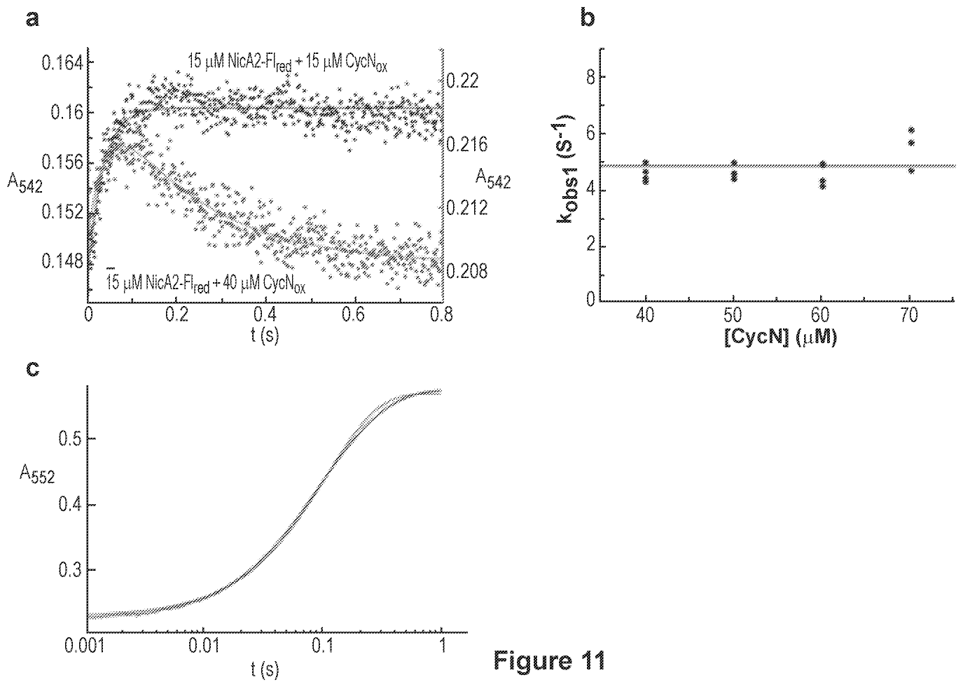

We next wanted to measure how fast electrons are transferred from NicA2-Fl_{red} to oxidized CycN (CycNₒₓ) in vitro. To determine this, we performed stopped-flow experiments by anaerobically mixing 15 μM NicA2-Fl_{red} with an excess of CycNₒₓ and monitored the changes in absorbance over time using the instrument's multi-wavelength detector. Electron transfer between NicA2-Fl_{red} and CycNₒₓ appeared to be very rapid, being complete in under one second. The absorbance changes were dominated by the signals associated with reduction of CycNₒₓ's heme from the Fe³⁺ to the Fe²⁺ state (CycN_{red}) (FIG. 8). Because of this, we analyzed the kinetics of the reaction at 542 nm. This wavelength was chosen for two reasons: (1) the absorbance of CycNₒₓ and CycN_{red} is identical at 542 nm (an isosbestic point; FIG. 10) and (2) NicA2's flavin semiquinone (NicA2-Fl_{SQ}) absorbs at this wavelength, but NicA2-Fl_{oX} and NicA2-Fl_{red} do not (FIG. 9). This wavelength allowed us to monitor the transient formation of NicA2-Fl_{SQ}, which would be expected to form since CycNₒₓ is an obligate one-electron acceptor and two CycNₒₓ molecules must react sequentially with NicA2-Fl_{red} in order to fully oxidize it back into NicA2-Flₒₓ. Kinetic traces of the reaction with more than a two-fold molar excess of CycNₒₓ indeed showed an increase, followed by a decrease in absorbance at 542 nm (FIG. 8). The amplitudes for the two phases were approximately the same (but opposite), which is again consistent with the formation of NicA2-Fl_{SQ} followed by its conversion to NicA2-Flₒₓ. We further tested whether the signal changes at 542 nm report on the presence of NicA2-Fl_{SQ} by mixing NicA2-Fl_{red} with an equimolar concentration of CycNₒₓ. Under these conditions, only the increase at 542 nm should be observed, but not the subsequent decrease, as there should only be enough CycNₒₓ to react once with NicA2-Fl_{red}. The kinetic trace from reacting 15 μM NicA2-Fired with 15 μM CycNₒₓ only showed the expected increase in absorbance at 542 nm without any subsequent decrease, confirming that we are observing formation of the NicA2-Fl_{SQ} at this wavelength (FIG. 11).

The stopped-flow traces at 542 nm using an excess of CycNₒₓ could be fit with a double exponential function. The k_{obs} for the first phase increased linearly with the CycNₒₓ concentration, indicating that this phase reports on the bimolecular association of NicA2-Fl_{red} with CycNₒₓ. Fitting the plot to a line yielded a bimolecular rate constant for the interaction of 1.3±0.2×10⁶ M⁻¹s⁻¹. Strikingly, this is about four orders of magnitude greater than the value for the oxidation of NicA2-Fl_{red} by O₂ (which is 27 M⁻¹s⁻¹). The fact that this phase is bimolecular indicates that electron transfer between CycNₒₓ and NicA2-Fl_{red} is rate-limited by the association between the two proteins; electron transfer between the two redox centers must therefore be extremely rapid after the complex has formed. The k_{obs} for the second phase appeared invariant at ˜5.5 s⁻¹ independent of the CycNₒₓ concentration used (FIG. 11). This indicates that the reaction of NicA2-Fl_{SQ} with the second CycNₒₓ molecule is rate-limited by some unimolecular event. This may be due to delayed dissociation of CycN_{red} from NicA2-Fl_{SQ} after reaction of the first CycNₒₓ with NicA2-Fl_{red}.

### Example 7

NicA2-Fl_{red} Reacts Poorly with Bovine Cytochrome c

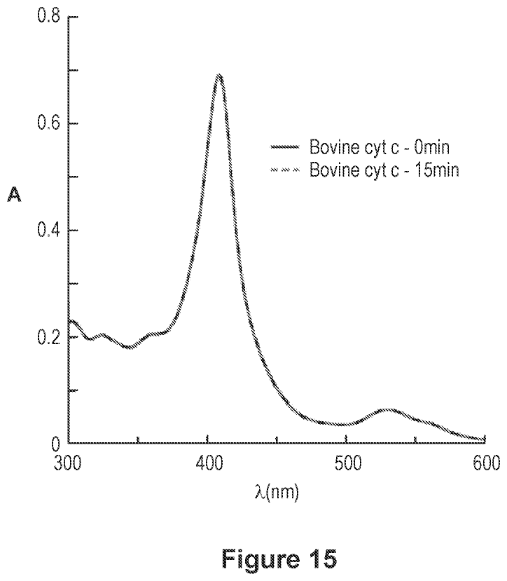

Our discovery that CycN is the physiological electron acceptor for NicA2 raised a question—is reactivity with NicA2 specific to CycN or will any cytochrome c perform this function? Bovine mitochondrial cytochrome c (43% sequence identity to CycN), a commonly used model protein for studying the function of the electron transport chain, was tested as an electron acceptor for NicA2. The bovine mitochondrial cytochrome c was not efficiently reduced by this system (FIG. 15), however, indicating that NicA2 is specific for CycN as an electron acceptor.

Disclosed herein is experimental evidence that NicA2 is a flavin-dependent dehydrogenase that uses CycN as its redox partner. Applying the lessons learned from NicA2 more generally, members of the flavin-dependent amine oxidase family are assumed to undergo oxidation by O₂ in vivo, and typically readily re-oxidize with molecular oxygen in vitro³. Without wishing to be bound by theory, the fact that NicA2 is able to transfer electrons to a cytochrome c protein prompts reconsideration of this generalized mechanism and raises the possibility that other members of this family use alternative physiologic electron acceptors. When amine oxidases use O₂ as a terminal electron acceptor, reactive oxygen species such as H₂O₂ or superoxide are released as a byproduct. Given the deleterious effect of these reactive oxygen species, it may be more desirable to shunt electrons down another path in vivo, as we have shown occurs with NicA2 in P. putida S16. Furthermore, just because an amine oxidase can rapidly be oxidized by O₂ in vitro does not necessarily mean that it uses O₂ as its electron acceptor in vivo. In this regard, there is recent evidence for the existence of one such example in mitochondria. Human monoamine oxidases (MAOs) A and B are prominent drug targets with important neuroregulatory functions. They are bound to the outer mitochondrial membrane by a transmembrane tail anchor, with different isoforms on either the cytosolic or intermembrane space leaflets²⁶⁻²⁸. The reason for this mitochondrial localization of monoamine oxidases has been an open question since their discovery. In their presumed catalytic cycle, MAOs are thought to be re-oxidized by molecular oxygen, producing H₂O₂ or other potentially damaging oxidants²⁹. Recent work, however, has demonstrated that human monoamine oxidases are not creating H₂O₂ as previously thought, and instead seem to be transferring electrons from amine oxidation to complex IV of the electron transport chain³⁰. Given that cytochrome c also exists in the mitochondrial intermembrane space, it may be the link facilitating electron transfer to the electron transport chain—bypassing the harmful reactive oxygen species produced when flavins are re-oxidized by O₂. For this reason, it is critical to re-evaluate the mechanistic paradigm of these enzymes. MAOs are frequent targets for in vitro drug studies, and it may be that they are deprived of vital redox cofactors in these studies, inaccurately skewing the result of turnover or inhibition assays.

### Example 8

NicA2 Protein Design and Directed Evolution

To increase the versatility and robustness of the disclosed technology for treating nicotine dependence and reducing relapse to nicotine dependence, protein design and directed evolution are used to modify NicA2 to yield an enzyme with improved pharmacokinetic and pharmacodynamic properties when using, e.g., O₂ as an electron acceptor. This approach is expected to yield therapies that are simple and cost-effective because only the modified NicA2 needs to be administered. Moreover, such therapies are valuable in situations where administration of alternative electron acceptors such as CycN or other cytochrome c proteins are contra-indicated.

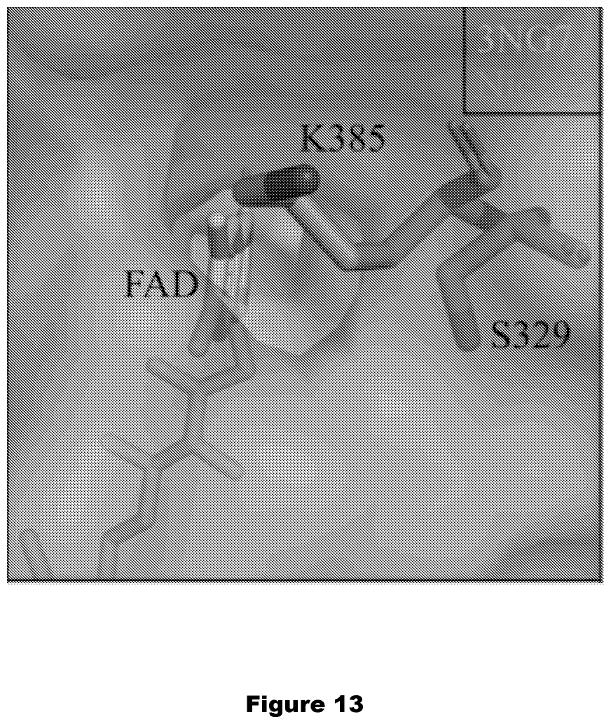

For protein design to be effective, we need to know how to manipulate the structure of the protein to alter its electron acceptor specificity. Nature frequently evolves the protein environment surrounding the flavin cofactor to tune its reactivity with oxygen and the evolutionary rules that govern this control are starting to be understood⁴⁹. Nicotine oxidase, given it very low k_{cat} with oxygen, appears to have evolved to suppress its ability to utilize oxygen as a final electron acceptor. Structural comparisons between nicotine oxidase and related enzymes will be used to understand why nicotine oxidase reacts so poorly with oxygen. For example, a simple comparison between nicotine oxidase and the structurally related L-6-hydroxynicotine oxidase (3NG7), which reacts with oxygen with a k_{cat} of 78/sec³⁸ (11,000 fold faster than NicA2 does), shows that a channel, which is conserved across several flavin-dependent oxidases and apparently allows oxygen access to the flavin adenine dinucleotide at the active site of 3NG7, is blocked by a lysine in nicotine oxidase (FIG. 13). Initially, two approaches are taken, i.e., opening up this channel so that NicA2 reacts more effectively with oxygen, and engineering hydroxynicotine oxidase so it reacts more effectively with the chemically very similar compound nicotine. Hydroxynicotine oxidase already has a 5-fold greater k_{cat} than NicA2 for degrading nicotine in an oxygen dependent reaction³⁸.

To assist us in altering the substrate specificity of these enzymes we will use directed evolution.⁵⁰⁻⁵⁴ Directed evolution has the advantage of not requiring the same detailed level of knowledge about the protein as protein design. It does, however, require a high throughput assay to rapidly screen a library of NicA2 mutants to find those that show enhanced activity in the presence of oxygen or screen through hydroxynicotine oxidase libraries to find those that can effectively utilize nicotine. To achieve this goal, we developed a genetic selection that exploits the fact that nicotine is toxic to E. coli. Libraries of engineered NicA2 or hydroxynicotine oxidase variants are transformed into E. coli and then selected for growth on a nicotine concentration where the activity of the wild-type enzyme is insufficient to allow growth. Plasmid DNA is isolated from the survivors, further mutated and, following re-transformation, subjected to second and third rounds of selection to further enhance nicotine resistance in the presence of oxygen. In addition to using nicotine toxicity as a genetic selection, NicA2 variants catalyzing the oxidation of nicotine using O₂ as electron acceptor can be selected in CycN⁻Pseudomonas putida S16, as described in greater detail below. In some embodiments, the CycN⁻ is a partial or complete deletion of the gene encoding CycN, while other embodiments involve at least one missense or nonsense mutation of the wild-type gene for CycN. Variants are characterized biochemically and tested in the C. elegans nicotine addiction model, as outlined below.³⁵

The nematode C. elegans is a popular genetic model for neuroscience research due to its small and well-annotated nervous system and amenability to genetic manipulation. It is known that C. elegans exhibits a variety of behavioral responses to nicotine, including acute response, adaptation, withdrawal, and sensitization.⁴⁷ Specifically, acute nicotine treatment stimulates locomotion. Repeated administration of nicotine sensitizes C. elegans to nicotine, and long-term treatment elicits tolerance to the drug. Nicotine-adapted worms exhibit hyperlocomotion when placed in a nicotine-free environment, a withdrawal response to nicotine. These nicotine responses require the same nicotinic acetylcholine receptors that are known to be critical for nicotine dependence in mammals. Thus, the behavioral responses induced by nicotine and the underlying molecular target of nicotine in C. elegans parallel those observed in mammals. These features, together with its short generation time, easy handling, and facile genetics make C. elegans a convenient model for studying nicotine dependence.

To test NicA2 and its variants in C. elegans, focus is placed on their effects on nicotine intoxication and withdrawal responses. NicA2 and its variants are expressed as transgenes in C. elegans and then acute nicotine responses and withdrawals are tested using standard protocols as described⁴⁷ As an initial test, NicA2 is expressed in all worm tissues using the ubiquitous promotor eft-3 or sur-5. It has previously been shown that nicotine acts on a group of command interneurons to exert its effect on worm locomotion⁴⁷ and references therein. Thus, the nmr-1 promoter is used to express NicA2 specifically in these command interneurons. Although nicotine can act both outside and inside the neurons, more efficient oxidation of nicotine by NicA2 may be achieved extracellularly. Therefore, a signal peptide is attached to NicA2 and the fusion is expressed as a secreted protein in worms to determine whether a secreted form works more efficiently. Alternatively, recombinant NicA2 and its variants are injected into the worm body cavity by microinjection. If the electron acceptor is a small molecule, its effect is tested by microinjection, provided in the worm media or within the bacteria used to feed the worm. Because E. coli is commonly used to feed C. elegans this opens up the possibility of using various genetically manipulated strains of E. coli that contain either small molecules or proteins. If the electron acceptor is a peptide, it can also be expressed as a transgene in the worm. Thus, a platform to test the ability of various NicA2 variants to antagonize nicotine dependence in an in vivo setting is provided. In accordance with the technology disclosed herein, a combination of classical flavin biochemistry and laboratory evolution is used to first enhance the utility of a promising nicotine dependence treatment and then to verify that it works in a worm model.

Consistent with the foregoing description, the disclosure provides materials and methods relating to NicA2 variants able to efficiently use O₂ as electron acceptor and to variants of the structurally related LHNO flavin enzyme able to oxidize nicotine. The protein matrix surrounding NicA2's FAD is suppressing the innate ability of FAD to react with O₂. Notably, NicA2 belongs to the monoamine oxidase structural family of flavin-dependent enzymes; members of this class of enzyme usually react very rapidly with O₂, making NicA2 an anomaly among the monoamine oxidase family. For example, L-6-hydroxynicotine oxidase (LHNO), which has structural and sequence (30% identity) homology to NicA2 (FIG. 12A, 12B), has a k_{cat} of 78 s⁻¹ when using O₂ as the electron acceptor for flavin oxidation³⁸. This makes the k_{cat} for LHNO more than 10,000 times faster than that of NicA2 when using O₂, even though the substrates—L-nicotine and L-6-hydroxynicotine—for the two enzymes differ by a single additional hydroxyl group. This fact, and the high homology between the two enzymes, leads to the expectation that the reactivity of NicA2 towards O₂ can be enhanced through protein engineering by focusing on the key structural features responsible for NicA2's low reactivity towards O₂. Subtle factors in the vicinity of a flavin cofactor like charge, hydrophobicity and pre-formed O₂ reaction sites can enhance its reactivity with O₂^{25,49,60,61.} Indeed, many of the residues surrounding the FAD cofactor in NicA2 are the same, or similar, to those in LHNO, which reacts rapidly with O₂ (FIG. 12C, 12D). This indicates that the poor reactivity with O₂ in NicA2 may not be due to the local biochemical features of the FAD binding site, but might instead involve distant properties like protein dynamics or access of the FAD cofactor to solvent. In addition to rational protein design, a directed evolution approach (described herein is used to elucidate the structural features responsible for limiting NicA2's reactivity with O₂. The fact that both NicA2 and LHNO have solved structures is advantageous both in directing evolution and in determining the mechanism behind performance-enhancing mutations.

Nicotine Toxicity and Genetic Mutations as Selective Pressures

Directed evolution is a powerful means to improve enzyme function. By randomly mutating an enzyme and then selecting for improved performance⁶², we can exploit the principles of evolution to rapidly generate a variant of NicA2 with drastically improved O₂-dependent activity. The experiments are performed using the fast-growing model organism, Escherichia coli, which permits evaluation of many variants of NicA2 for performance-enhancing mutations. We have established that nicotine is toxic to E. coli at a level of about 1 mg/mL or higher. We have also determined that pseudooxynicotine, the product from nicotine oxidation catalyzed by NicA2, is non-toxic to E. coli up to at least 5 mg/mL. This effectively makes nicotine an antibiotic against E. coli. Thus, a vector borne copy of the nicA2 coding region is mutagenized using any known technique for mutagenesis and the mutant library is transformed into E. coli, where selection is applied by culturing the transformed cells on media containing at least 1 mg/mL nicotine. NicA2 variant expression should confer resistance to nicotine toxicity that is dependent on the O₂-dependent performance of the NicA2 variant. This nicotine toxicity towards E. coli provides a selection to identify NicA2 variants with enhanced nicotine oxidase activity. Identification of these variants is expected to reveal the molecular determinants of wild-type NicA2's low O₂-dependent activity.

In addition to performing a selection in E. coli based on toxicity of nicotine to this organism, we can also use a genetically modified P. putida S16 ΔnicA2 ΔcycN or ΔcycN alone strain to accomplish selection of O₂-utilizing nicA2 variants. When P. putida S16 ΔnicA2 ΔcycN or ΔcycN alone is grown on media containing nicotine as its sole carbon and/or nitrogen source, growth is directly limited by the ability of the organism to degrade nicotine. Upon transforming this strain with a vector-borne copy of nicA2, the organism may grow only as quickly as reaction of NicA2 with O₂ allows, as CycN is not available in the knockout strain to accept electrons from nicotine oxidation. Wild-type nicA2 results in very slow growth (FIG. 5) in the P. putida S16 ΔcycN strain as it is limited by wild-type NicA2's very poor activity with O₂. When a mutant library of vector-borne nicA2 variants, as referred to above, is transformed into the strain, however, variants with improved O₂-dependent nicotine-degrading activity will confer a growth advantage to their host allowing it to grow rapidly in comparison to cells containing wild-type or worse-performing variants. Thus, we can detect such improved variants by transforming P. putida S16 ΔnicA2 ΔcycN or ΔcycN alone with a vector-borne nicA2 mutant library, culturing on agarose plates containing nicotine as a sole carbon and/or nitrogen source, then looking for the largest colonies which indicate that they are most rapidly degrading nicotine. These colonies are then isolated and sequenced to determine the variants of nicA2 that are most active with O₂. A similar selection is performed in liquid media with nicotine as a sole carbon and/or nitrogen source.

Another strategy for developing nicotine cessation therapeutics employs a directed evolution approach to enhance the capacity of L-6-hydroxynicotine oxidase to use nicotine as a substrate, using similar selections. Mutagenesis of a coding region for LHNO followed by transformation into E. coli or P. putida S16 ΔnicA2 ΔcycN or ΔcycN alone and selection on nicotine medium is expected to result in LHNO variants able to oxidize nicotine using O₂, the native electron acceptor for LHNO-catalyzed redox reactions.

As disclosed above, the poor O₂ reactivity of NicA2 is addressed using directed evolution from two different angles—engineering NicA2 to react better with O₂ and evolving L-6-hydroxynicotine oxidase (LHNO) to use nicotine as a substrate. Both strategies are expected to generate a more potent O₂-dependent nicotine-degrading enzyme useful in tobacco-cessation therapies. For NicA2, the selective pressure of nicotine toxicity on E. coli is exploited to objectively interrogate the importance of all components of the enzyme on its ability to react with O₂. For LHNO, the increase in UV absorbance (e.g., absorbance at 280-300 nm) that occurs when nicotine is converted to pseudooxynicotine is used as the readout in an evolutionary screen. The screen will allow us to probe the importance of individual residues involved in substrate binding on the reactivity with O₂, as nicotine binding and O₂ reactivity may be inversely linked. This approach will identify how natural selection has suppressed the reactivity of NicA2's FAD with O₂, even though it belongs to the monoamine oxidase structural family of enzymes that usually react rapidly with O₂.

Rather than relying on potentially imperfect guidance provided by comparing structures, every component of NicA2's 445-amino-acid sequence (mature size) is evaluated for its impact on suppressing the reactivity of NicA2's FAD with O₂. This requires a high-throughput assay that can be used to rapidly screen through NicA2 mutants to identify ones that react rapidly with O₂. To avoid enhancing the reactivity with O₂ at the expense of reacting with nicotine, the inherent toxicity of nicotine towards E. coli is used as a selective readout in order to screen through large numbers of NicA2 variants for the ability to react rapidly with both O₂ and nicotine. Notably, E. coli's aerobic respiratory chain lacks a diffusible cytochrome c, instead using quinones to link the flow of electrons from dehydrogenases to terminal oxidases. This fact reduces the possibility that NicA2 variants are identified in the selection that couple nicotine oxidation to the electron transport chain instead of using O₂ as the electron acceptor. The GeneMorph II Random Mutagenesis Kit (Agilent®) and passage through the XL-1 Red E. coli mutator strain (Agilent®) are used as parallel, independent strategies for generating a library of NicA2 mutants in a pBAD expression vector (Copp et al., 2014; Guzman et al., 1995). Both methods of generating random mutant libraries are embodied in commercially available kits. The library of NicA2 expression constructs is then transformed into E. coli and selected for growth on concentrations of nicotine under aerobic conditions where the wild-type enzyme is unable to detoxify the nicotine and allow for growth. Plasmids from colonies that survive are isolated and retransformed into a clean strain background to verify that the enhanced nicotine tolerance is specifically due to the NicA2 variant and not mutations in the host background. After subsequently identifying the performance-enhancing mutation(s), they are run through additional rounds of mutagenesis and selection on even higher nicotine concentrations until the most potent O₂-dependent nicotine-consuming NicA2 variant is reached. Variants that confer improved nicotine resistance on E. coli are purified and characterized by stopped-flow analyses to quantitatively determine the improvement in their performance.

Evolve LHNO to Use Nicotine as a Substrate

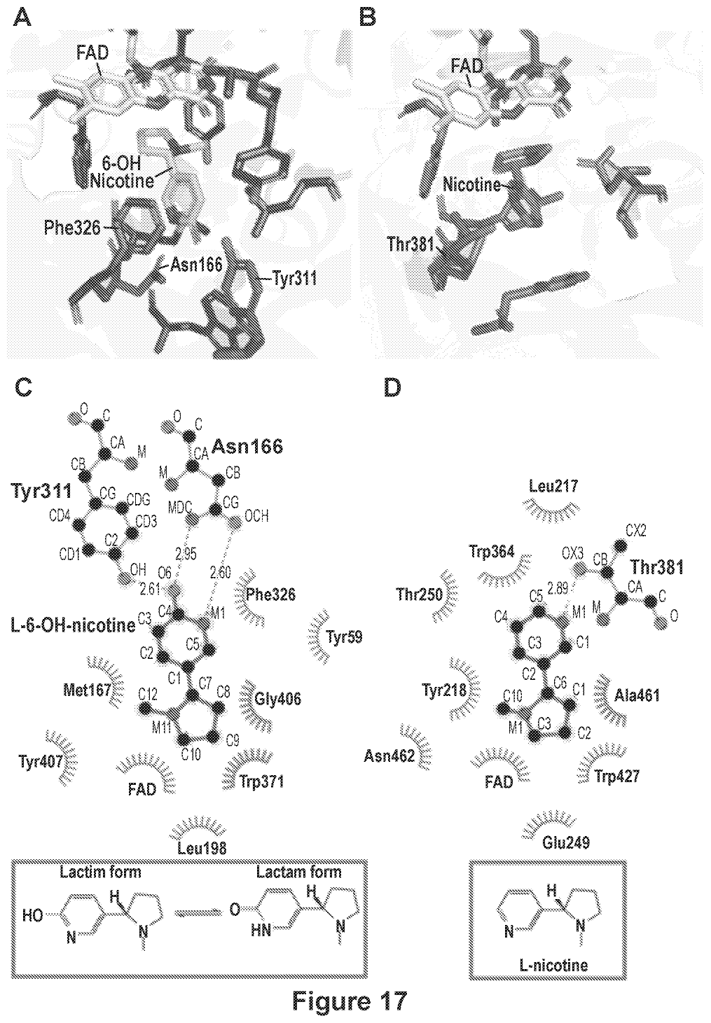

NicA2 and LHNO, with highly similar substrates, reactions, and structures, nevertheless have drastically different rates of reacting with O₂. While the amino acid environments of the FAD prosthetic groups are highly similar for the two enzymes, their substrate binding sites have a number of differences that enable them to discriminate between L-nicotine and L-6-hydroxynicotine (FIG. 17A, 17B). These substrate binding site differences raise the possibility that some of the residues required for nicotine binding in NicA2 may be preventing the enzyme from being able to react rapidly with O₂, i.e., efficient reaction with nicotine and O₂ may be mutually exclusive (for example, a residue important for recognizing nicotine may inhibit access of O₂ to the isoalloxazine of FAD). Because LHNO has already solved the problem of being able to react rapidly with O₂, directed evolution is used to enhance the substrate promiscuity of LHNO so that it reacts more readily with nicotine. If the ability to react with nicotine and O₂ are inversely linked, an increase in LHNO specificity towards nicotine is expected to produce an enzyme with lower reactivity towards O₂, which is evaluated using stopped-flow experiments as disclosed herein. If not, LHNO's specificity for nicotine is expected to be amenable to increase without affecting the enzyme's ability to react with O₂. Notably, wild-type LHNO has already been shown to slowly react with nicotine (k_{cat}/Kₘ of 0.042 mM⁻¹s⁻¹ for L-nicotine versus 600 mM⁻¹s⁻¹ for L-6-hydroxy-nicotine)³⁸, which is an important criterion before attempting to enhance specificity towards nicotine through directed evolution.

A comparison of the substrate-protein interaction maps of LHNO and NicA2 reveals how LHNO may discriminate between L-6-hydroxynicotine and L-nicotine (FIG. 17C, 17D). Asn166 and Tyr311 in LHNO form strong hydrogen bonds with the pyridine oxygen and nitrogen of L-6-hydroxynicotine. Given the hydrogen bonding pattern of these two residues in the active site, L-6-hydroxynicotine is likely present as the lactam tautomer in LHNO; this provides some additional discriminating power against L-nicotine since the carbonyl of Asn166 that hydrogen bonds with the protonated pyridine nitrogen of L-6-hydroxynicotine is unable to do so with the unprotonated pyridine nitrogen of L-nicotine. In NicA2, these two residues are replaced by the more hydrophobic Leu217 and Trp364, which should be better at accommodating the more nonpolar pyridine of L-nicotine. NicA2 also has Thr381 in the active site that helps position L-nicotine through a hydrogen bond with the pyridine nitrogen; LHNO has Phe326 at this position, which stacks with the pyridine ring and actually causes the substrate to bind with a different rotation angle around the pyridine-pyrrolidine bond than in NicA2. Therefore, these three residues of LHNO—Asn166, Tyr311 and Phe326—are expected to be useful focal points for targeted mutagenesis to enhance LHNO's specificity towards L-nicotine. Site-saturation mutagenesis by overlap extension PCR is used to generate a library of LHNO mutants with amino acid diversity both individually and in combination at these three residues (Williams et al., 2014). Pseudooxynicotine, the product from nicotine oxidation, absorbs UV light in the 280-300 nm range, whereas nicotine does not (Tang et al., 2013). After expressing the library of LHNO mutants in E. coli, this increase in absorbance is used as the readout for nicotine oxidase activity in a 96-well plate-based high-throughput screening assay to identify strains that express LHNO variants with increased specificity towards nicotine. Nicotine toxicity is not the initial readout in the initial search because of the risk that it could miss LHNO mutations that confer only incremental improvements in nicotine oxidase activity, mutations that may be mechanistically informative. Beyond targeting these three residues, libraries of mutants at other substrate binding site positions are evaluated using the nicotine toxicity selection described herein for NicA2.

Additionally, if CycN is, as expected, the physiological electron acceptor, then a ΔnicA2ΔcycN double deletion strain of P. putida S16 is used as the host organism in a selection to identify NicA2 variants with improved O₂-dependent activity. ΔnicA2ΔcycN P. putida S16 expressing wild-type NicA2 from a plasmid will be unable to grow, or will grow poorly because nicotine turnover should only be possible by using O₂ as the electron acceptor in this strain when cultured on media containing nicotine as the sole carbon and nitrogen source. NicA2 variants expressed from the plasmid-based library with an enhanced ability to use O₂ are identifiable as displaying faster growth and a correspondingly larger colony size on nicotine media plates. Another experimental strategy takes advantage of a recently developed GFP-based nicotine biosensor⁵⁸. This biosensor specifically recognizes nicotine, displaying low fluorescence in the absence of nicotine and high GFP fluorescence when bound to nicotine. The library of NicA2 variants in E. coli is co-expressed with this nicotine biosensor in the presence of nicotine and then fluorescence-activated cell sorting is used to rapidly screen through the library of NicA2 expressers to identify cells with lower GFP-fluorescence, corresponding to higher nicotine oxidase activity. Yet another experimental strategy makes use of the same readout that used for enhancing the nicotine specificity of LHNO, i.e., the increase in UV absorbance that accompanies the conversion of nicotine to pseudooxynicotine. This spectrophotometric readout is used, e.g., in 96-well plate assays. To accommodate the relatively low throughput of this assay, mutagenesis libraries may be focused on targeted NicA2 residues in the active site and those that line solvent-access channels (identified using CAVER; Jurcik et al., 2018).

The experiments are expected to identify the structural features of NicA2 that are responsible for restricting its FAD's ability to react with O₂. Furthermore, we expect to identify LHNO variants that are potent nicotine oxidase enzymes without compromising the ability of the wild-type enzyme to react rapidly with O₂. The performance-enhanced enzyme variants overcome the limitations of previously developed enzyme-based tobacco-cessation therapies—namely, the excessively high doses of enzyme needed due to the low O₂-dependent nicotine oxidase activity of the wild-type enzymes. The enzyme variants are expected to result in enzyme-based tobacco-cessation therapeutics.

### Example 9

NicA2 Protein Variants

To increase the versatility and robustness of the disclosed technology for treating nicotine dependence and reducing relapse to nicotine dependence, protein design and directed evolution were used to modify NicA2 to yield an enzyme with improved pharmacokinetic and pharmacodynamic properties when using, e.g., O₂ as an electron acceptor. This approach yielded therapies that are simple and cost-effective because only the modified NicA2 needs to be administered. Moreover, such therapies are valuable in situations where administration of alternative electron acceptors such as CycN or other cytochrome c proteins are contra-indicated.

For protein design to be effective, we needed to know how to manipulate the structure of the protein to alter its electron acceptor specificity. Nature frequently evolves the protein environment surrounding the flavin cofactor to tune its reactivity with oxygen and the evolutionary rules that govern this control are starting to be understood⁴⁹. Nicotine oxidoreductase, given its very low k_{cat} with oxygen, appears to have evolved to suppress its ability to utilize oxygen as a final electron acceptor. Structural comparisons between nicotine oxidoreductase and related enzymes was used to understand why nicotine oxidoreductase reacts so poorly with oxygen. For example, a simple comparison between nicotine oxidoreductase and the structurally related L-6-hydroxynicotine oxidase (3NG7), which reacts with oxygen with a k_{cat} of 78/sec³⁸ (11,000 fold faster than NicA2 does), showed that a channel, which is conserved across several flavin-dependent oxidases and apparently allows oxygen access to the flavin adenine dinucleotide at the active site of 3NG7, is blocked by a lysine in nicotine oxidase (FIG. 13). Initially, two approaches were taken, i.e., opening up this channel so that NicA2 reacts more effectively with oxygen, and determining other residues of importance to oxygen reactivity of NicA2 using random mutagenesis and a genetic selection described herein.

To assist us in altering the substrate specificity of this enzyme we have used directed evolution.⁵⁰⁻⁵⁴ Directed evolution has the advantage of not requiring the same detailed level of knowledge about the protein as protein design. It does, however, require a high throughput assay to rapidly screen a library of NicA2 mutants to find those that show enhanced activity in the presence of oxygen. To achieve this goal, we developed a genetic selection that exploits the fact that nicotine is toxic to E. coli. Libraries of engineered NicA2 variants are transformed into E. coli and then selected for growth on a nicotine concentration where the activity of the wild-type enzyme is insufficient to allow growth. Plasmid DNA was isolated from the survivors, further mutated and, following re-transformation, subjected to second and third rounds of selection to further enhance nicotine resistance in the presence of oxygen. In addition to using nicotine toxicity as a genetic selection, NicA2 variants catalyzing the oxidation of nicotine using O₂ as electron acceptor were selected in CycN⁻ Pseudomonas putida S16, as described in greater detail below. In some embodiments, the CycN⁻ was a partial or complete deletion of the gene encoding CycN, while other embodiments involved at least one missense or nonsense mutation of the wild-type gene for CycN. Variants were characterized biochemically.

Consistent with the foregoing description, the disclosure provides materials and methods relating to NicA2 variants able to efficiently use O₂ as electron acceptor. The protein matrix surrounding NicA2's FAD is suppressing the innate ability of FAD to react with O₂. Notably, NicA2 belongs to the monoamine oxidase structural family of flavin-dependent enzymes; members of this class of enzyme usually react very rapidly with O₂, making NicA2 an anomaly among the monoamine oxidase family. For example, L-6-hydroxynicotine oxidase (LHNO), which has structural and sequence (30% identity) homology to NicA2 (FIG. 12A, 12B), has a k_{cat} of 78 s⁻¹ when using O₂ as the electron acceptor for flavin oxidation³⁸. This makes the k_{cat} for LHNO more than 10,000 times faster than that of NicA2 when using O₂, even though the substrates— L-nicotine and L-6-hydroxynicotine—for the two enzymes differ by a single hydroxyl group. This fact, and the high homology between the two enzymes, leads to the expectation that the reactivity of NicA2 towards O₂ can be enhanced through protein engineering by focusing on the key structural features responsible for NicA2's low reactivity towards O₂, and the data disclosed herein reveal that this expectation has been realized. Subtle factors in the vicinity of a flavin cofactor like charge, hydrophobicity and pre-formed O₂ reaction sites can enhance its reactivity with O₂^{25,49,60,61}. Indeed, many of the residues surrounding the FAD cofactor in NicA2 are the same, or similar, to those in LHNO, which reacts rapidly with O₂ (FIG. 12C, 12D). This indicates that the poor reactivity with O₂ in NicA2 may not be due to the local biochemical features of the FAD binding site, but might instead involve distant properties like protein dynamics or access of the FAD cofactor to solvent. In addition to rational protein design, a directed evolution approach (described herein) is used to elucidate the structural features responsible for limiting NicA2's reactivity with O₂. The fact that both NicA2 and LHNO have solved structures is advantageous both in directing evolution and in determining the mechanism behind performance-enhancing mutations.

Nicotine Toxicity and Genetic Mutations as Selective Pressures

Directed evolution is a powerful means to improve enzyme function. By randomly mutating an enzyme and then selecting for improved performance⁶², we exploited the principles of evolution to rapidly generate a variant of NicA2 with drastically improved O₂-dependent activity. The experiments were performed using the fast-growing model organism, Escherichia coli, which permitted evaluation of many variants of NicA2 for performance-enhancing mutations. We have established that nicotine is toxic to E. coli at a level of about 1 mg/mL or higher. We have also determined that pseudooxynicotine, the product from nicotine oxidation catalyzed by NicA2, is non-toxic to E. coli up to at least 5 mg/mL. This effectively makes nicotine an antibiotic against E. coli. Thus, a vector-borne copy of the nicA2 coding region is mutagenized using any known technique for mutagenesis and the mutant library is transformed into E. coli, where selection is applied by culturing the transformed cells on media containing at least 1 mg/mL nicotine, yielding NicA2 variants able to survive the selection, as shown by the results disclosed herein. NicA2 variant expression should confer resistance to nicotine toxicity that is dependent on the O₂-dependent performance of the NicA2 variant. This nicotine toxicity towards E. coli provides a selection to identify NicA2 variants with enhanced nicotine oxidase activity. Identification of these variants is expected to reveal the molecular determinants of wild-type NicA2's low O₂-dependent activity.

In addition to performing a selection in E. coli based on toxicity of nicotine to this organism, we also used a genetically modified P. putida S16 ΔnicA2 ΔcycN or ΔcycN alone strain to accomplish selection of O₂-utilizing nicA2 variants. When P. putida S16 ΔnicA2 ΔcycN or ΔcycN alone was grown on media containing nicotine as its sole carbon and/or nitrogen source, growth was directly limited by the ability of the organism to degrade nicotine. Upon transforming this strain with a vector-borne copy of nicA2, the organism may grow only as quickly as reaction of NicA2 with O₂ allows, as CycN is not available in the knockout strain to accept electrons from nicotine oxidation. Wild-type nicA2 resulted in very slow growth (FIG. 5) in the P. putida S16 ΔcycN strain as it is limited by wild-type NicA2's very poor activity with O₂. When a mutant library of vector-borne nicA2 variants, as referred to above, was transformed into the strain, however, variants with improved O₂-dependent nicotine-degrading activity conferred a growth advantage to their host allowing it to grow rapidly in comparison to cells containing wild-type or worse-performing variants (FIG. 18). Thus, we detected such improved variants by transforming P. putida S16 ΔnicA2 ΔcycN or ΔcycN alone with a vector-borne nicA2 mutant library, culturing on agarose plates containing nicotine as a sole carbon and/or nitrogen source, then looking for the largest colonies which indicated that they were most rapidly degrading nicotine. These colonies were then isolated and sequenced to determine the variants of nicA2 that were most active with O₂. A similar selection was performed in liquid media with nicotine as a sole carbon and/or nitrogen source. The resulting NicA2 protein variants are disclosed in Table 2, along with each amino acid variation from the wild-type NicA2 amino acid sequence of SEQ ID NO:131 found in each of the NicA2 protein variants.

TABLE 2

| NicA2 variant | SEQ ID NO | Amino acid substitutions (single-letter code) |
|---|---|---|
| Nica2mut1 | 35 | D243N¹, K314Q, H368R |
| NicA2mut5 | 36 | W427L, A443V |
| NicA2mut6 | 67 | G62S, Q75H, R78G, T92S, H368R |
| NicA2mut7 | 90 | D130V, V147L, G156S, T267S, G411D |
| NicA2mut8 | 44 | R96S, D130G |
| NicA2mut9 | 63 | D130V, I161V, E188D |
| NicA2mut10 | 77 | G50D, T92I, L480P, L481A, S482E, 483L, 484I |
| NicA2mut11 | 51 | A107T, P177L, K292Q |
| NicA2mut12 | 45 | H115Y, T258A |
| NicA2mut17 | 68 | G29S, F104I, T223N, M265I |
| NicA2mut19 | 52 | C72S, L217P, V308L |
| NicA2mut20 | 53 | R85C, P114Q, G411D |
| NicA2mut21 | 40 | S37N, Q359R, H368R |
| NicA2mut22 | 59 | D130G, G431D, A461V |
| NicA2mut23 | 75 | T94A, P131S, A281T, G302A |
| NicA2mut25 | 76 | D130E, D224E, R351H, G431S |
| NicA2mut31 | 50 | Q75H, A429V |
| NicA2mut35 | 89 | T138I, F174I, D295V, H368Y, T455A |
| NicA2mut36 | 32 | T39S |
| NicA2mut40 | 60 | T133I, L135M, G474S |
| NicA2mut43 | 41 | W364C, Q442H |
| NicA2mut45 | 49 | Q75H |
| NicA2mut61 | 48 | R253S, K337M |
| NicA2mut64 | 79 | E172G, G338S, V390I, H460L |
| NicA2mut65 | 91 | S46R, A107P, V277I, I298F, E476V |
| NicA2mut66 | 46 | T381I, A444G |
| NicA2mut75 | 34 | D130N |
| NicA2mut95 | 96 | F63V, R96H, D130N, V277I, S379N, L480P, L481A, |
| | | S482E, 483L, 484I |
| NicA2mut96 | 33 | H368R, L435Q |
| NicA2mutD1 | 23 | G145A, S192I, Q359L, Q366R, S379N |
| NicA2mutH3 | 24 | R96S, F104L, M120I, H368R |
| NicA2mutH4 | 22 | P131S, K225Q, A461V |
| NicA2mut101 | 64 | A45T, Q366K, I380V |
| NicA2mut105 | 65 | E129Q, D130N, S151I, R160H |
| NicA2mut106 | 42 | G287D, R351H |
| NicA2mut112 | 47 | A98E, D130A |
| NicA2mut113 | 43 | E103D, E184K |
| NicA2mut117 | 78 | P180L, A244V, T258A, N462S |
| NicA2mut118 | 62 | K137R, L217P, N457T |
| NicA2mut119 | 69 | S276N, K292N, K324E, R351H |
| NicA2mut123 | 61 | Q75H, P131S, P152S, I159V |
| NicA2mut130 | 70 | P131S, V277I, V282I |
| NicA2mut136 | 54 | G50C, R393C |
| NicA2mut137 | 92 | V48A, F104L, D130Y, T267I, L435Q |
| NicA2mut138 | 37 | S146G, H368R, V432M |
| NicA2mut144 | 93 | D130G, P131Q, S192N, M306V, A473V |
| NicA2mut145 | 81 | D130G, K157M, V173A, K191M |
| NicA2mut146 | 82 | R85H, P229L, R351H, R437H |
| NicA2mut147 | 84 | G156C, T267S, W427L |
| NicA2mut149 | 31 | H368R |
| NicA2mut152 | 83 | T267I, W427L |
| NicA2mut155 | 38 | D130G, S379N |
| NicA2mut158 | 71 | W108R, D130Y, T183I, M403I |
| NicA2mut160 | 94 | T39S, K157R, I161F, M246T, H368L, E373K |
| NicA2mut163 | 55 | L135M, H368R, L449V |
| NicA2mut167 | 85 | D130G, D196H, M246T, K331N |
| NicA2mut169 | 72 | G50A, L217P, G260A, V345L |
| NicA2mut173 | 66 | D130N, V303A, N311S, A395V |
| NicA2mut174 | 80 | T42A, R78H, D130V, K225N, F245Y |
| NicA2mut175 | 39 | F104L, G317D, H368R |
| NicA2mut177 | 21 | G145A, S192I, Q359L, Q366R, S379N |
| NicA2mut179 | 86 | F104L, D130G, G270C, R393C |
| NicA2mut180 | 95 | G156D, H168Q, K292R, K324E, R351H |
| NicA2mut183 | 73 | W108R, D130Y, K337M, R398L |
| NicA2mut189 | 56 | G333S, R351H, L424Q |
| NicA2mut191 | 97 | F104I, D130G, G156D, K199R, D295N, T381S |
| NicA2mut192 | 87 | G99D, D130V, E454D |
| NicA2mut194 | 57 | F174V, G260S, W427L |
| NicA2mut198 | 74 | E249G, H368R |
| NicA2mut201 | 88 | D130G, R187L, V231I, Y242N, E408D |
| NicA2mut202 | 58 | A45V, D130V, I388F |
| NicA2mut204 | 30 | A107T, D130V, I330T |
| NicA2mut208 | 29 | G411D, W427L |
| NicA2mut210 | 28 | G50D, A98S, A426T, W427L |
| NicA2mut214 | 27 | F104I, D130G, G156D, K199R, D295N, T381S |
| NicA2mut216 | 26 | R44H, F104L, V147F, G156C, G317D, H368R, I382V |
| NicA2mut217 | 25 | D130G, S379N |
| NicA2mut219 | 118 | K157R, I161F, M246T, H368R, L449V |
| NicA2mut220 | 117 | V48A, F104L, D130Y, M246T, H368L, E373K, L441M |
| NicA2mut223 | 116 | W171C, D243N, Q366R, S379N, D394N |
| NicA2mut228 | 115 | K314Q, H368RI, N462S |
| NicA2mut229 | 114 | F104I, D130G, G156D, K199R, A209T, F355L, H368Y |
| NicA2mut232 | 113 | F174V, G260S, H368P, W427L |
| NicA2mut233 | 112 | F174V, G260S, W427L |
| NicA2mut234 | 111 | F104L, G317D, D357E, H368R |
| NicA2mut237 | 110 | W108R, D130Y, T183I, G202D, S379N |
| NicA2mut239 | 109 | F104I, D130A |
| NicA2mut240 | 108 | F104L, D130G, E184G, M246T, P278S, I291V |
| NicA2mut2B5 | 100 | A107T, P114Q, G317D, H368R, D389E, N462S |
| NicA2mut2D5 | 99 | K49N, F93L, F104L, V127M, D130S, L132R, F174C, |
| | | T319I, E454D |
| NicA2mut2D9 | 104 | T92A, F174V, E249G, A281T, I298V, F355L, H368R, |
| | | E454D |
| NicA2mut2E3 | 98 | A107T, D130G, T267I, L335V, N462S |
| NicA2mut2E4 | 101 | F104I, G317D, H368R, N462S |
| NicA2mut2F2 | 102 | Q100H, A107T, L112Q, H115Y, H368R |
| NicA2mut2H3 | 103 | S12I, A107T, D130G, T267I, N462S |
| NicA2mut244 | 107 | G50S, F104L, A107T, D130G, G156D, S192C, F235L, |
| | | G239A, A426T, L435Q |
| NicA2mut245 | 106 | F104L, D130G, V231I, L335V, L349M, N462S |
| NicA2mut249 | 105 | A69V, A107T, D130Y, K199R, P278Q, D295N, T381S |
| NicA2mut253 | 119 | F104L, G317D, H368R, L449V, N462S |
| NicA2mut254 | 121 | A107T, D130G, T267I, N462S |
| NicA2mut255 | 122 | F104L, G317D, H368R, L449V, N462S |
| NicA2mut260 | 120 | F104L, G317D, H368R, L449V, N462S, L481P, S482E, |
| | | 483L, 484I |
| NicA2mut302 | 123 | A107T, L112M, D357E, H368R, G406D, L449V, N462S |
| NicA2mut303 | 124 | Y54F, V59I, F63L, A107T, V127M, D130S, L132R, F235I, |
| | | T280K, G317D |
| NicA2mut304 | 125 | V48A, F52L, F104L, W171R, E249G, H368R, T381S, |
| | | N462S |
| NicA2mut305 | 126 | F93L, F104L, V127M, D130S, L132R, S213T, T222S, |
| | | A281T, L374M, M403I |
| NicA2mut306 | 127 | F93L, F104L, V127M, D130S, L132R, I198F, K331N |
| NicA2mut307 | 128 | F104L, A107T, S146I, G317D, H368R, L449V, N462S |
| NicA2mut313 | 129 | F104L, E129V, S192N, E249G, G317D, H368R, T381S, |
| | | N462S |
| NicA2mut314 | 132 | G51S, G73S, R96S, F104L, A107T, L112M, E249G, |
| | | S276C, H368R, G411S, N462S |
| NicA2mut315 | 133 | A107T, L112M, F235Y, G260S, A339V, Q359R, H368R, |
| | | N462S |
| NicA2mut320 | 134 | F104L, A107T, D130S, T319I, L449V, N462S, L480P, |
| | | L481A, S482E, 483L, 484I |
| NicA2mut321 | 135 | F104L, A107T, S146I, K191N, Q210H, G317D, H368R, |
| | | L449V, N462S |
| NicA2mut323 | 136 | F104L, A107T, D130S, M215L, E221D, T319I, L449V, |
| | | N462S |
| NicA2mut324 | 137 | F104L, D130N, H368R, N462S |
| NicA2mut325 | 138 | D130G, D296E, G317S, Q359E, S371R, S379N, T418S, |
| | | L449V |
| NicA2mut326 | 139 | F104L, D130S, H368R, N462S |
| NicA2mut329 | 140 | F104L, A107T, D130S, T293S, T307P, T319I, L449V, |
| | | N462S |

¹D243N: Aspartic acid substitution for Asparagine at position 243 of NicA2.

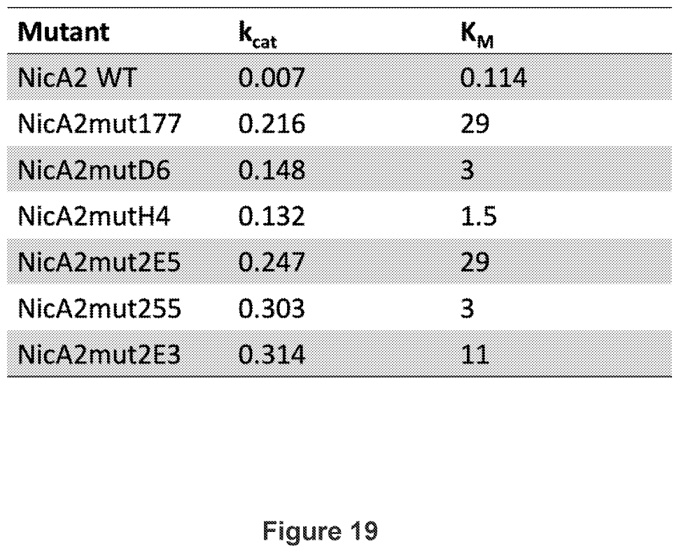

Isolated colonies from the above selection contained variants of NicA2 with improved O₂-dependent nicotine-degrading activity (FIG. 19). These variants were sequenced and the sequences were aligned, revealing that some amino acid positions (for example, D130 of SEQ ID NO:131) were highly substituted in the positive selection hits. This indicates that it is a residue important for modulating oxygen reactivity in the enzyme. We designated this position, and others similarly identified, as “hot spots” where either single amino acid substitutions, or combinations thereof, resulted in large increases in activity. The location of mutational “hot spots” where activity with O₂ was modulated, included F93, F104, A107, W108, D130, L132, E249, G317, H368, S379, T381, W427, and N462 of SEQ ID NO:131. Substitutions at these amino acid locations, or combinations thereof, are expected to exhibit higher activity using oxygen as an electron acceptor.

The resulting nicA2 polynucleotide variants contained the wild-type nicA2 polynucleotide sequence of SEQ ID NO:130, with single nucleotide substitutions relative to that wild-type sequence at the position(s) indicated in Table 3.

TABLE 3

| Polynucleotide Variant | Variant Nucleotides |
|---|---|
| nica2mut1 | G727A, A940C, A1103G |
| nicA2mut5 | G1280T, C1328T |
| nicA2mut6 | G184A, G225T, C232G, A274T, A1103G |
| nicA2mut7 | A389T, G439C, G466A, C800G, G1232A |
| nicA2mut8 | C286A, A389G |
| nicA2mut9 | A389T, A481G, A564T |
| nicA2mut10 | G149A, C275T, T1439C, G1440T, C1441G, |
| | T1442C, G1443T, A1444G, G1445A, |
| | C1446G, T1447C, A1448T, A1449A, 1450A, |
| | 1451T, 1452C |
| nicA2mut11 | G319A, C530T, A874C |
| nicA2mut12 | C343T, A772G |
| nicA2mut17 | G85A, T310A, C668A, G795A |
| nicA2mut19 | T214A, T650C, G922T |
| nicA2mut20 | C253T, C341A, G1232A |
| nicA2mut21 | G110A, A1076G, A1103G |
| nicA2mut22 | A389G, G1292A, C1382T |
| nicA2mut23 | A280G, C391T, G841A, G905C |
| nicA2mut25 | T390G, T672A, G1052A, A1291A |
| nicA2mut31 | G225T, C1286T |
| nicA2mut35 | C413T, T520A, A884T, C1102T, A1363G |
| nicA2mut36 | A115T |
| nicA2mut40 | C398T, C403A, G1420A |
| nicA2mut43 | G1092T, A1326T |
| nicA2mut45 | G225T |
| nicA2mut61 | C757A, A1010T |
| nicA2mut64 | A515G, G1012A, G1168A, A1379T |
| nicA2mut65 | A136C, G319C, G829A, A892T, A1427T |
| nicA2mut66 | C1142T, C1331G |
| nicA2mut75 | G388A |
| nicA2mut95 | T187G, G287A, G388A, G829A, G1136A, |
| | T1439C, G1440T, C1441G, T1442C, |
| | G1443T, A1444G, G1445A, C1446G, |
| | T1447C, A1448T, A1449A, 1450A, 1451T, |
| | 1452C |
| nicA2mut96 | A1103G, T1304A |
| nicA2mutD1 | G434C, G575T, A1076T, A1097G, G1136A |
| nicA2mutH3 | C286A, C312A, G360T, A1103G |
| nicA2mutH4 | C391T, A673C, C1382T |
| nicA2mut101 | G133A, C1096A, A1138G |
| nicA2mut105 | G385C, G388A, G452T, G479A |
| nicA2mut106 | G860A, G1052A |
| nicA2mut112 | C293A, A389C |
| nicA2mut113 | G309C, G550A |
| nicA2mut117 | C539T, C731T, A772G, A1385G |
| nicA2mut118 | A410G, T650C, A1370C |
| nicA2mut119 | G827A, A876C, A970G, G1052A |
| nicA2mut123 | G225T, C391T, C454T, A475G |
| nicA2mut130 | C391T, G829A, G844A |
| nicA2mut136 | G148T, C1177T |
| nicA2mut137 | T143C, T310C, G388T, C800T, T1304A |
| nicA2mut138 | A436G, A1103G, G1294A |
| nicA2mut144 | A389G, C392A, G575A, A916G, C1418T |
| nicA2mut145 | A389G, A470T, T518C, A572T |
| nicA2mut146 | G254A, C686T, G1052A, G1310A |
| nicA2mut147 | G466T, C800G, G1280T |
| nicA2mut149 | A1103G |
| nicA2mut152 | C800T, G1280T |
| nicA2mut155 | A389G, G1136A |
| nicA2mut158 | T322C, G388T, C548T, G1209C |
| nicA2mut160 | A115T, A470G, A481T, T737C, A1103C, |
| | G1117A |
| nicA2mut163 | C403A, A1103G, C1345G |
| nicA2mut167 | A389G, G586C, T737C, A993T |
| nicA2mut169 | G149C, T650C, G779C, G1033T |
| nicA2mut173 | G388A, T908G, A932G, C1184T |
| nicA2mut174 | A124G, G233A, A389T, G675C, T734A |
| nicA2mut175 | T310C, G950A, A1103G |
| nicA2mut177 | G434C, G575T, A1076T, A1097G, G1136A |
| nicA2mut179 | T310C, A389G, G808T, C1177T |
| nicA2mut180 | G467A, C504A, A875G, A970G, G1052A |
| nicA2mut183 | T322C, G388T, A1010T, G1193T |
| nicA2mut189 | G997A, G1052A, T1271A |
| nicA2mut191 | T310A, A389G, G467A, A596G, G883A, |
| | C1142G |
| nicA2mut192 | G296A, A389T, A1362T |
| nicA2mut194 | T520G, G778A, G1280T |
| nicA2mut198 | A746G, A1103G |
| nicA2mut201 | A389G, G560T, G691A, T724A, G1224T |
| nicA2mut202 | C134T, A389T, A1162T |
| nicA2mut204 | G319A, A389T, T989C |
| nicA2mut208 | G1232A, G1280T |
| nicA2mut210 | G149A, G292T, G1276A, G1280T |
| nicA2mut214 | T310A, A389G, G467A, A596G, G883A, |
| | A1141T |
| nicA2mut216 | G131A, T310C, G439T, G466T, G950A, |
| | A1103G, A1144G |
| nicA2mut217 | A389G, G1136A |
| nicA2mut219 | A470G, A481T, T737C, A1103G, C1345G |
| nicA2mut220 | T143C, T310C, G388T, T737C, A1103C, |
| | G1117A, C1321A |
| nicA2mut223 | G513C, G727A, A1097G, G1136A, G1180A |
| nicA2mut228 | A940C, A1103G, A1385G |
| nicA2mut229 | T310A, A389G, G467A, A596G, G625A, |
| | T1065A, C1102T |
| nicA2mut232 | T520G, G778A, A1103C, G1280T |
| nicA2mut233 | T520G, G778A, G1280T |
| nicA2mut234 | T310C, G950A, T1071A, A1103G |
| nicA2mut237 | T322C, G388T, C548T, G605A, G1136A |
| nicA2mut239 | T310A, A389C |
| nicA2mut240 | C312A, A389G, A551G, T737C, C832T, |
| | A871G |
| nicA2mut2B5 | G319A, C341A, G950A, A1103G, C1167A, |
| | A1385G |
| nicA2mut2D5 | A147C, T277C, T310C, G379A, G388T, |
| | A389C, T395G, T521G, C956T, A1362T |
| nicA2mut2D9 | A274G, T520G, A746G, G841A, A892G, |
| | T1065A, A1103G, A1362T |
| nicA2mut2E3 | G319A, A389G, C800T, C1003G, A1385G, |
| | C1386T |
| nicA2mut2E4 | T310A, G950A, A1103G, A1385G |
| nicA2mut2F2 | A300T, G319A, T335A, C343T, A1103G |
| nicA2mut2H3 | G35A, G319A, A389G, C800T, A1385G, |
| | C1386T |
| nicA2mut244 | G148A, T310C, G319A, A389G, G467A, |
| | A574T, T705A, G716C, G1276A, T1304A |
| nicA2mut245 | T310C, A389G, G691A, C1003G, C1045A, |
| | A1385G |
| nicA2mut249 | C206T, G319A, G388T, A596G, C833A, |
| | G883A, C1142G |
| nicA2mut253 | C312A, G950A, A1103G, C1345G, A1385G |
| nicA2mut254 | G319A, A389G, C800T, A1385G |
| nicA2mut255 | C312A, G950A, A1103G, C1345G, A1385G, |
| | C1386T |
| nicA2mut260 | T310C, G950A, A1103G, C1345G, A1385G, |
| | T1442C, G1443T, A1444G, G1445A, |
| | C1446G, T1447C, A1448T, A1449A, 1450A, |
| | 1451T, 1452C |
| nicA2mut302 | G319A, C334A, T1071A, A1103G, G1217A, |
| | C1345G, A1385G |
| nicA2mut303 | A161T, G175A, T187C, G319A, G379A, |
| | G388T, A389C, T395G, T703A, C839A, |
| | C840A, G950A, G1224T, C1262T |
| nicA2mut304 | T143C, C156A, T310C, T511C, A746G, |
| | A1103G, C1142G, A1385G |
| nicA2mut305 | T277C, T310C, G379A, G388T, A389C, |
| | T395G, G638C, A664T, G841A, C1120A, |
| | G1209C |
| nicA2mut306 | T277C, C312A, G379A, G388T, A389C, |
| | T395G, A592T, A993T |
| nicA2mut307 | T310C, G319A, G437T, G950A, A1103G, |
| | C1345G, A1385G, C1386T |
| nicA2mut313 | T310C, A386T, G575A, A746G, G950A, |
| | A1103G, A1141T, A1385G, C1386T |
| nicA2mut314 | G151A, G217A, C286A, C312A, G319A, |
| | C334A, A746G, A826T, A1103G, G1231A, |
| | A1385G |
| nicA2mut315 | G319A, C334A, F235Y, G778A, A339V, |
| | A1076G, A1103G, A1385G |
| nicA2mut320 | T310C, G319A, G388T, A389C, C956T, |
| | C1345G, A1385G, C1386T, T1439C, |
| | G1440T, C1441G, T1442C, G1443T, |
| | A1444G, G1445A, C1446G, T1447C, |
| | A1448T, A1449A, 1450A, 1451T, 1452C |
| nicA2mut321 | C312A, G319A, G437T, G573T, G630T, |
| | G950A, A1103G, C1345G, A1385G, C1386T |
| nicA2mut323 | C312A, G319A, G388T, A389C, A643T, |
| | G663T, C956T, C1345G, A1385G, C1386T |
| nicA2mut324 | C312A, G388A, A1103G, A1385G |
| nicA2mut325 | A389G, C888G, G949A, C1075G, C1113G, |
| | G1136A, A1252T, C1254A, C1345G |
| nicA2mut326 | T310C, G388T, A389C, A1103G, A1385G |
| nicA2mut329 | C312A, G319A, G388T, A389C, A877T, |
| | A919C, C956T, C1345G, A1385G, C1386T |

As disclosed above, the poor O₂ reactivity of NicA2 was addressed using directed evolution to engineer NicA2 to react better with O₂. This strategy has generated a more potent O₂-dependent nicotine-degrading enzyme useful in tobacco-cessation therapies. For NicA2, the selective pressure of nicotine toxicity on E. coli, or use of a genetically modified P. putida S16 ΔnicA2 ΔcycN strain, was exploited to objectively interrogate the importance of all components of the enzyme on its ability to react with O₂. These selections have allowed us to probe the importance of individual residues involved in substrate binding on the reactivity with O₂, as nicotine binding and O₂ reactivity may be inversely linked. This approach has contributed to our understanding of how natural selection has suppressed the reactivity of NicA2's FAD with O₂, even though it belongs to the monoamine oxidase structural family of enzymes that usually react rapidly with O₂.

Rather than relying on potentially imperfect guidance provided by comparing structures, every component of NicA2's 445-amino-acid sequence (mature size) was evaluated for its impact on suppressing the reactivity of NicA2's FAD with O₂. This required a high-throughput assay that was used to rapidly screen through NicA2 mutants to identify ones that react rapidly with O₂. To avoid enhancing the reactivity with O₂ at the expense of reacting with nicotine, the inherent toxicity of nicotine towards E. coli was used as a selective readout in order to screen through large numbers of NicA2 variants for the ability to react rapidly with both O₂ and nicotine. Notably, E. coli's aerobic respiratory chain lacks a diffusible cytochrome c, instead using quinones to link the flow of electrons from dehydrogenases to terminal oxidases. This fact reduced the possibility that NicA2 variants were identified in the selection that coupled nicotine oxidation to the electron transport chain instead of using O₂ as the electron acceptor. The GeneMorph II Random Mutagenesis Kit (Agilent®) and passage through the XL-1 Red E. coli mutator strain (Agilent®) were used as parallel, independent strategies for generating a library of NicA2 mutants in a pBAD expression vector (Copp et al., 2014; Guzman et al., 1995). Both methods of generating random mutant libraries are embodied in commercially available kits. The library of NicA2 expression constructs was then transformed into E. coli and selected for growth on concentrations of nicotine under aerobic conditions where the wild-type enzyme was unable to detoxify the nicotine and allow for growth. Plasmids from colonies that survived were isolated and retransformed into a clean strain background to verify that the enhanced nicotine tolerance was specifically due to the NicA2 variant and not to mutations in the host background. After subsequently identifying the performance-enhancing mutation(s), they were run through additional rounds of mutagenesis and selection on even higher nicotine concentrations until the most potent O₂-dependent nicotine-consuming NicA2 variant(s) was reached. Variants that conferred improved nicotine resistance on E. coli were purified and characterized by stopped-flow analyses to quantitatively determine the improvement in their performance. The resulting variants, and the nature of the amino acid variations, are presented in Table 2 and can be confirmed by inspection of the sequences identified in Table 2 and in the sequence listing.

Additionally, if CycN is, as expected, the physiological electron acceptor, then a ΔnicA2ΔcycN double deletion or ΔcycN single deletion strain of P. putida S16 was available for use as the host organism in a selection to identify NicA2 variants with improved O₂-dependent activity. ΔcycN P. putida S16 expressing wild-type NicA2 from a plasmid is unable to grow, or grows poorly because nicotine turnover should only be possible by using O₂ as the electron acceptor in this strain when cultured on media containing nicotine as the sole carbon and nitrogen source. NicA2 variants expressed from the plasmid-based library with an enhanced ability to use O₂ are identifiable as displaying faster growth and a correspondingly larger colony size on nicotine media plates. Another experimental strategy takes advantage of a recently developed GFP-based nicotine biosensor⁵⁸. This biosensor specifically recognizes nicotine, displaying low fluorescence in the absence of nicotine and high GFP fluorescence when bound to nicotine. The library of NicA2 variants in E. coli is co-expressed with this nicotine biosensor in the presence of nicotine and then fluorescence-activated cell sorting is used to rapidly screen through the library of NicA2 expressers to identify cells with lower GFP-fluorescence, corresponding to higher nicotine oxidase activity. Yet another experimental strategy makes use of the increase in UV absorbance that accompanies the conversion of nicotine to pseudooxynicotine. This spectrophotometric readout is used, e.g., in 96-well plate assays. To accommodate the relatively low throughput of this assay, mutagenesis libraries may be focused on targeted NicA2 residues in the active site and those that line solvent-access channels (identified using CAVER; Jurcik et al., 2018).

The experiments are expected to identify the structural features of NicA2 that are responsible for restricting its FAD's ability to react with O₂. The performance-enhanced enzyme variants overcome the limitations of previously developed enzyme-based tobacco-cessation therapies—namely, the excessively high doses of enzyme needed due to the low O₂-dependent nicotine oxidase activity of the wild-type enzymes. The enzyme variants disclosed herein are enzyme-based tobacco-cessation therapeutics useful in methods of treating nicotine dependence or use.

### REFERENCES

1. Massey V. The Chemical and Biological Versatility of Riboflavin. Biochem Soc Trans. 2000; 28(4):283. doi:10.1042/0300-5127:0280283
2. EI-Gebali S, Mistry J, Bateman A, et al. The Pfam protein families database in 2019. Nucleic Acids Res. 2019; 47(D1):D427-D432. doi:10.1093/nar/gky995
3. Fitzpatrick P F. Oxidation of amines by flavoproteins. Arch Biochem Biophys. 2010; 493(1):13-25. doi:10.1016/j.abb.2009.07.019
4. Margarita A. Tararina K N A. Bioinformatic Analysis of the Flavin-Dependent Amine Oxidase Superfamily: Adaptations for Substrate Specificity and Catalytic Diversity. Neurosci Biobehav Rev. 2020:100632. doi:10.1016/j.neubiorev.2019.07.019
5. Binda C, Mattevi A, Edmondson D E. Structure-function relationships in flavoenzyme-dependent amine oxidations: A comparison of polyamine oxidase and monoamine oxidase. J Biol Chem. 2002; 277(27):23973-23976. doi:10.1074/jbc.R200005200
6. Tang H, Wang L, Meng X, et al. Novel nicotine oxidoreductase-encoding gene involved in nicotine degradation by Pseudomonas putida strain S16. Appl Environ Microbiol. 2009; 75(3):772-778. doi:10.1128/AEM.02300-08
7. Tang H, Wang L, Wang W, et al. Systematic Unraveling of the Unsolved Pathway of Nicotine Degradation in Pseudomonas. PLoS Genet. 2013; 9(10). doi:10.1371/journal.pgen.1003923
8. Fitzpatrick P F. The enzymes of microbial nicotine metabolism. Beilstein J Org Chem. 2018; 14:2295-2307. doi:10.3762/bjoc.14.204
9. Tararina M A, Xue S, Smith L C, et al. Crystallography Coupled with Kinetic Analysis Provides Mechanistic Underpinnings of a Nicotine-Degrading Enzyme. Biochemistry. 2018; 57(26):3741-3751. doi:10.1021/acs.biochem.8b00384
10. Xue S, Schlosburg J E, Janda K D. A new strategy for smoking cessation: Characterization of a bacterial enzyme for the degradation of nicotine. J Am Chem Soc. 2015; 137(32):10136-10139. doi:10.1021/jacs.5b06605
11. Pentel P R, Raleigh M D, LeSage M G, et al. The nicotine-degrading enzyme NicA2 reduces nicotine levels in blood, nicotine distribution to brain, and nicotine discrimination and reinforcement in rats. BMC Biotechnol. 2018; 18(1):1-14. doi:10.1186/s12896-018-0457-7
12. Thisted T, Biesova Z, Walmacq C, et al. Optimization of a nicotine degrading enzyme for potential use in treatment of nicotine addiction. BMC Biotechnol. 2019; 19(1):1-16. doi:10.1186/s12896-019-0551-5
13. Kallupi M, Xue S, Zhou B, Janda K D, George O. An enzymatic approach reverses nicotine dependence, decreases compulsive-like intake, and prevents relapse. Sci Adv. 2018; 4(10):eaat4751. doi:10.1126/sciadv.aat4751
14. Benowitz N L. Nicotine addiction. N Engl J Med. 2010; 362(24):2295. doi:10.1056/NEJMra0809890
15. Tararina M A, Janda K D, Allen K N. Structural Analysis Provides Mechanistic Insight into Nicotine Oxidoreductase from Pseudomonas putida. Biochemistry. 2016; 55(48):6595-6598. doi:10.1021/acs.biochem.6b00963
16. Yu H, Tang H, Wang L, Yao Y, Wu G, Xu P. Complete genome sequence of the nicotine-degrading Pseudomonas putida strain S16. J Bacteriol. 2011; 193(19):5541-5542. doi:10.1128/JB.05663-11
17. Almagro Armenteros J J, Tsirigos K D, Sonderby C K, et al. SignalP 5.0 improves signal peptide predictions using deep neural networks. Nat Biotechnol. 2019; 37(4):420-423. doi:10.1038/541587-019-0036-z
18. Hmelo L R, Borlee B R, Almblad H, et al. Precision-engineering the Pseudomonas aeruginosa genome with two-step allelic exchange. Nat Protoc. 2015; 10(11):1820-1841. doi:10.1038/nprot.2015.115
19. Leferink N G H, Van Den Berg W A M, Van Berkel W J H. L-Galactono-γ-lactone dehydrogenase from Arabidopsis thaliana, a flavoprotein involved in vitamin C biosynthesis. FEBS J. 2008; 275(4):713-726. doi:10.1111/j.1742-4658.2007.06233.x
20. Kuwahara T, White R A, Agosin M. A cytosolic flavin-containing enzyme catalyzing reduction of cytochrome c in Trypanosoma cruzi: Kinetic studies with cytochrome c as substrate. Arch Biochem Biophys. 1985; 241(1):45-49. doi:10.1016/0003-9861(85)90359-5
21. Butt W D, KeilinN D. Absorption spectra and some other properties of cytochrome c and of its compounds with ligands. Proc R Soc Lond B Biol Sci. 1962; 156(November):429-458. doi:10.1098/rspb.1962.0049
22. Henderson Pozzi M, Fitzpatrick P F. A lysine conserved in the monoamine oxidase family is involved in oxidation of the reduced flavin in mouse polyamine oxidase. Arch Biochem Biophys. 2010; 498(2):83-88. doi:10.1016/j.abb.2010.04.015
23. Ugidos A, Morales G, Rial E, Williams H D, Rojo F. The coordinate regulation of multiple terminal oxidases by the Pseudomonas putida ANR global regulator. Environ Microbiol. 2008; 10(7):1690-1702. doi:10.1111/j.1462-2920.2008.01586.x
24. Arai H. Regulation and function of versatile aerobic and anaerobic respiratory metabolism in Pseudomonas aeruginosa. Front Microbiol. 2011; 2(MAY):1-13. doi:10.3389/fmicb.2011.00103
25. McDonald C A, Fagan R L, Collard F, Monnier V M, Palfey B A. Oxygen reactivity in flavoenzymes: Context matters. J Am Chem Soc. 2011; 133(42):16809-16811. doi:10.1021/ja2081873
26. Wang J, Edmondson D E. Topological probes of monoamine oxidases A and B in rat liver mitochondria: Inhibition by TEMPO-substituted pargyline analogues and inactivation by proteolysis. Biochemistry. 2011; 50(13):2499-2505. doi:10.1021/bi101722b
27. Zhuang Z, Marks B, McCauley R B. The insertion of monoamine oxidase A into the outer membrane of rat liver mitochondria. J Biol Chem. 1992; 267(1):591-596.
28. Russell S M, Davey J, Mayer R J. The vectorial orientation of human monoamine oxidase in the mitochondrial outer membrane. Biochem J. 1979; 181(1):7-14. doi:10.1042/bj1810007
29. Gaweska H, Fitzpatrick P F. Structures and Mechanism of the Monoamine Oxidase Family. 2012; 2(5):365-377. doi:10.1515/BMC.2011.030.Structures
30. Graves S M, Xie Z, Stout K A, et al. Dopamine metabolism by a monoamine oxidase mitochondrial shuttle activates the electron transport chain. Nat Neurosci. 2020; 23(1):15-20. doi:10.1038/s41593-019-0556-3
31. Howe G K, Clapp R W. Are we winning or losing the war on cancer? Deciphering the propaganda of NCI's 33-year war. New Solut. 2004; 14(2):109-24. PMID: 17208743.
32. U.S. Department of Health and Human Services. The Health Consequences of Smoking: 50 Years of Progress. A Report of the Surgeon General. Atlanta, Ga.: U.S. Department of Health and Human Services, Centers for Disease Control and Prevention, National Center for Chronic Disease Prevention and Health Promotion, Office on Smoking and Health, 2014.
33. Benowitz N L. Nicotine addiction. N Engl J Med. 2010 Jun. 17; 362(24):2295-303. doi: 10.1056/NEJMra0809890. Review. PMID: 20554984
34. Birge M, Duffy S, Miler J A, Hajek P. What Proportion of People Who Try One Cigarette Become Daily Smokers? A Meta-Analysis of Representative Surveys. Nicotine Tob Res. 2018 Nov. 15; 20(12):1427-1433. doi: 10.1093/ntr/ntx243. PMID: 29126298.
35. Kallupi M, Xue S, Zhou B, Janda K D, George O. An enzymatic approach reverses nicotine dependence, decreases compulsive-like intake, and prevents relapse. Sci Adv. 2018 Oct. 17; 4(10):eaat4751. doi: 10.1126/sciadv.aat4751. eCollection 2018 October PMID: 30345354.
36. Yu H, Tang H, Wang L, Yao Y, Wu G, Xu P. Complete genome sequence of the nicotine-degrading Pseudomonas putida strain S16. J Bacteriol. 2011 October; 193(19):5541-2. doi: 10.1128/JB.05663-11. PMID: 21914868.
37. Xue S, Schlosburg J E, Janda K D. A New Strategy for Smoking Cessation: Characterization of a Bacterial Enzyme for the Degradation of Nicotine. J Am Chem Soc. 2015 Aug. 19; 137(32):10136-9. doi: 10.1021/jacs.5b06605. Epub 2015 Aug. 6. PMID: 26237398.
38. Fitzpatrick P F, Chadegani F, Zhang S, Roberts K M, Hinck C S. Mechanism of the Flavoprotein L-Hydroxynicotine Oxidase: Kinetic Mechanism, Substrate Specificity, Reaction Product, and Roles of Active-Site Residues. Biochemistry. 2016 Feb. 2; 55(4):697-703. doi: 10.1021/acs.biochem.5b01325. Epub 2016 Jan. 15. PMID: 26744768.
39. Wang S N, Xu P, Tang H Z, Meng J, Liu X L, Huang J, Chen H, Du Y, Blankespoor H D. Biodegradation and detoxification of nicotine in tobacco solid waste by a Pseudomonas sp. Biotechnol Lett. 2004 October; 26(19):1493-6. PMID: 15604785.
40. Fagan, R. L. & Palfey, B. A. in Comprehensive Natural Products Chemistry II, Vol 7: Cofactor Biosynthesis and Enzymology (ed. Begley, T. P.) 37-113 (Elsevier Ltd., 2010).
41. Bader M, Muse W, Ballou D P, Gassner C, Bardwell J C. Oxidative protein folding is driven by the electron transport system. Cell. 1999 Jul. 23; 98(2):217-27. PMID: 10428033.
42. Regeimbal J, Gleiter S, Trumpower B L, Yu C A, Diwakar M, Ballou D P, Bardwell J C. Disulfide bond formation involves a quinhydrone-type charge-transfer complex. Proc Natl Acad Sci USA. 2003 Nov. 25; 100(24):13779-84. Epub 2003 Nov. 11. PMID: 14612576.
43. Tararina M A, Janda K D, Allen K N. Structural Analysis Provides Mechanistic Insight into Nicotine Oxidoreductase from Pseudomonas putida. Biochemistry. 2016 Dec. 6; 55(48):6595-6598. Epub 2016 Nov. 18. PMID: 27933790.
44. Berks B C. The twin-arginine protein translocation pathway. Annu Rev Biochem. 2015; 84:843-64. doi: 10.1146/annurev-biochem-060614-034251. Epub 2014 Dec. 8.

Review. PMID: 25494301.
45. Teufel R, Miyanaga A, Michaudel Q, Stull F, Louie G, Noel J P, Baran P S, Palfey B, Moore B S. Flavin-mediated dual oxidation controls an enzymatic Favorskii-type rearrangement. Nature. 2013 Nov. 28; 503(7477):552-556. doi: 10.1038/nature12643. Epub 2013 Oct. 27. PMID: 24162851.
46. Stull F W, Bernard S M, Sapra A, Smith J L, Zuiderweg E R, Palfey B A. Deprotonations in the Reaction of Flavin-Dependent Thymidylate Synthase. Biochemistry. 2016 Jun. 14; 55(23):3261-9. doi: 10.1021/acs.biochem.6b00510. Epub 2016 Jun. 2. PMID: 27214228.
47. Feng Z, Li W, Ward A, Piggott B J, Larkspur E R, Sternberg P W, Xu X Z. A C. elegans model of nicotine-dependent behavior: regulation by TRP-family channels. Cell. 2006 Nov. 3; 127(3):621-33. PMID: 17081982
48. Rauthan M, Gong J, Liu J, Li Z, Wescott S A, Liu J, Xu X Z S. MicroRNA Regulation of nAChR Expression and Nicotine-Dependent Behavior in C. elegans. Cell Rep. 2017 Nov. 7; 21(6):1434-1441. doi: 10.1016/j.celrep.2017.10.043. PMID: 29117550
49. Mattevi A. To be or not to be an oxidase: challenging the oxygen reactivity of flavoenzymes. Trends Biochem Sci. 2006 May; 31(5):276-83. Epub 2006 Apr. 5. Review. PMID: 16600599.
50. Quan S, Wang L, Petrotchenko E V, Makepeace K A, Horowitz S, Yang J, Zhang Y, Borchers C H, Bardwell J C. Super Spy variants implicate flexibility in chaperone action. Elife. 2014; 3:e01584. doi: 10.7554/eLife.01584. Epub 2014 Feb. 4. PMID: 24497545.
51. Quan S, Koldewey P, Tapley T, Kirsch N, Ruane K M, Pfizenmaier J, Shi R, Hofmann S, Foit L, Ren G, Jakob U, Xu Z, Cygler M, Bardwell J C. Genetic selection designed to stabilize proteins uncovers a chaperone called Spy. Nat Struct Mol Biol. 2011 March; 18(3):262-9. doi: 10.1038/nsmb.2016. Epub 2011 Feb. 13. PMID: 21317898.
52. Pan J L, Sliskovic I, Bardwell J C. Mutants in DsbB that appear to redirect oxidation through the disulfide isomerization pathway. J Mol Biol. 2008 Apr. 11; 377(5):1433-42. doi: 10.1016/j.jmb.2008.01.058. Epub 2008 Jan. 31. PMID: 18325532.
53. Masip L, Pan J L, Haldar S, Penner-Hahn J E, DeLisa M P, Georgiou G, Bardwell J C, Collet J F. An engineered pathway for the formation of protein disulfide bonds. Science. 2004 Feb. 20; 303(5661):1185-9. PMID: 14976313.
54. Bader M W, Hiniker A, Regeimbal J, Goldstone D, Haebel P W, Riemer J, Metcalf P, Bardwell J C. Turning a disulfide isomerase into an oxidase: DsbC mutants that imitate DsbA. EMBO J. 2001 Apr. 2; 20(7):1555-62. PMID: 11285220.
55. Feng Z, Li W, Ward A, Piggott B J, Larkspur E R, Sternberg P W, Xu X Z. A C. elegans model of nicotine-dependent behavior: regulation by TRP-family channels. Cell. 2006 Nov. 3; 127(3):621-33. PMID: 17081982.
56. Londer, Y. Y. Expression of recombinant cytochromes c in E. coli. Methods Mol. Biol. 705, 123-150 (2011).
57. Corpet, F. Multiple sequence alignment with hierarchical clustering. Nucleic Acids Res. 16, 10881-10890 (1988).
58. Shivange et al., Determining the pharmacokinetics of nicotinic drugs in the endoplasmic reticulum using biosensors, J. Gen. Physiol. 151, 738-757 (2019).
59. Laskowski R A, Swindells M B. LigPlot+: multiple ligand-protein interaction diagrams for drug discovery, J. Chem. Inf. Model 51, 2778-2786 (2011).
60. Chaiyen P, Fraaije M W, Mattevi A. The enigmatic reaction of flavins with oxygen. Trends Biochem Sci. 37(9):373-80 (2012)
61. Romero E, Gómez Castellanos J R, Gadda G, Fraaije M W, Mattevi A. Same substrate, many reactions: oxygen activation in flavoenzymes. Chem Rev. 2018; 118(4):1742-1769.
62. Romero P A, Arnold F H. Exploring protein fitness landscapes by directed evolution. Nat Rev Mol Cell Biol. 2009; 10(12):866-76.

Each of the references cited herein is hereby incorporated by reference in its entirety, or in relevant part, as would be apparent from the context of the citation.

The disclosed subject matter has been described with reference to various specific embodiments and techniques. It should be understood, however, that many variations and modifications may be made while remaining within the spirit and scope of the disclosed subject matter.

What is claimed is:

1. A NicA2 nicotine oxidoreductase variant or functional fragment thereof comprising fewer than 10 amino acid substitutions, additions, or deletions from the amino acid sequence set forth in SEQ ID NO:131, wherein the NicA2 nicotine oxidoreductase variant exhibits a higher KM and/or a higher K_{cat} for oxidizing nicotine with oxygen as electron acceptor compared to the wild-type NicA2 nicotine oxidase of SEQ ID NO:131.

2. The NicA2 nicotine oxidoreductase variant of claim 1 or a functional fragment thereof comprising 1 amino acid substitution from the amino acid sequence set forth in SEQ ID NO:131.

3. The NicA2 nicotine oxidoreductase variant of claim 1 wherein the variant comprises an amino acid sequence that varies from the wild-type sequence of SEQ ID NO:131 at one or more of positions 12, 29, 37, 39, 42, 44, 45, 46, 48, 49, 50, 51, 52, 54, 59, 62, 63, 69, 72, 73, 75, 78, 85, 92, 93, 94, 96, 98, 99, 100, 103, 104, 107, 108, 112, 114, 115, 120, 127, 129, 130, 131, 132, 133, 135, 137, 138, 145, 146, 147, 151, 152, 156, 157, 159, 160, 161, 168, 171, 172, 173, 174, 177, 180, 183, 184, 187, 188, 191, 192, 196, 198, 199, 202, 209, 210, 213, 215, 217, 221, 222, 223, 224, 225, 229, 231, 235, 239, 242, 243, 244, 245, 246, 249, 253, 258, 260, 265, 267, 270, 276, 277, 278, 280, 281, 282, 287, 291, 292, 293, 295, 296, 298, 302, 303, 306, 307, 308, 311, 314, 317, 319, 324, 330, 331, 333, 335, 337, 338, 345, 349, 351, 355, 357, 359, 364, 366, 368, 371, 373, 374, 379, 380, 381, 382, 388, 389, 390, 393, 394, 395, 398, 403, 406, 408, 411, 418, 421, 424, 426, 427, 429, 431, 432, 435, 437, 441, 442, 443, 444, 449, 454, 455, 457, 460, 461, 462, 473, 474, 476, 480, 481, 482, 483, and/or 484.

4. The NicA2 nicotine oxidoreductase variant of claim 3 wherein the variant comprises a mature NicA2 nicotine oxidoreductase comprising an amino acid sequence that varies from the wild-type sequence of SEQ ID NO:131 at one or more of positions 42, 44, 45, 46, 48, 49, 50, 51, 52, 54, 59, 62, 63, 69, 72, 73, 75, 78, 85, 92, 93, 94, 96, 98, 99, 100, 103, 104, 107, 108, 112, 114, 115, 120, 127, 129, 130, 131, 132, 133, 135, 137, 138, 145, 146, 147, 151, 152, 156, 157, 159, 160, 161, 168, 171, 172, 173, 174, 177, 180, 183, 184, 187, 188, 191, 192, 196, 198, 199, 202, 209, 210, 213, 215, 217, 221, 222, 223, 224, 225, 229, 231, 235, 239, 242, 243, 244, 245, 246, 249, 253, 258, 260, 265, 267, 270, 276, 277, 278, 280, 281, 282, 287, 291, 292, 293, 295, 296, 298, 302, 303, 306, 307, 308, 311, 314, 317, 319, 324, 330, 331, 333, 335, 337, 338, 345, 349, 351, 355, 357, 359, 364, 366, 368, 371, 373, 374, 379, 380, 381, 382, 388, 389, 390, 393, 394, 395, 398, 403, 406, 408, 411, 418, 421, 424, 426, 427, 429, 431, 432, 435, 437, 441, 442, 443, 444, 449, 454, 455, 457, 460, 461, 462, 473, 474, 476, 480, 481, 482, 483, and/or 484.

5. The NicA2 nicotine oxidoreductase variant of claim 3 wherein the variant comprises an amino acid sequence that varies from the wild-type sequence of SEQ ID NO:131 at one or more of positions 93, 104, 107, 108, 130, 132, 249, 317, 368, 379, 381, 427 or 462.

6. The NicA2 nicotine oxidoreductase variant of claim 4 wherein the variant comprises an amino acid substitution of serine for isoleucine at position 12 (S12I), G29S, S37N, T39S, T42A, R44H, A45T, A45V, S46R, V48A, K49N, G50A, G50C, G50D, G50S, G51S, F52L, Y54F, V59I, G62S, F63L, F63V, A69V, C72S, G73S, Q75H, R78G, R78H, R85C, R85H, T92A, T92I, T92S, F93L, T94A, R96H, R96S, A98E, A98S, G99D, Q100H, E103D, F104I, F104L, A107P, A107T, W108R, L112M, L112Q, P114Q, H115Y, M120I, V127M, E129Q, E129V, D130A, D130E, D130G, D130N, D130S, D130V, D130Y, P131Q, P131S, L132R, T133I, L135M, K137R, T138I, G145A, S146G, S146I, V147F, V147L, S151I, P152S, G156C, G156D, G156S, K157M, K157R, I159V, R160H, I161F, I161V, H168Q, W171C, W171R, E172G, V173A, F174C, F174I, F174V, P177L, P180L, T183I, E184G, E184K, R187L, E188D, K191M, K191N, S192C, S192I, S192N, D196H, I198F, K199R, G202D, A209T, Q210H, S213T, M215L, L217P, E221D, T222S, T223N, D224E, K225N, K225Q, P229L, V231I, F235I, F235L, G239A, Y242N, D243N, A244V, F245Y, M246T, E249G, R253S, T258A, G260A, G260S, M265I, T267I, T267S, G270C, S276C, S276N, V277I, P278Q, P278S, T280K, A281T, V282I, G287D, I291V, K292N, K292Q, K292R, T293S, D295N, D295V, D296E, 1298F, 1298V, G302A, V303A, M306V, T307P, V308L, N311S, K314Q, G317D, G317S, T319I, K324E, 1330T, K331N, G333S, L335V, K337M, G338S, V345L, L349M, R351H, F355L, D357E, Q359L, Q359E, Q359R, W364C, Q366K, Q366R, H368L, H368P, H368R, H368Y, S371R, E373K, L374M, S379N, 1380V, T381I, T381S, 1382V, 1388F, D389E, V390I, R393C, D394N, A395V, R398L, M403I, G406D, E408D, G411D, G411S, T418S, P421L, L424Q, A426T, W427L, A429V, G431D, G431S, V432M, L435Q, R437H, L441M, Q442H, A443V, A444G, L449V, E454D, T455A, N457T, H460L, A461V, N462S, A473V, G474S, E476V, L480P, L481A, L481P, S482E, 483L, and/or 484I.

7. The NicA2 nicotine oxidoreductase variant of claim 2 wherein the variant comprises the sequence set forth in any one of SEQ ID NOs:20-119.

8. The fragment of a NicA2 nicotine oxidoreductase variant of claim 1 wherein the fragment comprises a variant amino acid at a position corresponding to position 93, 104, 107, 108, 130, 132, 249, 317, 368, 379, 381, 427 or 462 of SEQ ID NO:131.

9. The NicA2 nicotine oxidoreductase variant of claim 1 wherein the K_{M} of the variant is greater than 0.114.

10. The NicA2 nicotine oxidoreductase variant of claim 9 wherein the K_{M} of the variant is at least 1.5.

11. The NicA2 nicotine oxidoreductase variant of claim 10 wherein the K_{M} of the variant is 1.5-29.

12. The NicA2 nicotine oxidoreductase variant of claim 1 wherein the K_{cat} of the variant is greater than 0.007.

13. The NicA2 nicotine oxidoreductase variant of claim 12 wherein the K_{cat} of the variant is at least 0.132.

14. NicA2 nicotine oxidoreductase variant of claim 13 wherein the Kcat of the variant is 0.132-0.314.

15. The NicA2 nicotine oxidoreductase variant of claim 1 comprising 1 amino acid deletion from the amino acid sequence set forth in SEQ ID NO:131.

16. The NicA2 nicotine oxidoreductase variant of claim 1 comprising 1 amino acid addition from the amino acid sequence set forth in SEQ ID NO:131.

17. The NicA2 nicotine oxidoreductase variant of claim 1 wherein the amino acid sequence of the variant is at least 90% identical to the amino acid sequence set forth in SEQ ID NO:131.

18. A polynucleotide encoding the NicA2 nicotine oxidoreductase variant of claim 1.

19. The polynucleotide according to claim 18 wherein the encoded NicA2 nicotine oxidoreductase variant comprises at least one nucleotide variant relative to the wild-type nicA2 coding region of SEQ ID NO:130, wherein the at least one nucleotide variant corresponds to position 35, 85, 110, 115, 124, 131, 133, 134, 136, 143, 147, 148, 149, 151, 156, 161, 175, 184, 187, 206, 214, 217, 225, 232, 233, 235, 253, 254, 274, 275, 277, 280, 286, 287, 292, 293, 296, 300, 309, 310, 312, 319, 322, 334, 335, 339, 341, 343, 360, 379, 385, 386, 388, 389, 390, 391, 392, 395, 398, 403, 410, 413, 434, 436, 437, 439, 452, 454, 466, 467, 470, 475, 479, 481, 504, 511, 513, 515, 518, 520, 521, 530, 539, 548, 550, 551, 560, 564, 572, 573, 574, 575, 586, 592, 596, 605, 625, 630, 638, 643, 650, 663, 664, 668, 672, 673, 675, 686, 691, 703, 705, 716, 724, 727, 731, 734, 737, 746, 757, 772, 778, 779, 795, 800, 808, 826, 827, 829, 832, 833, 839, 840, 841, 844, 860, 871, 874, 875, 876, 877, 883, 884, 888, 892, 905, 908, 916, 919, 922, 932, 940, 949, 950, 956, 970, 989, 993, 997, 1003, 1010, 1012, 1033, 1045, 1052, 1065, 1071, 1075, 1076, 1092, 1096, 1097, 1102, 1103, 1113, 1117, 1120, 1136, 1138, 1141, 1142, 1144, 1162, 1167, 1168, 1177, 1180, 1184, 1193, 1209, 1217, 1224, 1231, 1232, 1252, 1254, 1262, 1271, 1276, 1280, 1286, 1291, 1292, 1294, 1304, 1310, 1321, 1326, 1328, 1331, 1345, 1362, 1363, 1370, 1379, 1382, 1385, 1386, 1418, 1420, 1427, 1439, 1440, 1441, 1442, 1443, 1444, 1445, 1446, 1447, 1448, 1449, 1450, 1451, and/or 1452 of SEQ ID NO:130.

20. The polynucleotide of claim 18 wherein the variant nucleotide corresponds to position 310, 312, 319, 388, 389, 950, 1052, 1103, 1136, 1280, 1345, 1385, and/or 1386 of SEQ ID NO:130.

21. The polynucleotide of claim 20 wherein the variant nucleotide corresponds to position 310, 312, 319, 388, 389, 950, 1103, 1345, and/or 1385 of SEQ ID NO:130.

22. The polynucleotide of claim 18 comprising a plurality of nucleotide variations at positions corresponding to positions 1439, 1440, 1441, 1442, 1444, 1445, 1446, 1447, 1449, 1450, 1451 and/or 1452 of SEQ ID NO:130.

23. The polynucleotide of claim 18 encoding the NicA2 nicotine oxidoreductase of any one of claims 2-8.

24. The polynucleotide of claim 18, wherein the polynucleotide comprises the NicA2 variant coding region sequence of nica2mut1, nicA2mut5, nicA2mut6, nicA2mut7, nicA2mut8, nicA2mut9, nicA2mut10, nicA2mut11, nicA2mut12, nicA2mut17, nicA2mut19, nicA2mut20, nicA2mut21, nicA2mut22, nicA2mut23, nicA2mut25, nicA2mut31, nicA2mut35, nicA2mut36, nicA2mut40, nicA2mut43, nicA2mut45, nicA2mut61, nicA2mut64, nicA2mut65, nicA2mut66, nicA2mut75, nicA2mut95, nicA2mut96, nicA2mutD1, nicA2mutH3, nicA2mutH4, nicA2mut101, nicA2mut105, nicA2mut106, nicA2mut112, nicA2mut113, nicA2mut117, nicA2mut118, nicA2mut119, nicA2mut123, nicA2mut130, nicA2mut136, nicA2mut137, nicA2mut138, nicA2mut144, nicA2mut145, nicA2mut146, nicA2mut147, nicA2mut149, nicA2mut152, nicA2mut155, nicA2mut158, nicA2mut160, nicA2mut163, nicA2mut167, nicA2mut169, nicA2mut173, nicA2mut174, nicA2mut175, nicA2mut177, nicA2mut179, nicA2mut180, nicA2mut183, nicA2mut189, nicA2mut191, nicA2mut192, nicA2mut194, nicA2mut198, nicA2mut201, nicA2mut202, nicA2mut204, nicA2mut208, nicA2mut210, nicA2mut214, nicA2mut216, nicA2mut217, nicA2mut219, nicA2mut220, nicA2mut223, nicA2mut228, nicA2mut229, nicA2mut232, nicA2mut233, nicA2mut234, nicA2mut237, nicA2mut239, nicA2mut240, nicA2mut2B5, nicA2mut2D5, nicA2mut2D9, nicA2mut2E3, nicA2mut2E4, nicA2mut2F2, nicA2mut2H3, nicA2mut244, nicA2mut245, nicA2mut249, nicA2mut253, nicA2mut254, nicA2mut255, nicA2mut260, nicA2mut302, nicA2mut303, nicA2mut304, nicA2mut305, nicA2mut306, nicA2mut307, nicA2mut313, nicA2mut314, nicA2mut315, nicA2mut320, nicA2mut321, nicA2mut323, nicA2mut324, nicA2mut325, nicA2mut326, or nicA2mut329.

25. A vector comprising the polynucleotide of claim 18.

26. A host cell comprising the polynucleotide of claim 18.

27. A pharmaceutical formulation comprising the NicA2 nicotine oxidoreductase variant of any one of claims 1-8 and a pharmaceutically acceptable excipient.

28. A pharmaceutical formulation comprising the polynucleotide of claim 18 and a pharmaceutically acceptable excipient.

29. The pharmaceutical formulation of claim 27 further comprising a CycN cytochrome c protein.

30. The pharmaceutical formulation of claim 27 wherein the formulation is contained in an epidermal patch, a liposome, a micelle, an implant, or a nanoparticle.

31. A method of identifying a nicotine oxidoreductase (NicA2) variant using O₂ as an electron acceptor comprising:
(a) culturing a host cell comprising a mutagenized coding region for NicA2 on medium comprising at least 1 mg/mL nicotine; and
(b) identifying the mutagenized coding region for NicA2 in a host cell able to grow on the medium as encoding a nicotine oxidoreductase variant using O₂ as an electron acceptor.

32. The method of claim 31 wherein the coding region for NicA2 is mutagenized prior to introduction into the host cell.

33. The method of claim 31 wherein the coding region for NicA2 is nicA2.

34. The method of claim 31 wherein the mutagenized coding region for NicA2 is subjected to at least one more iteration of the method of claim 32.

35. The method of claim 31 wherein the host cell is Escherichia coli or Pseudomonas putida S16.

36. The method of claim 35 wherein the host cell is Pseudomonas putida S16 comprising the partial genotype of ΔnicA2 ΔcycN or ΔcycN alone.

37. The method of claim 31 wherein the host cell comprises a coding region for either an iNicSnFR3a nicotine biosensor or an iNicSnFR3b nicotine biosensor, further wherein the host cell culture is subjected to fluorescence-activated cell sorting, further wherein the host cell comprising the coding region for the NicA2 variant using O₂ as electron acceptor is identified if the fluorescence level is lower than a control.

38. The method of claim 37 wherein the control is a host cell comprising a coding region for wild-type NicA2 and a coding region for either an iNicSnFR3a nicotine biosensor or an iNicSnFR3b nicotine biosensor.

39. A method of reducing nicotine dependence in a subject comprising administering a therapeutically effective amount of
(a) a composition comprising a NicA2 nicotine oxidoreductase variant comprising fewer than 10 amino acid substitutions, additions, or deletions from the amino acid sequence set forth in SEQ ID NO:131 and an excipient; or
(b) a composition comprising a nicotine oxidoreductase, a cytochrome c protein, and an excipient.

40. The method of claim 39 wherein the nicotine oxidoreductase is wild-type NicA2 nicotine oxidoreductase.

41. The method of claim 39 wherein the cytochrome c protein is the CycN cytochrome c protein.

42. The method of claim 39 wherein the subject is a current smoker of a tobacco product or a former smoker of a tobacco product at risk of relapse.

43. The method of claim 39 wherein the composition is contained in an epidermal patch, a liposome, a micelle, an implant, or a nanoparticle.

44. A high throughput screen for a NicA2 nicotine oxidoreductase variant using O₂ as electron acceptor comprising: (a) contacting a plurality of NicA2 nicotine oxidoreductase mutants with nicotine, 10-acetyl-3,7-dihydroxyphenoxazine, and horseradish peroxidase; (b) measuring the production of H₂O₂ by each variant; and (c) identifying a NicA2 nicotine oxidoreductase variant as using O₂ as electron acceptor if the level of H₂O₂ produced is greater than the H₂O₂ produced by a control.

45. The method of claim 44 wherein the control is the wild-type NicA2 nicotine oxidase of SEQ ID NO:131.


SEQUENCE LISTING (140 sequences)

SEQ ID NO: 1 - DNA, 24 bp, Artificial Sequence
  Synthetic Polynucleotide
tcctctaactgaaacttttgaaag

SEQ ID NO: 2 - DNA, 34 bp, Artificial Sequence
  Synthetic Polynucleotide
atccggggatccgctcttttgacattatcctctg

SEQ ID NO: 3 - DNA, 34 bp, Artificial Sequence
  Synthetic Polynucleotide
atccgggaattcgttcatgttaagcagaatctcg

SEQ ID NO: 4 - DNA, 47 bp, Artificial Sequence
  Synthetic Polynucleotide
ctttcaaaagtttcagttagaggacgcttgtttcattttttatcctc

SEQ ID NO: 5 - DNA, 20 bp, Artificial Sequence
  Synthetic Polynucleotide
gattcatcaaagaggggcag

SEQ ID NO: 6 - DNA, 20 bp, Artificial Sequence
  Synthetic Polynucleotide
gtattcgttacaggcagagc

SEQ ID NO: 7 - protein, 308 aa, Artificial Sequence
  Synthetic Polypeptide
MTTFWSLYITALTVGTLIALLWLYFATRKGEAKGTTEKTMGHSYDGIEEYDNPLPKWWFMLYIGTIVFSVSYLVLYPGLGNWKGVLPGYEGGWTQDKQWEREMNIAQEKYGPIFAKYAAMPIEEVAKDEHAMKMGSRMFATYCSICHGSDAKGALGFPNLADNEWRWGGDPQSIETTILGGRHAIMAAWGDILGEDGVKNVAAYVRTELAGLKLPEGTKADVEAGKQIFSVNCVACHGPEGKGTALVGAPNLTNPGAFIYGSSYAQLQQTIRHGRQGQMPAQEPYLGKEKVHILAAYIYNLSHNQGSN

SEQ ID NO: 8 - protein, 312 aa, Artificial Sequence
  Synthetic Polypeptide
MTTFWSTYICVLTIGSLIGLTWLLLATRKGQSNNTTDQTMGHSFDGIEEYDNPLPKWWFWLFVGTLVFSVGYLILYPGLGNWKGILPGYENGWTGANEWQKEMEKADARFGPIFAKYAAMPVEEVAKDPQALKMGSRLFASNCSVCHGSDAKGAYGFPNLTDKDWRWGGEAETIKASIMNGRHGVMPAWAEVIGEQGVAVAAFVLTNLDGRSLPEGVKADAAKGKEIFAGNCVACHGPEGKGTPAMGAPDLTHPQAFIYGSSFAQLQQTIRYGRQGQMPAQAEIQGNDKVHLLAAYVYSLSQDQASPDLTAK

SEQ ID NO: 9 - protein, 324 aa, Artificial Sequnce
  Synthetic Polypeptide
MTTFWSLYVTVLTLGTIFSLTWLLLSTRKGQREEVTDETVGHAFDGIEEYDNPLPKWWFWLFVGTIIFALGYLVLYPGLGNWKGVLPGYSYLDNDKQTEFSNGQPGWTGVHEQKEMAKADARFGPIFAKFAAMPITEVAKDPQALKMGARLFASNCSVCHGSDAKGAFGFPNLTDNDWRWGGEPDTIKTTIMGGRHFVMPAWAEVIGDQFVADVAAFVVSKLDGRTLPEGVKADAENGQKIFAANCVACHGPEGKGTPAMGAPNLTHPQAFIYGSSFAQLQQTIRYGRQGQMPAQEQLQGNDKVHLLAAYVYSLSQQAEPAKAE

SEQ ID NO: 10 - protein, 288 aa, Artificial Sequence
  Synthetic Polypeptide
  MISC_FEATURE (9)..(9): Xaa is I or V
  MISC_FEATURE (29)..(29): Xaa is D, E, N, Q or Z
  MISC_FEATURE (32)..(32): Xaa is D, E, N, Q or Z
  MISC_FEATURE (38)..(38): Xaa is F or Y
  MISC_FEATURE (38)..(38): Xaa is F or Y
  MISC_FEATURE (56)..(56): Xaa is F or Y
  MISC_FEATURE (57)..(57): Xaa is I or V
  MISC_FEATURE (61)..(61): Xaa is I or V
  MISC_FEATURE (68)..(68): Xaa is I or V
  MISC_FEATURE (79)..(79): Xaa is I or V
  MISC_FEATURE (84)..(84): Xaa is D, E, N, Q, or Z
  MISC_FEATURE (89)..(89): Xaa is D, E, N, Q, or Z
  MISC_FEATURE (91)..(91): Xaa is D, E, N, Q, or Z
  MISC_FEATURE (97)..(97): Xaa is D, E, N, Q, or Z
  MISC_FEATURE (100)..(100): Xaa is F or Y
  MISC_FEATURE (107)..(107): Xaa is F or Y
  MISC_FEATURE (112)..(112): Xaa is I or V
  MISC_FEATURE (122)..(122): Xaa is L or M
  MISC_FEATURE (128)..(128): Xaa is L or M
  MISC_FEATURE (135)..(135): Xaa is I or V
  MISC_FEATURE (153)..(153): Xaa is D,E, N, Q, or  Z
  MISC_FEATURE (159)..(159): Xaa is D,E, N, Q, or  Z
  MISC_FEATURE (161)..(161): Xaa is D,E, N, Q, or  Z
  MISC_FEATURE (168)..(168): Xaa is L or M
  MISC_FEATURE (174)..(174): Xaa is I or V
  MISC_FEATURE (180)..(180): Xaa is D, E, N, Q, or Z
  MISC_FEATURE (181)..(181): Xaa is I or V
  MISC_FEATURE (184)..(185): Xaa is D, E, N, Q or Z
  MISC_FEATURE (189)..(189): Xaa is D, E, N, Q or Z
  MISC_FEATURE (193)..(193): Xaa is F or Y
  MISC_FEATURE (234)..(234): Xaa is D,E, N, Q, or Z
  MISC_FEATURE (247)..(247): Xaa is F or Y
  MISC_FEATURE (270)..(270): Xaa is D, E, N,Q, Z
  MISC_FEATURE (279)..(279): Xaa is I or V
MTTFWSLYXTVLTGTLILTWLLLATRKGXTTXTMGHSXDGIEEYDNPLPKWWFWLXXGTIXFSVGYLXLYPGLGNWKGXLPGYXGWTGXWXKEMKAXARXGPIFAKXAAMPXEEVAKDPQAXKMGSRXFASNCSXCHGSDAKGAGFPNLTDNXWRWGGXPXTIKTTIXGGRHGXMPAWAXXIGXXGVAXVAAXVTLDGRLPEGVKADAEGKIFANCVACHGPEGKGTPAMGAPXLTHPQAFIYGSSXAQLQQTIRYGRQGQMPAQEQGNXKVHLLAAYXYSLSQQASP

SEQ ID NO: 11 - protein, 490 aa, Pseudomonas putida
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLSLEHHHHHH

SEQ ID NO: 12 - DNA, 1449 bp, Artificial Sequence
  Synthetic Polynucleotide
atgagcgataagaccaaaaccaacgagggtttcagccgtcgtagctttattggcagcgcggcggttgttaccgcgggtgttgcgggtctgggtgcgattgatgcggcgagcgcgacccagaagaccaaccgtgcgagcaccgtgaaaggtggcttcgattacgacgtggttgtggttggtggcggttttgcgggtgcgaccgcggcgcgtgagtgcggtctgcagggttatcgtaccctgctgctggaagcgcgtagccgtctgggcggtcgtaccttcaccagccgttttgcgggtcaagagatcgagttcggcggtgcgtgggttcattggctgcagccgcatgtgtgggcggagatgcaacgttacggcctgggtgtggttgaagatccgctgaccaacctggacaagaccctgatcatgtataacgatggcagcgttgagagcattagcccggacgagttcggcaagaacatccgtattgcgtttgagaaactgtgccacgacgcgtgggaagtgttcccgcgtccgcacgaaccgatgtttaccgagcgtgcgcgtgaactggataagagcagcgtgctggaccgtatcaaaaccctgggtctgagccgtctgcagcaagcgcagattaacagctacatggcgctgtatgcgggcgagaccaccgataagttcggcctgccgggtgttctgaaactgtttgcgtgcggcggttggaactacgatgcgtttatggacaccgaaacccactatcgtatccagggtggcaccatcggcctgattaacgcgatgctgaccgacagcggtgcggaagtgcgtatgagcgttccggtgaccgcggttgaacaagtgaacggcggtgtgaagattaaaaccgacgatgacgaaatcattaccgcgggtgtggttgtgatgaccgtgccgctgaacacctacaaacacatcggcttcaccccggcgctgagcaagggtaaacagcgttttattaaagagggtcaactgagcaagggtgcgaaactgtacgttcacgtgaaacagaacctgggccgtgttttcgcgtttgcggatgaacagcaaccgctgaactgggtgcaaacccacgattatagcgacgagctgggcaccatcctgagcatcaccattgcgcgtaaggaaaccattgacgttaacgatcgtgacgcggttacccgtgaggtgcagaaaatgtttccgggtgttgaggtgctgggcaccgcggcgtatgattggaccgcggacccgtttagcctgggtgcgtgggcggcgtatggtgtgggtcagctgagccgtctgaaggatctgcaagcggcggagggtcgtatcctgtttgcgggtgcggaaaccagcaacggctggcacgcgaacattgatggtgcggttgagagcggtctgcgtgcgggtcgtgaagtgaaacaactgctgagctaa

SEQ ID NO: 13 - protein, 431 aa, Paenarthrobacter nicotinovorans
  MISC_FEATURE (166)..(166): Target of Mutagenesis
  MISC_FEATURE (311)..(311): Target of Mutagenesis
  MISC_FEATURE (326)..(326): Target of Mutagenesis
MYDAIVVGGGFSGLKAARDLTNAGKKVLLLEGGERLGGRAYSRESRNVPGLRVEIGGAYLHRKHHPRLAAELDRYGIPTAAASEFTSFRHRLGPTAVDQAFPIPGSEAVAVEAATYTLLRDAHRIDLEKGLENQDLEDLDIPLNEYVDKLDLPPVSRQFLLAWAWNMLGQPADQASALWMLQLVAAHHYSILGVVLSLDEVFSNGSADLVDAMSQEIPEIRLQTVVTGIDQSGDVVNVTVKDGHAFQAHSVIVATPMNTWRRIVFTPALPERRRSVIEEGHGGQGLKILIHVRGAEAGIECVGDGIFPTLYDYCEVSESERLLVAFTDSGSFDPTDIGAVKDAVLYYLPEVEVLGIDYHDWIADPLFEGPWVAPRVGQFSRVHKELGEPAGRIHFVGSDVSLEFPGYIEGALETAECAVNAILHSHHHHHH

SEQ ID NO: 14 - DNA, 1278 bp, Artificial Sequence
  Synthetic Polynucleotide
atgtatgatgcgattgttgtgggtggcggttttagcggcctgaaagcggcgcgtgacctgaccaatgcgggcaagaaagtgctgctgctggagggtggcgaacgtctgggtggccgtgcgtacagccgtgagagccgtaacgtgccgggtctgcgtgttgaaatcggtggcgcgtacctgcaccgtaagcatcacccgcgtctggcggcggagctggaccgttatggcattccgaccgcggcggcgagcgagttcaccagctttcgtcatcgtctgggtccgaccgcggtggatcaggcgttcccgattccgggtagcgaggcggtggcggttgaagcggcgacctacaccctgctgcgtgatgcgcaccgtatcgatctggagaagggtctggaaaaccaagacctggaggacctggatattccgctgaacgaatatgtggacaaactggatctgccgccggttagccgtcagttcctgctggcgtgggcgtggaacatgctgggtcagccggcggaccaagcgagcgcgctgtggatgctgcaactggttgcggcgcaccactatagcatcctgggtgtggttctgagcctggatgaggtgtttagcaacggcagcgcggacctggttgatgcgatgagccaggagatcccggaaattcgtctgcaaaccgtggttaccggtattgaccagagcggcgatgtggttaacgtgaccgttaaggacggtcacgcgttccaggcgcacagcgtgatcgttgcgaccccgatgaacacctggcgtcgtattgttttcaccccggcgctgccggagcgtcgtcgtagcgtgatcgaggaaggtcacggtggccaaggcctgaaaatcctgattcacgtgcgtggtgcggaggcgggcatcgaatgcgttggtgacggcatttttccgaccctgtacgattattgcgaagtgagcgagagcgaacgtctgctggttgcgttcaccgacagcggtagctttgacccgaccgatatcggcgcggtgaaggatgcggttctgtactacctgccggaagttgaggtgctgggtatcgactaccacgattggattgcggacccgctgtttgagggtccgtgggtggcgccgcgtgttggccaatttagccgtgtgcacaaagagctgggtgaaccggcgggccgtattcacttcgtgggtagcgatgttagcctggaatttccgggctatattgaaggtgcgctggagaccgcggaatgcgcggtgaatgcgatcctgcacagctaa

SEQ ID NO: 15 - DNA, 399 bp, Artificial Sequence
  Synthetic Polynucleotide
atgaaacaagcgctgcaagtggttacccgtcattttggtagcctggttctgctggcgctgagcggtgtggcggttggtgcggataattgcgacccggagaacggtaagaaagtgtaccagatctgcagcgtttgccacagcaacgataccaccggtgtgcacggcgcggcggcgccgaacctgcacggtctggaaggccgtaaagtgggtagcgttccgggcttcaaatttagcagcgcgctgcgtgacagcggtgatacctggaccccgcaacacctggacaagttcctggagaacccgatggcggtttatccgctgacccgtatggcgtttagcggcctgaaaaatgaaaaagaccgtcgtgatgttctgtgcttcctgagcaagagcagcaactaa

SEQ ID NO: 16 - DNA, 1449 bp, Artificial Sequence
  Synthetic Polynucleotide
atgagtgataaaacaaaaacaaatgaaggctttagccgcaggtcttttatcggaagcgcggcagtcgtaacagcaggtgttgcgggattgggagctattgatgcggcttcggctacgcaaaaaacgaaccgagcaagcaccgtcaaaggtggcttcgattacgatgtggtagtagttggtggagggtttgctggcgcgacagccgcccgtgaatgtggtttgcagggttatcgaacgcttttattggaagcgaggtcccgcctaggtggtcgtacgtttacctcgcgctttgcaggtcaagaaattgaatttggcggggcatgggtgcactggctgcagccgcatgtttgggcagaaatgcagcgttacggtctgggtgtagtggaagatccacttactaatttagataaaaccttaatcatgtataacgacggaagcgtcgaaagtatttcgcccgatgaatttggcaaaaacattcgaatagcttttgaaaagctttgtcacgatgcctgggaagtatttcctcgtccgcatgagccgatgtttactgagcgcgctcgggaattggataaatcttctgttcttgatcgcatcaaaactttgggcttaagtcggctgcaacaggctcaaatcaatagttacatggccttgtatgcaggtgagacaactgacaaatttggcctgcctggtgtacttaagttgtttgcatgcggcggttggaactatgacgccttcatggacactgaaactcattatagaattcaagggggcacgataggcctcattaatgcaatgttgaccgatagcggtgccgaggtccgcatgtctgtgcccgtcactgctgttgagcaagtcaatggtggcgtcaaaatcaagaccgacgacgacgaaattattaccgccggagtggtcgtaatgacagttccactcaatacgtataaacatatcggttttacgcctgccctttctaaaggtaaacaacgattcatcaaagaggggcagcttagcaaaggtgctaagctttatgttcatgttaagcagaatctcggacgggtttttgcgtttgcggatgaacagcaacctttaaactgggtccagacgcacgattacagcgacgagttggggacaatactgtcgatcaccatcgctcgcaaagaaacaattgatgtgaatgaccgagatgctgtaactcgcgaagttcaaaaaatgtttccgggtgttgaggttcttggtacagcggcttacgactggacagctgatccattttccttgggggcatgggcggcttatggagtaggtcaactaagtcgtctcaaagatctacaggcggctgaaggacgtattttatttgcaggagctgaaaccagtaacggttggcacgcgaatatcgatggtgctgttgaaagtggactacgtgccggtagggaggttaagcagctcttaagctag

SEQ ID NO: 17 - DNA, 1278 bp, Artificial Sequence
  Synthetic Polynucleotide
ttgtatgacgcgattgtggttggtgggggtttcagcgggctgaaagcagctcgcgatcttaccaacgccggcaagaaggtacttctgcttgaaggcggtgaacgtctgggaggacgggcgtattcccgggagtcccggaatgtacctggcctgcgggtcgagatcggtggagcataccttcaccggaagcaccacccgcgtttggcagctgaattggaccggtatggaattcccactgcagctgcaagcgagttcacctcgtttaggcatcgccttggaccgaccgcagtagaccaggcatttcctattcctggctcagaagcagttgcggtggaagcagccacctacacattgctgcgtgatgcgcacaggatagatctggaaaagggattggaaaaccaggaccttgaggacctggacatcccactgaacgagtatgttgacaaactcgatttgccccctgtttctaggcagttcttgctcgcttgggcatggaacatgcttggccagccggctgaccaagcatccgcgctttggatgctgcaacttgttgcagcgcatcactacagcatcctaggtgttgttctctcgctggatgaagttttttccaatggttccgctgaccttgtcgacgcaatgagccaagaaattcctgaaatcagactgcagactgtcgtgaccggaatagaccagtctggcgatgtagtgaacgtgacggtcaaggatgggcatgccttccaggctcattccgtgattgttgctactccaatgaacacttggcgaaggatcgttttcacccccgcacttcctgaacggcgcaggtcagtaatcgaggaagggcatggtggtcagggtctgaaaattcttatccacgtgcggggagccgaggcaggaattgaatgcgtcggagacggaatatttcccactctctacgattattgcgaggtgtcggaaagcgaacgactgttggtggctttcacggactcaggatccttcgatccgaccgatatcggcgcagtcaaggacgcggtgctttactacttgccggaggttgaagttctgggtatcgactaccacgactggatcgcagacccgcttttcgaagggccctgggtagcaccgcgcgtgggacaatttagtcgagtgcataaggaacttggagagcccgctggccggattcatttcgtaggttccgatgtttcgttagagttccccggatatattgaaggcgcactggaaaccgctgaatgcgcggtgaacgcaattctgcattcgtag

SEQ ID NO: 18 - DNA, 399 bp, Artificial Sequence
  Synthetic Polynucleotide
atgaaacaagcgttgcaagttgttactcgtcattttggttcgttagttcttctagcattatctggtgtagctgtaggtgcagataactgtgatccggaaaatgggaaaaaggtttaccaaatttgcagtgtgtgccactcaaatgacactactggtgttcacggcgcagcggcgcccaatctacacggtctggagggccggaaagtcggctcggtgccgggtttcaaattttcttcagcgctacgtgattctggggacacttggaccccgcagcatttagataagttcctggagaatcctatggccgtatatcccctaacccgtatggctttctcaggcctcaaaaatgaaaaagatcgacgcgacgtgctttgttttttgagcaaatcctctaactga

SEQ ID NO: 19 - protein, 320 aa, Streptomyces sp.
MAPMDPSLVTSVEQSSLTELGEINIDDMIDYVHKDLKKLPEYIDLYKRYFKHRWNAYDLDFSQDAIDWRTKMTEEERDAFKEIAAAFHHAERQVEIELPVFMLSGSEDAKLYLSTQIEDEARHTLFFDRFYREVVGLPGDSVAELLAASFDYVPESFVGSFGLMAHLSDDLRKDPDNQKLRVRYATAYFLWIEGVLALSIMKITLTYCRTRGYLPGYYTGFIASCRDEARHVQFGMRFLRDAIQEDPRLVHEVYETLRTELAMNGGVSKPAPLEPLGWDAEAVTEFMIRQVQNKLSDVGIDLPPDIAAMVQNIQPEIAGG

SEQ ID NO: 20 - protein, 482 aa, Artificial Sequence
  Synthetic
  MISC_FEATURE: NicA2
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 21 - protein, 482 aa, Artificial Sequence
  Synthetic
  MISC_FEATURE: NicA2mut177; G145A, S192I, Q359L, Q366R, S379N
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDASVESISPDEFGKNIRIAFEKLCHDAWEVFPRPHEPMFTERARELDKISVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADELQPLNWVRTHDYSDELGTILNITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 22 - protein, 482 aa, Artificial Sequence
  Synthetic
  MISC_FEATURE: NicA2mutH4
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDSLTNLDKTLIMYNDGSVESISPDEFGKNIRIAFEKLCHDAWEVFPRPHEPMFTERARELDKSSVLDRIKTLGLSRLQQAQINSYMALYAGETTDQFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHVNIDGAVESGLRAGREVKQLLS

SEQ ID NO: 23 - protein, 482 aa, Artificial Sequence; identical to SEQ ID NO: 21
  Synthetic
  MISC_FEATURE: NicA2mutD1

SEQ ID NO: 24 - protein, 482 aa, Artificial Sequence
  Synthetic
  MISC_FEATURE: NicA2mutH3
MSDKTKTNEGFSRRSFIGSAAVVTAGVAGLGAIDAASATQKTNRASTVKGGFDYDVVVVGGGFAGATAARECGLQGYRTLLLEARSRLGGRTFTSSFAGQEIELGGAWVHWLQPHVWAEIQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 25 - protein, 482 aa, Artificial Sequence
  Synthetic
  MISC_FEATURE: NicA2mut217; D130G, S379N
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILNITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 26 - protein, 482 aa, Artificial Sequence
  Synthetic
  MISC_FEATURE: NicA2mut216; R44H, F104L, V147F, G156C, G317D, H368R, I382V
MSDKTKTNEGFSRRSFIGSAAVVTAGVAGLGAIDAASATQKTNHASTVKGGFDYDVVVVGGGFAGATAARECGLQGYRTLLLEARSRLGGRTFTSRFAGQEIELGGAWVHWLQPHVWAEMQRYGLGVVEDPLTNLDKTLIMYNDGSFESISPDEFCKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITVARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 27 - protein, 482 aa, Artificial Sequence
  Synthetic
  MISC_FEATURE: NicA2mut214; F104I, D130G, G156D, K199R, D295N, T381S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEIGGAWVHWLQPHVWAEMQRYGLGVVEGPLTNLDKTLIMYNDGSVESISPDEFDKNIRIAFEKLCHDAWEVFPRPHEPMFTERARELDKSSVLDRIRTLGLSRLQQAQINSYMALYAGETTDKFGLPGVLKLFACGGWNYDAFMDTETHYRIQGGTIGLINAMLTDSGAEVRMSVPVTAVEQVNGGVKIKTDNDEIITAGVVVMTVPLNTYKHIGFTPALSKGKQRFIKEGQLSKGAKLYVHVKQNLGRVFAFADEQQPLNWVQTHDYSDELGTILSISIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 28 - protein, 482 aa, Artificial Sequence
  Synthetic
  MISC_FEATURE: NicA2mut210; G50D, A98S, A426T, W427L
MSDKTKTNEGFSRRSFIGSAAVVTAGVAGLGAIDAASATQKTNRASTVKDGFDYDVVVVGGGFAGATAARECGLQGYRTLLLEARSRLGGRTFTSRFSGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGTLAAYGVGQLSRLKDLQAAEGRILFAGAETSNGWHANIDGAVESGLRAGREVKQLLS

SEQ ID NO: 29 - protein, 482 aa, Artificial Sequence
  Synthetic
  MISC_FEATURE: NicA2mut208; G411D, W427L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLDTAAYDWTADPFSLGALAAYGVGQLSRLKDLQAAEGRILFAGAETSNGWHANIDGAVESGLRAGREVKQLLS

SEQ ID NO: 30 - protein, 482 aa, Artificial Sequence
  Synthetic
  MISC_FEATURE: NicA2mut204; A107T, D130V, I330T
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEVPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFTKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 31 - protein, 482 aa, Artificial Sequence
  Synthetic
  MISC_FEATURE: NicA2mut149; H368R
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 32 - protein, 482 aa, Artificial Sequence
  Synthetic
  MISC_FEATURE: NicA2mut36
MSDKTKTNEGFSRRSFIGSAAVVTAGVAGLGAIDAASASQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 33 - protein, 482 aa, Artificial Sequence
  Synthetic
  MISC_FEATURE: NicA2mut96
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQQSRLKDLQAAEGRILFAGAETSNGWHANIDGAVESGLRAGREVKQLLS

SEQ ID NO: 34 - protein, 482 aa, Artificial Sequence
  Synthetic
  MISC_FEATURE: NicA2mut75
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVENPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 35 - protein, 482 aa, Artificial Sequence
  Synthetic
  MISC_FEATURE: Nica2mut1
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYNAFMDTETHYRIQGGTIGLINAMLTDSGAEVRMSVPVTAVEQVNGGVKIKTDDDEIITAGVVVMTVPLNTYQHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 36 - protein, 482 aa, Artificial Sequence
  Synthetic
  MISC_FEATURE: NicA2mut5
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGALAAYGVGQLSRLKDLQVAEGRILFAGAETSNGWHANIDGAVESGLRAGREVKQLLS

SEQ ID NO: 37 - protein, 482 aa, Artificial Sequence
  Synthetic
  MISC_FEATURE: NicA2mut138; S146G, H368R, V432M
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGGVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGMGQLSRLKDLQAAEGRILFAGAETSNGWHANIDGAVESGLRAGREVKQLLS

SEQ ID NO: 38 - protein, 482 aa, Artificial Sequence; identical to SEQ ID NO: 25
  Synthetic
  MISC_FEATURE: NicA2mut155; D130G, S379N

SEQ ID NO: 39 - protein, 482 aa, Artificial Sequence
  Synthetic
  MISC_FEATURE: NicA2mut175; F104L, G317D, H368R
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 40 - protein, 482 aa, Artificial Sequence
  Synthetic
  MISC_FEATURE: NicA2mut21
MSDKTKTNEGFSRRSFIGSAAVVTAGVAGLGAIDAANATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADERQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 41 - protein, 482 aa, Artificial Sequence
  Synthetic
  MISC_FEATURE: NicA2mut43
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNCVQTHDYSDELGTILSITIARKETIDVNDRDAVTREVQKMFPGVEVLGTAAYDWTADPFSLGAWAAYGVGQLSRLKDLHAAEGRILFAGAETSNGWHANIDGAVESGLRAGREVKQLLS

SEQ ID NO: 42 - protein, 482 aa, Artificial Sequence
  Synthetic
  MISC_FEATURE: NicA2mut106
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNDGVKIKTDDDEIITAGVVVMTVPLNTYKHIGFTPALSKGKQRFIKEGQLSKGAKLYVHVKQNLGHVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 43 - protein, 482 aa, Artificial Sequence
  Synthetic
  MISC_FEATURE: NicA2mut113; E103D, E184K
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIDFGGAWVHWLQPHVWAEMQRYGLGVVEDPLTNLDKTLIMYNDGSVESISPDEFGKNIRIAFEKLCHDAWEVFPRPHEPMFTKRARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 44 - protein, 482 aa, Artificial Sequence
  Synthetic
  MISC_FEATURE: NicA2mut8
MSDKTKTNEGFSRRSFIGSAAVVTAGVAGLGAIDAASATQKTNRASTVKGGFDYDVVVVGGGFAGATAARECGLQGYRTLLLEARSRLGGRTFTSSFAGQEIEFGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 45 - protein, 482 aa, Artificial Sequence
  Synthetic
  MISC_FEATURE: NicA2mut12
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPYVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGAIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 46 - protein, 482 aa, Artificial Sequence
  Synthetic
  MISC_FEATURE: NicA2mut66
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSIIIARKETIDVNDRDAVTREVQKMFPGVEVLGTAAYDWTADPFSLGAWAAYGVGQLSRLKDLQAGEGRILFAGAETSNGWHANIDGAVESGLRAGREVKQLLS

SEQ ID NO: 47 - protein, 482 aa, Artificial Sequence
  Synthetic
  MISC_FEATURE: NicA2mut112
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFEGQEIEFGGAWVHWLQPHVWAEMQRYGLGVVEAPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 48 - protein, 482 aa, Artificial Sequence
  Synthetic
  MISC_FEATURE: NicA2mut61
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYSIQGGTIGLINAMLTDSGAEVRMSVPVTAVEQVNGGVKIKTDDDEIITAGVVVMTVPLNTYKHIGFTPALSKGKQRFIKEGQLSMGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 49 - protein, 482 aa, Artificial Sequence
  Synthetic
  MISC_FEATURE: NicA2mut45
MSDKTKTNEGFSRRSFIGSAAVVTAGVAGLGAIDAASATQKTNRASTVKGGFDYDVVVVGGGFAGATAARECGLHGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 50 - protein, 482 aa, Artificial Sequence
  Synthetic
  MISC_FEATURE: NicA2mut31
MSDKTKTNEGFSRRSFIGSAAVVTAGVAGLGAIDAASATQKTNRASTVKGGFDYDVVVVGGGFAGATAARECGLHGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAVYGVGQLSRLKDLQAAEGRILFAGAETSNGWHANIDGAVESGLRAGREVKQLLS

SEQ ID NO: 51 - protein, 482 aa, Artificial Sequence
  Synthetic
  MISC_FEATURE: NicA2mut11
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEDPLTNLDKTLIMYNDGSVESISPDEFGKNIRIAFEKLCHDAWEVFPRLHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIQTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 52 - protein, 482 aa, Artificial Sequence
  Synthetic
  MISC_FEATURE: NicA2mut19
MSDKTKTNEGFSRRSFIGSAAVVTAGVAGLGAIDAASATQKTNRASTVKGGFDYDVVVVGGGFAGATAARESGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMAPYAGETTDKFGLPGVLKLFACGGWNYDAFMDTETHYRIQGGTIGLINAMLTDSGAEVRMSVPVTAVEQVNGGVKIKTDDDEIITAGVVVMTLPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 53 - protein, 482 aa, Artificial Sequence
  Synthetic
  MISC_FEATURE: NicA2mut20
MSDKTKTNEGFSRRSFIGSAAVVTAGVAGLGAIDAASATQKTNRASTVKGGFDYDVVVVGGGFAGATAARECGLQGYRTLLLEACSRLGGRTFTSRFAGQEIEFGGAWVHWLQQHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLDTAAYDWTADPFSLGAWAAYGVGQLSRLKDLQAAEGRILFAGAETSNGWHANIDGAVESGLRAGREVKQLLS

SEQ ID NO: 54 - protein, 482 aa, Artificial Sequence
  Synthetic
  MISC_FEATURE: NicA2mut136; G50C, R393C
MSDKTKTNEGFSRRSFIGSAAVVTAGVAGLGAIDAASATQKTNRASTVKCGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDCDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 55 - protein, 482 aa, Artificial Sequence
  Synthetic
  MISC_FEATURE: NicA2mut163; L135M, H368R, L449V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNMDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHANIDGAVESGLRAGREVKQLLS

SEQ ID NO: 56 - protein, 482 aa, Artificial Sequence
  Synthetic
  MISC_FEATURE: NicA2mut189; G333S, R351H, L424Q
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKESQLSKGAKLYVHVKQNLGHVFAFADEQQPLNWVQTHDYSDELGTILSITIARKETIDVNDRDAVTREVQKMFPGVEVLGTAAYDWTADPFSQGAWAAYGVGQLSRLKDLQAAEGRILFAGAETSNGWHANIDGAVESGLRAGREVKQLLS

SEQ ID NO: 57 - protein, 482 aa, Artificial Sequence
  Synthetic
  MISC_FEATURE: NicA2mut194; F174V, G260S, W427L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVVPRPHEPMFTERARELDKSSVLDRIKTLGLSRLQQAQINSYMALYAGETTDKFGLPGVLKLFACGGWNYDAFMDTETHYRIQGGTISLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGALAAYGVGQLSRLKDLQAAEGRILFAGAETSNGWHANIDGAVESGLRAGREVKQLLS

SEQ ID NO: 58 - protein, 482 aa, Artificial Sequence
  Synthetic
  MISC_FEATURE: NicA2mut202; A45V, D130V, I388F
MSDKTKTNEGFSRRSFIGSAAVVTAGVAGLGAIDAASATQKTNRVSTVKGGFDYDVVVVGGGFAGATAARECGLQGYRTLLLEARSRLGGRTFTSRFAGQEIEFGGAWVHWLQPHVWAEMQRYGLGVVEVPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETFDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 59 - protein, 482 aa, Artificial Sequence
  Synthetic
  MISC_FEATURE: NicA2mut22
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYDVGQLSRLKDLQAAEGRILFAGAETSNGWHVNIDGAVESGLRAGREVKQLLS

SEQ ID NO: 60 - protein, 482 aa, Artificial Sequence
  Synthetic
  MISC_FEATURE: NicA2mut40
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLINMDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRASREVKQLLS

SEQ ID NO: 61 - protein, 482 aa, Artificial Sequence
  Synthetic
  MISC_FEATURE: NicA2mut123; Q75H, P131S, P152S, I159V
MSDKTKTNEGFSRRSFIGSAAVVTAGVAGLGAIDAASATQKTNRASTVKGGFDYDVVVVGGGFAGATAARECGLHGYRTLLLEARSRLGGRTFTSRFAGQEIEFGGAWVHWLQPHVWAEMQRYGLGVVEDSLTNLDKTLIMYNDGSVESISSDEFGKNVRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 62 - protein, 482 aa, Artificial Sequence
  Synthetic
  MISC_FEATURE: NicA2mut118; K137R, L217P, N457T
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDRTLIMYNDGSVESISPDEFGKNIRIAFEKLCHDAWEVFPRPHEPMFTERARELDKSSVLDRIKTLGLSRLQQAQINSYMAPYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSTGWHANIDGAVESGLRAGREVKQLLS

SEQ ID NO: 63 - protein, 482 aa, Artificial Sequence
  Synthetic
  MISC_FEATURE: NicA2mut9
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEVPLTNLDKTLIMYNDGSVESISPDEFGKNIRVAFEKLCHDAWEVFPRPHEPMFTERARDLDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 64 - protein, 482 aa, Artificial Sequence
  Synthetic
  MISC_FEATURE: NicA2mut101
MSDKTKTNEGFSRRSFIGSAAVVTAGVAGLGAIDAASATQKTNRTSTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVKTHDYSDELGTILSVTIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 65 - protein, 482 aa, Artificial Sequence
  Synthetic
  MISC_FEATURE: NicA2mut105
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVQNPLTNLDKTLIMYNDGSVESIIPDEFGKNIHIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 66 - protein, 482 aa, Artificial Sequence
  Synthetic
  MISC_FEATURE: NicA2mut173; D130N, V303A, N311S, A395V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVENPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGAVVMTVPLSTYKHIGFTPALSKGKQRFIKEGQLSKGAKLYVHVKQNLGRVFAFADEQQPLNWVQTHDYSDELGTILSITIARKETIDVNDRDVVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 67 - protein, 482 aa, Artificial Sequence
  Synthetic
  MISC_FEATURE: NicA2mut6
MSDKTKTNEGFSRRSFIGSAAVVTAGVAGLGAIDAASATQKTNRASTVKGGFDYDVVVVGGSFAGATAARECGLHGYGTLLLEARSRLGGRSFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 68 - protein, 482 aa, Artificial Sequence
  Synthetic
  MISC_FEATURE: NicA2mut17
MSDKTKTNEGFSRRSFIGSAAVVTAGVASLGAIDAASATQKTNRASTVKGGFDYDVVVVGGGFAGATAARECGLQGYRTLLLEARSRLGGRTFTSRFAGQEIEIGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETNDKFGLPGVLKLFACGGWNYDAFMDTETHYRIQGGTIGLINAILTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 69 - protein, 482 aa, Artificial Sequence
  Synthetic
  MISC_FEATURE: NicA2mut119; S276N, K292N, K324E, R351H
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMNVPVTAVEQVNGGVKINTDDDEIITAGVVVMTVPLNTYKHIGFTPALSEGKQRFIKEGQLSKGAKLYVHVKQNLGHVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 70 - protein, 482 aa, Artificial Sequence
  Synthetic
  MISC_FEATURE: NicA2mut130; P131S, V277I, V282I
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDSLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSIPVTAIEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 71 - protein, 482 aa, Artificial Sequence
  Synthetic
  MISC_FEATURE: NicA2mut158; W108R, D130Y, T183I, M403I
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGARVHWLQPHVWAEMQRYGLGVVEYPLTNLDKTLIMYNDGSVESISPDEFGKNIRIAFEKLCHDAWEVFPRPHEPMFIERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKIFPGVEVLGTAAYDWTADPFSLGAWAAYGVGQLSRLKDLQAAEGRILFAGAETSNGWHANIDGAVESGLRAGREVKQLLS

SEQ ID NO: 72 - protein, 482 aa, Artificial Sequence
  Synthetic
  MISC_FEATURE: NicA2mut169; G50A, L217P, G260A, V345L
MSDKTKTNEGFSRRSFIGSAAVVTAGVAGLGAIDAASATQKTNRASTVKAGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMAPYAGETTDKFGLPGVLKLFACGGWNYDAFMDTETHYRIQGGTIALINAMLTDSGAEVRMSVPVTAVEQVNGGVKIKTDDDEIITAGVVVMTVPLNTYKHIGFTPALSKGKQRFIKEGQLSKGAKLYVHLKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 73 - protein, 482 aa, Artificial Sequence
  Synthetic
  MISC_FEATURE: NicA2mut183; W108R, D130Y, K337M, R398L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGARVHWLQPHVWAEMQRYGLGVVEYPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSMGAKLYVHVKQNLGRVFAFADEQQPLNWVQTHDYSDELGTILSITIARKETIDVNDRDAVTLEVQKMFPGVEVLGTAAYDWTADPFSLGAWAAYGVGQLSRLKDLQAAEGRILFAGAETSNGWHANIDGAVESGLRAGREVKQLLS

SEQ ID NO: 74 - protein, 482 aa, Artificial Sequence
  Synthetic
  MISC_FEATURE: NicA2mut198; E249G, H368R
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTGTHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 75 - protein, 482 aa, Artificial Sequence
  Synthetic
  MISC_FEATURE: NicA2mut23
MSDKTKTNEGFSRRSFIGSAAVVTAGVAGLGAIDAASATQKTNRASTVKGGFDYDVVVVGGGFAGATAARECGLQGYRTLLLEARSRLGGRTFASRFAGQEIEFGGAWVHWLQPHVWAEMQRYGLGVVEDSLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTTVEQVNGGVKIKTDDDEIITAAVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 76 - protein, 482 aa, Artificial Sequence
  Synthetic
  MISC_FEATURE: NicA2mut25
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEEPLTNLDKTLIMYNDGSVESISPDEFGKNIRIAFEKLCHDAWEVFPRPHEPMFTERARELDKSSVLDRIKTLGLSRLQQAQINSYMALYAGETTEKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGHVFAFADEQQPLNWVQTHDYSDELGTILSITIARKETIDVNDRDAVTREVQKMFPGVEVLGTAAYDWTADPFSLGAWAAYSVGQLSRLKDLQAAEGRILFAGAETSNGWHANIDGAVESGLRAGREVKQLLS

SEQ ID NO: 77 - protein, 484 aa, Artificial Sequence
  Synthetic
  MISC_FEATURE: NicA2mut10
MSDKTKTNEGFSRRSFIGSAAVVTAGVAGLGAIDAASATQKTNRASTVKDGFDYDVVVVGGGFAGATAARECGLQGYRTLLLEARSRLGGRIFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQPAELI

SEQ ID NO: 78 - protein, 482 aa, Artificial Sequence
  Synthetic
  MISC_FEATURE: NicA2mut117; P180L, 244V, T258A, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHELMFTERARELDKSSVLDRIKTLGLSRLQQAQINSYMALYAGETTDKFGLPGVLKLFACGGWNYDVFMDTETHYRIQGGAIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 79 - protein, 482 aa, Artificial Sequence
  Synthetic
  MISC_FEATURE: NicA2mut64
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWGVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKSAKLYVHVKQNLGRVFAFADEQQPLNWVQTHDYSDELGTILSITIARKETIDINDRDAVTREVQKMFPGVEVLGTAAYDWTADPFSLGAWAAYGVGQLSRLKDLQAAEGRILFAGAETSNGWLANIDGAVESGLRAGREVKQLLS

SEQ ID NO: 80 - protein, 482 aa, Artificial Sequence
  Synthetic
  MISC_FEATURE: NicA2mut174; T42A, R78H, D130V, K225N, F245Y
MSDKTKTNEGFSRRSFIGSAAVVTAGVAGLGAIDAASATQKANRASTVKGGFDYDVVVVGGGFAGATAARECGLQGYHTLLLEARSRLGGRTFTSRFAGQEIEFGGAWVHWLQPHVWAEMQRYGLGVVEVPLTNLDKTLIMYNDGSVESISPDEFGKNIRIAFEKLCHDAWEVFPRPHEPMFTERARELDKSSVLDRIKTLGLSRLQQAQINSYMALYAGETTDNFGLPGVLKLFACGGWNYDAYMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 81 - protein, 482 aa, Artificial Sequence
  Synthetic
  MISC_FEATURE: NicA2mut145; D130G, K157M, V173A, K191M
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGMNIRIAFEKLCHDAWEAFPRPHEPMFTERARELDMSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 82 - protein, 482 aa, Artificial Sequence
  Synthetic
  MISC_FEATURE: NicA2mut146; R85H, P229L, R351H, R437H
MSDKTKTNEGFSRRSFIGSAAVVTAGVAGLGAIDAASATQKTNRASTVKGGFDYDVVVVGGGFAGATAARECGLQGYRTLLLEAHSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLLGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGHVFAFADEQQPLNWVQTHDYSDELGTILSITIARKETIDVNDRDAVTREVQKMFPGVEVLGTAAYDWTADPFSLGAWAAYGVGQLSHLKDLQAAEGRILFAGAETSNGWHANIDGAVESGLRAGREVKQLLS

SEQ ID NO: 83 - protein, 482 aa, Artificial Sequence
  Synthetic
  MISC_FEATURE: NicA2mut152; T267I, W427L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLIDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGALAAYGVGQLSRLKDLQAAEGRILFAGAETSNGWHANIDGAVESGLRAGREVKQLLS

SEQ ID NO: 84 - protein, 482 aa, Artificial Sequence
  Synthetic
  MISC_FEATURE: NicA2mut147; G156C, T267S, W427L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFCKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLSDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGALAAYGVGQLSRLKDLQAAEGRILFAGAETSNGWHANIDGAVESGLRAGREVKQLLS

SEQ ID NO: 85 - protein, 482 aa, Artificial Sequence
  Synthetic
  MISC_FEATURE: NicA2mut167; D130G, D196H, M246T, K331N
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGKNIRIAFEKLCHDAWEVFPRPHEPMFTERARELDKSSVLHRIKTLGLSRLQQAQINSYMALYAGETTDKFGLPGVLKLFACGGWNYDAFTDTETHYRIQGGTIGLINAMLTDSGAEVRMSVPVTAVEQVNGGVKIKTDDDEIITAGVVVMTVPLNTYKHIGFTPALSKGKQRFINEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 86 - protein, 482 aa, Artificial Sequence
  Synthetic
  MISC_FEATURE: NicA2mut179; F104L, D130G, G270C, R393C
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSCAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDCDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 87 - protein, 482 aa, Artificial Sequence
  Synthetic
  MISC_FEATURE: NicA2mut192; G99D, D130V, E454D
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFADQEIEFGGAWVHWLQPHVWAEMQRYGLGVVEVPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGADTSNGWHANIDGAVESGLRAGREVKQLLS

SEQ ID NO: 88 - protein, 482 aa, Artificial Sequence
  Synthetic
  MISC_FEATURE: NicA2mut201; D130G, R187L, V231I, Y242N, E408D
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGKNIRIAFEKLCHDAWEVFPRPHEPMFTERALELDKSSVLDRIKTLGLSRLQQAQINSYMALYAGETTDKFGLPGILKLFACGGWNNDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVDVLGTAAYDWTADPFSLGAWAAYGVGQLSRLKDLQAAEGRILFAGAETSNGWHANIDGAVESGLRAGREVKQLLS

SEQ ID NO: 89 - protein, 482 aa, Artificial Sequence
  Synthetic
  MISC_FEATURE: NicA2mut35
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKILIMYNDGSVESISPDEFGKNIRIAFEKLCHDAWEVIPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDVDEIITAGVVVMTVPLNTYKHIGFTPALSKGKQRFIKEGQLSKGAKLYVHVKQNLGRVFAFADEQQPLNWVQTYDYSDELGTILSITIARKETIDVNDRDAVTREVQKMFPGVEVLGTAAYDWTADPFSLGAWAAYGVGQLSRLKDLQAAEGRILFAGAEASNGWHANIDGAVESGLRAGREVKQLLS

SEQ ID NO: 90 - protein, 482 aa, Artificial Sequence
  Synthetic
  MISC_FEATURE: NicA2mut7
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEVPLTNLDKTLIMYNDGSLESISPDEFSKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLSDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLDTAAYDWTADPFSLGAWAAYGVGQLSRLKDLQAAEGRILFAGAETSNGWHANIDGAVESGLRAGREVKQLLS

SEQ ID NO: 91 - protein, 482 aa, Artificial Sequence
  Synthetic
  MISC_FEATURE: NicA2mut65
MSDKTKTNEGFSRRSFIGSAAVVTAGVAGLGAIDAASATQKTNRARTVKGGFDYDVVVVGGGFAGATAARECGLQGYRTLLLEARSRLGGRTFTSRFAGQEIEFGGPWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSIPVTAVEQVNGGVKIKTDDDEFITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGRVVKQLLS

SEQ ID NO: 92 - protein, 482 aa, Artificial Sequence
  Synthetic
  MISC_FEATURE: NicA2mut137; V48A, F104L, D130Y,T267I, L435Q
MSDKTKTNEGFSRRSFIGSAAVVTAGVAGLGAIDAASATQKTNRASTAKGGFDYDVVVVGGGFAGATAARECGLQGYRTLLLEARSRLGGRTFTSRFAGQEIELGGAWVHWLQPHVWAEMQRYGLGVVEYPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLIDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQQSRLKDLQAAEGRILFAGAETSNGWHANIDGAVESGLRAGREVKQLLS

SEQ ID NO: 93 - protein, 482 aa, Artificial Sequence
  Synthetic
  MISC_FEATURE: NicA2mut144; D130G, P131Q, S192N, M306V,A473V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGQLTNLDKTLIMYNDGSVESISPDEFGKNIRIAFEKLCHDAWEVFPRPHEPMFTERARELDKNSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVVTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRVGREVKQLLS

SEQ ID NO: 94 - protein, 482 aa, Artificial Sequence
  Synthetic
  MISC_FEATURE: NicA2mut160; T39S, K157R, I161F,M246T, H368L, E373K
MSDKTKTNEGFSRRSFIGSAAVVTAGVAGLGAIDAASASQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGRNIRFAFEKLCHDAWEVFPRPHEPMFTERARELDKSSVLDRIKTLGLSRLQQAQINSYMALYAGETTDKFGLPGVLKLFACGGWNYDAFTDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTLDYSDKLGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 95 - protein, 482 aa, Artificial Sequence
  Synthetic
  MISC_FEATURE: NicA2mut180; G156D, H168Q, K292R, K324E, R351H
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFDKNIRIAFEKLCQDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIRTDDDEIITAGVVVMTVPLNTYKHIGFTPALSEGKQRFIKEGQLSKGAKLYVHVKQNLGHVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 96 - protein, 484 aa, Artificial Sequence
  Synthetic
  MISC_FEATURE: NicA2mut95
MSDKTKTNEGFSRRSFIGSAAVVTAGVAGLGAIDAASATQKTNRASTVKGGFDYDVVVVGGGVAGATAARECGLQGYRTLLLEARSRLGGRTFTSHFAGQEIEFGGAWVHWLQPHVWAEMQRYGLGVVENPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSIPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILNITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQPAELI

SEQ ID NO: 97 - protein, 482 aa, Artificial Sequence; identical to SEQ ID NO: 27
  Synthetic
  MISC_FEATURE: NicA2mut191; F104I, D130G, G156D, K199R, D295N, T381S

SEQ ID NO: 98 - protein, 482 aa, Artificial Sequence
  Synthetic
  MISC_FEATURE: NicA2mut2E3; A107T, D130G, T267I, L335V,N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLIDSGAEVRMSVPVTAVEQVNGGVKIKTDDDEIITAGVVVMTVPLNTYKHIGFTPALSKGKQRFIKEGQVSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 99 - protein, 482 aa, Artificial Sequence
  Synthetic
  MISC_FEATURE: NicA2mut2D5; K49N, F93L, F104L, V127M, D130S, L132R, F174C, T319I, E454D
MSDKTKTNEGFSRRSFIGSAAVVTAGVAGLGAIDAASATQKTNRASTVNGGFDYDVVVVGGGFAGATAARECGLQGYRTLLLEARSRLGGRTLTSRFAGQEIELGGAWVHWLQPHVWAEMQRYGLGMVESPRTNLDKTLIMYNDGSVESISPDEFGKNIRIAFEKLCHDAWEVCPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFIPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGADTSNGWHANIDGAVESGLRAGREVKQLLS

SEQ ID NO: 100 - protein, 482 aa, Artificial Sequence
  Synthetic
  MISC_FEATURE: NicA2mut2B5; A107T, P114Q, G317D, H368R,D389E, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQQHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIEVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 101 - protein, 482 aa, Artificial Sequence
  Synthetic
  MISC_FEATURE: NicA2mut2E4; F104I, G317D, H368R, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEIGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 102 - protein, 482 aa, Artificial Sequence
  Synthetic
  MISC_FEATURE: NicA2mut2F2; Q100H, A107T, L112Q, H115Y, H368R
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGHEIEFGGTWVHWQQPYVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 103 - protein, 482 aa, Artificial Sequence
  Synthetic
  MISC_FEATURE: NicA2mut2H3; S12I, A107T, D130G, T267I, N462S
MSDKTKTNEGFIRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLIDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 104 - protein, 482 aa, Artificial Sequence
  Synthetic
  MISC_FEATURE: NicA2mut2D9; T92A, F174V, E249G, A281T, I298V, F355L, H368R, E454D
MSDKTKTNEGFSRRSFIGSAAVVTAGVAGLGAIDAASATQKTNRASTVKGGFDYDVVVVGGGFAGATAARECGLQGYRTLLLEARSRLGGRAFTSRFAGQEIEFGGAWVHWLQPHVWAEMQRYGLGVVEDPLTNLDKTLIMYNDGSVESISPDEFGKNIRIAFEKLCHDAWEVVPRPHEPMFTERARELDKSSVLDRIKTLGLSRLQQAQINSYMALYAGETTDKFGLPGVLKLFACGGWNYDAFMDTGTHYRIQGGTIGLINAMLTDSGAEVRMSVPVTTVEQVNGGVKIKTDDDEVITAGVVVMTVPLNTYKHIGFTPALSKGKQRFIKEGQLSKGAKLYVHVKQNLGRVFALADEQQPLNWVQTRDYSDELGTILSITIARKETIDVNDRDAVTREVQKMFPGVEVLGTAAYDWTADPFSLGAWAAYGVGQLSRLKDLQAAEGRILFAGADTSNGWHANIDGAVESGLRAGREVKQLLS

SEQ ID NO: 105 - protein, 482 aa, Artificial Sequence
  Synthetic
  MISC_FEATURE: NicA2mut249; A69V, A107T, D130Y, K199R, P278Q, D295N, T381S
MSDKTKTNEGFSRRSFIGSAAVVTAGVAGLGAIDAASATQKTNRASTVKGGFDYDVVVVGGGFAGATAVRECGLQGYRTLLLEARSRLGGRTFTSRFAGQEIEFGGTWVHWLQPHVWAEMQRYGLGVVEYPLTNLDKTLIMYNDGSVESISPDEFGKNIRIAFEKLCHDAWEVFPRPHEPMFTERARELDKSSVLDRIRTLGLSRLQQAQINSYMALYAGETTDKFGLPGVLKLFACGGWNYDAFMDTETHYRIQGGTIGLINAMLTDSGAEVRMSVQVTAVEQVNGGVKIKTDNDEIITAGVVVMTVPLNTYKHIGFTPALSKGKQRFIKEGQLSKGAKLYVHVKQNLGRVFAFADEQQPLNWVQTHDYSDELGTILSISIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 106 - protein, 482 aa, Artificial Sequence
  Synthetic
  MISC_FEATURE: NicA2mut245; F104L, D130G, V231I, L335V, L349M, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGILKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQVSKGAKLYVHVKQNMGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 107 - protein, 482 aa, Artificial Sequence
  Synthetic
  MISC_FEATURE: NicA2mut244; G50S, F104L, A107T, D130G, G156D, S192C, F235L, G239A, A426T, L435Q
MSDKTKTNEGFSRRSFIGSAAVVTAGVAGLGAIDAASATQKTNRASTVKSGFDYDVVVVGGGFAGATAARECGLQGYRTLLLEARSRLGGRTFTSRFAGQEIELGGTWVHWLQPHVWAEMQRYGLGVVEGPLTNLDKTLIMYNDGSVESISPDEFDKNIRIAFEKLCHDAWEVFPRPHEPMFTERARELDKCSVLDRIKTLGLSRLQQAQINSYMALYAGETTDKFGLPGVLKLLACGAWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGTWAAYGVGQQSRLKDLQAAEGRILFAGAETSNGWHANIDGAVESGLRAGREVKQLLS

SEQ ID NO: 108 - protein, 482 aa, Artificial Sequence
  Synthetic
  MISC_FEATURE: NicA2mut240; F104L, D130G, E184G, M246T, P278S, I291V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEGPLTNLDKTLIMYNDGSVESISPDEFGKNIRIAFEKLCHDAWEVFPRPHEPMFTGRARELDKSSVLDRIKTLGLSRLQQAQINSYMALYAGETTDKFGLPGVLKLFACGGWNYDAFTDTETHYRIQGGTIGLINAMLTDSGAEVRMSVSVTAVEQVNGGVKVKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 109 - protein, 482 aa, Artificial Sequence
  Synthetic
  MISC_FEATURE: NicA2mut239; F104I, D130A
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEIGGAWVHWLQPHVWAEMQRYGLGVVEAPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 110 - protein, 482 aa, Artificial Sequence
  Synthetic
  MISC_FEATURE: NicA2mut237; W108R, D130Y, T183I, G202D, S379N
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGARVHWLQPHVWAEMQRYGLGVVEYPLTNLDKTLIMYNDGSVESISPDEFGKNIRIAFEKLCHDAWEVFPRPHEPMFIERARELDKSSVLDRIKTLDLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILNITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 111 - protein, 482 aa, Artificial Sequence
  Synthetic
  MISC_FEATURE: NicA2mut234; F104L, G317D, D357E, H368R
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFAEEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 112 - protein, 482 aa, Artificial Sequence; identical to SEQ ID NO: 57
  Synthetic
  MISC_FEATURE: NicA2mut233; F174V, G260S, W427L

SEQ ID NO: 113 - protein, 482 aa, Artificial Sequence
  Synthetic
  MISC_FEATURE: NicA2mut232; F174V, G260S, H368P, W427L
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVVPRPHEPMFTERARELDKSSVLDRIKTLGLSRLQQAQINSYMALYAGETTDKFGLPGVLKLFACGGWNYDAFMDTETHYRIQGGTISLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTPDYSDELGTILSITIARKETIDVNDRDAVTREVQKMFPGVEVLGTAAYDWTADPFSLGALAAYGVGQLSRLKDLQAAEGRILFAGAETSNGWHANIDGAVESGLRAGREVKQLLS

SEQ ID NO: 114 - protein, 482 aa, Artificial Sequence
  Synthetic
  MISC_FEATURE: NicA2mut229; F104I, D130G, G156D, K199R, A209T, F355L, H368Y
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEIGGAWVHWLQPHVWAEMQRYGLGVVEGPLTNLDKTLIMYNDGSVESISPDEFDKNIRIAFEKLCHDAWEVFPRPHEPMFTERARELDKSSVLDRIRTLGLSRLQQTQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFALADEQQPLNWVQTYDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 115 - protein, 482 aa, Artificial Sequence
  Synthetic
  MISC_FEATURE: NicA2mut228; K314Q, H368R, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYQHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 116 - protein, 482 aa, Artificial Sequence
  Synthetic
  MISC_FEATURE: NicA2mut223; W171C, D243N, Q366R, S379N, D394N
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDACEVFPRPHEPMFTERARELDKSSVLDRIKTLGLSRLQQAQINSYMALYAGETTDKFGLPGVLKLFACGGWNYNAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVRTHDYSDELGTILNITIARKETIDVNDRNAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 117 - protein, 482 aa, Artificial Sequence
  Synthetic
  MISC_FEATURE: NicA2mut220; V48A, F104L, D130Y, M246T, H368L, E373K, L441M
MSDKTKTNEGFSRRSFIGSAAVVTAGVAGLGAIDAASATQKTNRASTAKGGFDYDVVVVGGGFAGATAARECGLQGYRTLLLEARSRLGGRTFTSRFAGQEIELGGAWVHWLQPHVWAEMQRYGLGVVEYPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFTDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTLDYSDKLGTILSITIARKETIDVNDRDAVTREVQKMFPGVEVLGTAAYDWTADPFSLGAWAAYGVGQLSRLKDMQAAEGRILFAGAETSNGWHANIDGAVESGLRAGREVKQLLS

SEQ ID NO: 118 - protein, 482 aa, Artificial Sequence
  Synthetic
  MISC_FEATURE: NicA2mut219; K157R, I161F, M246T, H368R, L449V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGRNIRFAFEKLCHDAWEVFPRPHEPMFTERARELDKSSVLDRIKTLGLSRLQQAQINSYMALYAGETTDKFGLPGVLKLFACGGWNYDAFTDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHANIDGAVESGLRAGREVKQLLS

SEQ ID NO: 119 - protein, 482 aa, Artificial Sequence
  Synthetic
  MISC_FEATURE: NicA2mut253; F104L, G317D, H368R, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLLS

SEQ ID NO: 120 - protein, 484 aa, Artificial Sequence
  Synthetic
  MISC_FEATURE: NicA2mut260; F104L, G317D, H368R, L449V, N462S, L481P, S482E, 483L, 484I
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLPELI

SEQ ID NO: 121 - protein, 482 aa, Artificial Sequence
  Synthetic
  MISC_FEATURE: NicA2mut254; A107T, D130G, T267I, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLIDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 122 - protein, 482 aa, Artificial Sequence; identical to SEQ ID NO: 119
  Synthetic
  MISC_FEATURE: NicA2mut255; F104L, G317D, H368R, L449V, N462S

SEQ ID NO: 123 - protein, 482 aa, Artificial Sequence
  Synthetic
  MISC_FEATURE: NicA2mut302; A107T, L112M, D357E, H368R, G406D, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWMQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFAEEQQPLNWVQTRDYSDELGTILSITIARKETIDVNDRDAVTREVQKMFPDVEVLGTAAYDWTADPFSLGAWAAYGVGQLSRLKDLQAAEGRIVFAGAETSNGWHASIDGAVESGLRAGREVKQLLS

SEQ ID NO: 124 - protein, 482 aa, Artificial Sequence
  Synthetic
  MISC_FEATURE: NicA2mut303; Y54F, V59I, F63L, A107T, V127M, D130S, L132R, F235I, T280K, G317D, E408D, P421L
MSDKTKTNEGFSRRSFIGSAAVVTAGVAGLGAIDAASATQKTNRASTVKGGFDFDVVVIGGGLAGATAARECGLQGYRTLLLEARSRLGGRTFTSRFAGQEIEFGGTWVHWLQPHVWAEMQRYGLGMVESPRTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLIACGGWNYDAFMDTETHYRIQGGTIGLINAMLTDSGAEVRMSVPVKAVEQVNGGVKIKTDDDEIITAGVVVMTVPLNTYKHIDFTPALSKGKQRFIKEGQLSKGAKLYVHVKQNLGRVFAFADEQQPLNWVQTHDYSDELGTILSITIARKETIDVNDRDAVTREVQKMFPGVDVLGTAAYDWTADLFSLGAWAAYGVGQLSRLKDLQAAEGRILFAGAETSNGWHANIDGAVESGLRAGREVKQLLS

SEQ ID NO: 125 - protein, 482 aa, Artificial Sequence
  Synthetic
  MISC_FEATURE: NicA2mut304; V48A, F52L, F104L, W171R, E249G, H368R, T381S, N462S
MSDKTKTNEGFSRRSFIGSAAVVTAGVAGLGAIDAASATQKTNRASTAKGGLDYDVVVVGGGFAGATAARECGLQGYRTLLLEARSRLGGRTFTSRFAGQEIELGGAWVHWLQPHVWAEMQRYGLGVVEDPLTNLDKTLIMYNDGSVESISPDEFGKNIRIAFEKLCHDAREVFPRPHEPMFTERARELDKSSVLDRIKTLGLSRLQQAQINSYMALYAGETTDKFGLPGVLKLFACGGWNYDAFMDTGTHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSISIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 126 - protein, 482 aa, Artificial Sequence
  Synthetic
  MISC_FEATURE: NicA2mut305; F93L, F104L, V127M, D130S, L132R, S213T, T222S, A281T, L374M, M403I
MSDKTKTNEGFSRRSFIGSAAVVTAGVAGLGAIDAASATQKTNRASTVKGGFDYDVVVVGGGFAGATAARECGLQGYRTLLLEARSRLGGRTLTSRFAGQEIELGGAWVHWLQPHVWAEMQRYGLGMVESPRTNLDKTLIMYNDGSVESISPDEFGKNIRIAFEKLCHDAWEVFPRPHEPMFTERARELDKSSVLDRIKTLGLSRLQQAQINTYMALYAGESTDKFGLPGVLKLFACGGWNYDAFMDTETHYRIQGGTIGLINAMLTDSGAEVRMSVPVTTVEQVNGGVKIKTDDDEIITAGVVVMTVPLNTYKHIGFTPALSKGKQRFIKEGQLSKGAKLYVHVKQNLGRVFAFADEQQPLNWVQTHDYSDEMGTILSITIARKETIDVNDRDAVTREVQKIFPGVEVLGTAAYDWTADPFSLGAWAAYGVGQLSRLKDLQAAEGRILFAGAETSNGWHANIDGAVESGLRAGREVKQLLS

SEQ ID NO: 127 - protein, 482 aa, Artificial Sequence
  Synthetic
  MISC_FEATURE: NicA2mut306; F93L, F104L, V127M, D130S, L132R, I198F, K331N
MSDKTKTNEGFSRRSFIGSAAVVTAGVAGLGAIDAASATQKTNRASTVKGGFDYDVVVVGGGFAGATAARECGLQGYRTLLLEARSRLGGRTLTSRFAGQEIELGGAWVHWLQPHVWAEMQRYGLGMVESPRTNLDKTLIMYNDGSVESISPDEFGKNIRIAFEKLCHDAWEVFPRPHEPMFTERARELDKSSVLDRFKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFINEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 128 - protein, 482 aa, Artificial Sequence
  Synthetic
  MISC_FEATURE: NicA2mut307; F104L, A107T, S146I, G317D, H368R, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGTWVHWLQPHVWAEMQRYGLGVVEDPLTNLDKTLIMYNDGIVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLLS

SEQ ID NO: 129 - protein, 482 aa, Artificial Sequence
  Synthetic
  MISC_FEATURE: NicA2mut313; F104L, E129V, S192N, E249G, G317D, H368R, T381S, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVVDPLTNLDKTLIMYNDGSVESISPDEFGKNIRIAFEKLCHDAWEVFPRPHEPMFTERARELDKNSVLDRIKTLGLSRLQQAQINSYMALYAGETTDKFGLPGVLKLFACGGWNYDAFMDTGTHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSISIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 130 - DNA, 1449 bp, Artificial Sequence; identical to SEQ ID NO: 12
  Synthetic
  misc_feature: Wild-type nicA2

SEQ ID NO: 131 - protein, 481 aa, Artificial Sequence
  Synthetic
  MISC_FEATURE: Wild type NicA2
SDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRILFAGAETSNGWHANIDGAVESGLRAGREVKQLLS

SEQ ID NO: 132 - protein, 482 aa, Artificial Sequence
  Synthetic
  MISC_FEATURE: NicA2mut314; G51S, G73S, R96S, F104L, A107T, L112M, E249G, S276C, H368R, G411S, N462S
MSDKTKTNEGFSRRSFIGSAAVVTAGVAGLGAIDAASATQKTNRASTVKGSFDYDVVVVGGGFAGATAARECSLQGYRTLLLEARSRLGGRTFTSSFAGQEIELGGTWVHWMQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTGTHYRIQGGTIGLINAMLTDSGAEVRMCVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLSTAAYDWTADPFSLGAWAAYGVGQLSRLKDLQAAEGRILFAGAETSNGWHASIDGAVESGLRAGREVKQLLS

SEQ ID NO: 133 - protein, 482 aa, Artificial Sequence
  Synthetic
  MISC_FEATURE: NicA2mut315; A107T, L112M, F235Y, G260S, A339V, Q359R, H368R, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGTWVHWMQPHVWAEMQRYGLGVVEDPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLYACGGWNYDAFMDTETHYRIQGGTISLINAMLTDSGAEVRMSVPVTAVEQVNGGVKIKTDDDEIITAGVVVMTVPLNTYKHIGFTPALSKGKQRFIKEGQLSKGVKLYVHVKQNLGRVFAFADERQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 134 - protein, 484 aa, Artificial Sequence
  Synthetic
  MISC_FEATURE: NicA2mut320; F104L, A107T, D130S, T319I, L449V, N462S, L480P, L481A, S482E
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGTWVHWLQPHVWAEMQRYGLGVVESPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFIPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQPAELI

SEQ ID NO: 135 - protein, 482 aa, Artificial Sequence
  Synthetic
  MISC_FEATURE: NicA2mut321; F104L, A107T, S146I, K191N, Q210H, G317D, H368R, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGTWVHWLQPHVWAEMQRYGLGVVEDPLTNLDKTLIMYNDGIVESISPDEFGKNIRIAFEKLCHDAWEVFPRPHEPMFTERARELDNSSVLDRIKTLGLSRLQQAHINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIDFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLLS

SEQ ID NO: 136 - protein, 482 aa, Artificial Sequence
  Synthetic
  MISC_FEATURE: NicA2mut323; F104L, A107T, D130S, M215L, E221D, T319I, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGTWVHWLQPHVWAEMQRYGLGVVESPLTNLDKTLIMYNDGSVESISPDEFGKNIRIAFEKLCHDAWEVFPRPHEPMFTERARELDKSSVLDRIKTLGLSRLQQAQINSYLALYAGDTTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFIPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLLS

SEQ ID NO: 137 - protein, 482 aa, Artificial Sequence
  Synthetic
  MISC_FEATURE: NicA2mut324; F104L, D130N, H368R, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVENPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 138 - protein, 482 aa, Artificial Sequence
  Synthetic
  MISC_FEATURE: nicA2mut325; D130G, D296E, G317S, Q359E, S371R, S379N, T418S, L449V
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIEFGGAWVHWLQPHVWAEMQRYGLGVVEGPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDEEIITAGVVVMTVPLNTYKHISFTPALSKGKQRFIKEGQLSKGAKLYVHVKQNLGRVFAFADEEQPLNWVQTHDYRDELGTILNITIARKETIDVNDRDAVTREVQKMFPGVEVLGTAAYDWSADPFSLGAWAAYGVGQLSRLKDLQAAEGRIVFAGAETSNGWHANIDGAVESGLRAGREVKQLLS

SEQ ID NO: 139 - protein, 482 aa, Artificial Sequence
  Synthetic
  MISC_FEATURE: NicA2mut326; F104L, D130S, H368R, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGAWVHWLQPHVWAEMQRYGLGVVESPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKTDDDEIITAGVVVMTVPLNTYKHIGFTPALSKGKQRFIKEGQLSKGAKLYVHVKQNLGRVFAFADEQQPLNWVQTRDYSDELGTILSITIARKETIDVNDRDAVTREVQKMFPGVEVLGTAAYDWTADPFSLGAWAAYGVGQLSRLKDLQAAEGRILFAGAETSNGWHASIDGAVESGLRAGREVKQLLS

SEQ ID NO: 140 - protein, 482 aa, Artificial Sequence
  Synthetic
  MISC_FEATURE: NicA2mut329; F104L, A107T, D130S, T293S, T307P, T319I, L449V, N462S
MSDKTKTNEGFSRRSFIGSAAVVTAGVAGLGAIDAASATQKTNRASTVKGGFDYDVVVVGGGFAGATAARECGLQGYRTLLLEARSRLGGRTFTSRFAGQEIELGGTWVHWLQPHVWAEMQRYGLGVVESPLTNLDKTLIMYNDGSVESISPDEFGKNIRIAFEKLCHDAWEVFPRPHEPMFTERARELDKSSVLDRIKTLGLSRLQQAQINSYMALYAGETTDKFGLPGVLKLFACGGWNYDAFMDTETHYRIQGGTIGLINAMLTDSGAEVRMSVPVTAVEQVNGGVKIKSDDDEIITAGVVVMPVPLNTYKHIGFIPALSKGKQRFIKEGQLSKGAKLYVHVKQNLGRVFAFADEQQPLNWVQTHDYSDELGTILSITIARKETIDVNDRDAVTREVQKMFPGVEVLGTAAYDWTADPFSLGAWAAYGVGQLSRLKDLQAAEGRIVFAGAETSNGWHASIDGAVESGLRAGREVKQLLS